(19) United States
(12) Patent Application Publication
(10) Pub. No.: US 2023/0138428 A1
(43) Pub. Date: May 4, 2023

(54) CHIMERIC RECEPTORS FOR USE IN ENGINEERED CELLS

(71) Applicant: QUELL THERAPEUTICS LIMITED, London, Greater London (GB)

(72) Inventors: Marc MARTINEZ-LLORDELLA, London, Greater London (GB); Elisa PETRIS, London, Greater London (GB); Frederick DEAR, London, Greater London (GB); Maria ALONSO-FERRERO, London, Greater London (GB)

(21) Appl. No.: 17/802,018

(22) PCT Filed: Feb. 24, 2021
(86) PCT No.: PCT/EP2021/054578

§ 371 Date: Aug. 24, 2022

(30) Foreign Application Priority Data
Feb. 25, 2020 (GB) 2002620.9
Feb. 25, 2020 (GB) 2002622.5

(51) Int. Cl. C07K 14/725; C07K 14/705

(52) U.S. Cl. CPC C07K 14/7051; C07K 14/70521; A61K 2039/5156

(57) ABSTRACT

The present invention provides a nucleic acid molecule comprising a polynucleotide sequence encoding a chimeric receptor, particularly a chimeric antigen receptor (CAR), wherein said chimeric receptor comprises an endodomain comprising (i) a STAT5 association motif, and a JAK1 and/or JAK2 binding motif and (ii) a JAK3 binding motif, wherein (i) and (ii) are connected by a linker or hinge.

### FIELD OF THE INVENTION

The present invention relates to engineered cells, particularly engineered T cells (for example, regulatory T cells) and therapeutic uses of such cells. In particular, the invention relates to engineered cells, particularly engineered T cells (for example, regulatory T cells) that are less susceptible to microenvironments with limited IL-2 availability and which have a stable cell (e.g., Treg) phenotype. The cells are engineered to express chimeric receptors and provided herein are nucleic acids encoding chimeric receptors, particularly chimeric antigen receptors (CARs).

### BACKGROUND TO THE INVENTION

Regulatory T cells (Tregs) are immune cells with suppressive function that control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases and in transplantation. Treg immunotherapies usually involve isolation, culture and expansion of Tregs followed by infusion into patients. As part of this process, Tregs may be incubated with cytokines, drugs, other cells or antigens in order to improve their viability and function and/or to confer them enhanced reactivity against specific antigens. These same objectives can be achieved by genetically engineering Tregs to target a predetermined antigen, for example via a chimeric antigen receptor (CAR).

The growth factor interleukin-2 (IL-2) is essential for the homeostasis of Tregs (generation, proliferation, survival), as well as for their suppressive function and phenotypic stability. Activated conventional T cells (Tcons) are the main source of IL-2 in vivo. Tregs, in contrast, cannot produce IL-2 and depend on paracrine access to IL-2 produced by Tcons present in the microenvironment.

The availability of IL-2 has a critical impact on the therapeutic effects of Tregs expanded in vitro and transferred into patients. This is due to the following: 1) in vitro expansion protocols typically require high concentrations of IL-2, which renders Tregs highly dependent on this cytokine; 2) the concentration of IL-2 is often reduced in patients as a result of the administration of immunosuppressive drugs; and 3) within the inflamed tissue microenvironment access to IL-2 is often limited. Liver transplantation constitutes a particularly challenging indication, given that the levels of IL-2 in the inflamed liver are known to be reduced, which is further aggravated by the routine use of calcineurin inhibitors, which substantially decrease the capacity of Tcons to produce IL-2. The administration of low doses exogenous IL-2 restores the Treg dysfunction induced by calcineurin inhibitors and promotes the accumulation of Tregs in the liver. However, a concern with the therapeutic use of low-dose Treg is the risk of simultaneously activating Tcons, which can enhance tissue damage.

WO 2017/218850 describes engineering Tregs which constitutively express STAT5 in order to provide a productive IL-2 signal. However, several challenges can be predicted with this approach. Constitutive STAT5 expression provides a risk that the engineered Tregs may exert non-specific powerful immunosuppression and, due to their high proliferative rate, they may overgrow the endogenous Treg pool and reduce their TCR repertoire, which could result in autoimmunity. Finally, these engineered Tregs may pose risk of transformation, considering that mutations on STAT5 are known to promote T-cell prolymphocytic leukaemia, and that STAT5 is constitutively activated in many cancers.

Regulatory T cells express FOXP3 and conventional T cells can be differentiated towards a regulatory phenotype ex vivo by expressing FOXP3 in those cells. Loss of FOXP3 expression is associated with a loss of suppressive function in regulatory T cells and a potential return to an effector phenotype.

Accordingly, there remains a need for approaches to produce engineered Tregs which are less susceptible to microenvironments with limited IL-2 availability and approaches to improve the effectiveness of engineered Tregs to proliferate, survive and function in subjects who have been administered immunosuppressive drugs.

### SUMMARY OF THE INVENTION

The present inventors have developed an engineered regulatory T cell (Treg) which is capable of providing a productive IL-2 signal upon binding of the Treg to a predetermined antigen and which has a stable regulatory phenotype. Thus, the engineered Tregs of the present invention address the problem associated with the high IL-2 dependence of adoptively transferred Tregs without requiring exogenous IL-2 to be administered and by providing a productive IL-2 signal in an antigen-specific manner, whilst maintaining phenotype and function. Particularly, the productive IL-2 signal may be provided through the inclusion of particular motifs within the endodomain of a chimeric receptor expressed within a cell (namely a STAT5 association motif, a JAK1 and/or JAK2 binding motif, and a JAK3 binding motif). The inventors have particularly identified that the use of a linker between specific motifs within the endodomain of the chimeric receptor (e.g., a CAR), can provide an enhanced IL-2 signal which results in increased survival of the engineered cells. Particularly, there may be a linker between a sub-domain or sequence in the endodomain comprising the STAT5 and JAK1 and/or JAK2 motifs and a sub-domain or sequence in the endodomain comprising the JAK3 binding motif.

The present invention further particularly provides a Treg cell with a stable phenotype and an IL2 signalling capacity through the co-expression of exogenous FOXP3 and a CAR having an endodomain comprising a STAT5 association motif, a JAK1 and/or JAK2 binding motif and a JAK3 binding motif.

Thus, the present invention provides a nucleic acid molecule comprising a polynucleotide sequence encoding a chimeric receptor, particularly a chimeric antigen receptor (CAR), wherein said chimeric receptor (particularly said CAR) comprises an endodomain comprising (i) a STAT5 association motif, and a JAK1 and/or JAK2 binding motif and (ii) a JAK3 binding motif, wherein (i) and (ii) are connected by a linker or hinge.

In a further aspect, the nucleic acid molecule may additionally encode FOXP3 and thus the invention provides a nucleic acid comprising (a) a first polynucleotide sequence encoding FOXP3, and (b) a second polynucleotide sequence encoding a chimeric receptor (particularly a chimeric antigen receptor (CAR)) wherein said chimeric receptor comprises an endodomain comprising (i) a STAT5 association motif, and a JAK1 and/or JAK2 binding motif and (ii) a JAK3 binding motif, wherein (i) and (ii) are connected by a linker or hinge.

Although said first polynucleotide sequence may be positioned 5′ or 3′ relative to the second polynucleotide within the nucleic acid molecule, particularly, the first polynucleotide sequence is positioned 5′ (i.e. upstream) relative to said second polynucleotide sequence. It will be appreciated by a skilled person that a co-expression sequence may be conveniently positioned between said first and said second polynucleotide sequence within said nucleic acid molecule.

In a second aspect, the present invention provides a vector comprising a nucleic acid molecule of the invention.

In a third aspect, the present invention provides an engineered cell (particularly a T cell, e.g., a Treg, or an NK cell) comprising a nucleic acid molecule of the invention or a vector of the invention.

In a fourth aspect, the present invention provides a pharmaceutical composition comprising an engineered cell (e.g., Treg cell) of the invention.

In a fifth aspect, the present invention provides the engineered cell (e.g., Treg cell) of the invention or the pharmaceutical composition of the invention for use in therapy.

In another aspect the present invention provides an engineered cell (e.g., Treg cell) for use in induction of tolerance to a transplant; treating and/or preventing graft-versus-host disease (GvHD), an autoimmune or allergic disease; to promote tissue repair and/or tissue regeneration; or to ameliorate, or treat, inflammation (e.g., chronic inflammation secondary to metabolic disorders); wherein said engineered cell (e.g., Treg cell) comprises a nucleic acid molecule of the invention or a vector of the invention.

In another aspect the present invention provides a pharmaceutical composition comprising an engineered cell (e.g., Treg) of the invention for use in induction of tolerance to a transplant; treating and/or preventing GvHD, an autoimmune or allergic disease; to promote tissue repair and/or tissue regeneration; or to ameliorate or treat inflammation (e.g., chronic inflammation secondary to metabolic disorders).

The invention further relates to a method of inducing tolerance to a transplant; treating and/or preventing GvHD, an autoimmune or allergic disease; or to promote tissue repair and/or tissue regeneration; or to ameliorate or treat inflammation (e.g., chronic inflammation secondary to metabolic disorders) which comprises the step of administering an engineered cell (e.g., a Treg) or a pharmaceutical composition according to the present invention to a subject.

The present invention also provides the use of an engineered cell (e.g., Treg) according to the present invention in the manufacture of a medicament for inducing tolerance to a transplant; treating and/or preventing cellular and/or humoral transplant rejection; treating and/or preventing GvHD, an autoimmune or allergic disease; or to promote tissue repair and/or tissue regeneration; or to ameliorate or treat inflammation (e.g., chronic inflammation secondary to metabolic disorders).

More generally in these various aspects the engineered cell may be used in the treatment of or prevention of cancer, or an infectious, neurodegenerative, inflammatory, autoimmune or allergic disease or any condition associated with an unwanted or deleterious immune response. In particular, where the cell is a Treg or other immunosuppressive cell, the cell may be used for inducing immunosuppression (i.e. for suppressing an unwanted or deleterious immune response), for example to improve and/or prevent immune-mediated organ damage in inflammatory disorders, autoimmune or allergic diseases or conditions, and in transplantation.

Particularly, a cell of the invention may be used to treat amyotrophic lateral sclerosis (ALS).

Suitably, the subject (particularly a human subject) may be a transplant recipient and the invention is directed to induction of tolerance to a transplant (e.g. a transplanted organ). In particular, the subject may be a transplant recipient undergoing immunosuppression therapy.

The invention further provides a method for producing an engineered cell (e.g., Treg cell) of the invention comprising introducing a nucleic acid molecule of the invention or a vector of the invention into a cell (e.g., a pluripotent cell, particularly an iPSC, a Tcon cell or into a Treg cell), and a cell obtainable from the method.

More generally, the invention provides a method for producing an engineered cell (e.g. a Treg cell) comprising introducing into a cell (e.g., Treg cell) (a) a first polynucleotide sequence encoding FOXP3 and (b) a second polynucleotide sequence encoding a chimeric receptor (e.g., a chimeric antigen receptor (CAR)) wherein said chimeric receptor (e.g., CAR) comprises an endodomain comprising (i) a STAT5 association motif, and a JAK1 and/or JAK2 binding motif and (ii) a JAK3 binding motif, wherein (i) and (ii) are connected by a linker or hinge and wherein said first and second polynucleotide sequences are comprised in the same or different nucleic acid molecules.

The cell may also be a pluripotent cell, such as an iPSC, or a Tcon. The introduction of a nucleic acid encoding FOXP3 may allow differentiation or conversion to a Treg cell. Additional agents may further be encoded by a nucleic acid of the invention, or encoded on a separate nucleic acid to further aid any necessary step of differentiation (particularly, additional transcription factors). Alternatively or additionally, other agents may be added to the culture to aid with differentiation, for example TGFβ.

In particular, the Treg may be defined by expression of CD25 and immunosuppressive activity.

The first and second polynucleotide sequences may be in the same or different vectors.

The invention further provides a method for increasing the suppressive activity and/or persistence of a Treg cell comprising introducing a nucleic acid molecule of the invention or a vector of the invention into a Tcon cell or into a Treg cell or comprising introducing one or more (e.g. a) nucleic acid molecule(s) comprising (i) a first polynucleotide sequence encoding FOXP3 and (ii) a second polynucleotide sequence encoding a chimeric receptor (e.g., a chimeric antigen receptor (CAR)) wherein said chimeric receptor (e.g., CAR) comprises an endodomain comprising (i) a STAT5 association motif, and a JAK1 and/or JAK2 binding motif and (ii) a JAK3 binding motif wherein (i) and (ii) are connected by a linker or hinge, into a Tcon or a Treg cell.

The present invention thus provides an engineered cell (e.g., Treg) comprising a nucleic acid molecule or vector encoding a chimeric receptor (e.g., CAR), which chimeric receptor (e.g., CAR) provides a STAT5-mediated pro-survival signal to the cell (e.g., Treg) exclusively upon chimeric receptor binding to its cognate ligand (e.g., CAR binding to its cognate antigen), and optionally, encoding FOXP3 which imparts a stable T regulatory phenotype upon the engineered cell (particularly Treg). In particular, after ligand (e.g., antigen) recognition, the present chimeric receptors (e.g., CARs) may cluster and a signal is transmitted to the engineered cell (e.g., Treg) via the intracellular signaling domain (endodomain) of the chimeric receptor (e.g., CAR). Because the present chimeric receptor (e.g., CAR) comprises an endodomain which comprises a STAT5 association motif, a JAK1 and/or a JAK2 binding motif, and a JAK3 binding motif, clustering of the present chimeric receptor (e.g., CAR) leads to STAT5, JAK1 and/or JAK2 and JAK3 recruitment and activation; and thus provides a signal that enhances the function and the survival of the engineered cell (e.g., Treg) in a ligand-specific (e.g., an antigen-specific) manner without being dependent on the availability of IL-2 in the microenvironment. The optional expression of exogenous FOXP3 allows Treg cells to maintain their regulatory function regardless of the status of endogenous FOXP3 expression.

The engineered cells (e.g., Tregs) of the present invention may be particularly effective in providing a survival advantage to the engineered cells (e.g., CAR-Tregs) after ligand/antigen recognition compared to the general T cell population of the subject. In particular, in the context of, e.g., transplantation where the use of immunosuppressive drugs reduces the availability of IL-2, the STAT5 signalling of the present chimeric receptor Tregs (e.g., CAR-Tregs) provides additional survival and functional effects on the cells of the invention in an otherwise disadvantageous microenvironment. The signalling provided for by the present invention is particularly enhanced by the inclusion of a JAK3 binding motif in combination with a STAT5 association motif and a JAK1 and/or JAK2 binding motif within the chimeric receptor (e.g. CAR), and with the inclusion of a linker or hinge between the domains.

In an additional aspect, the present invention provides a nucleic acid molecule comprising (i) a first polynucleotide sequence encoding FOXP3 and (ii) a second polynucleotide sequence encoding a chimeric receptor (e.g., a chimeric antigen receptor (CAR)) wherein said chimeric receptor (e.g. said CAR) comprises an endodomain comprising a STAT5 association motif, a JAK1 and/or JAK2 binding motif and a JAK3 binding motif, wherein said first and second polynucleotide sequences are operably linked to the same promoter.

Although said first polynucleotide sequence may be positioned 5′ or 3′ relative to the second polynucleotide within the nucleic acid molecule, particularly, the first polynucleotide sequence is positioned 5′ (i.e. upstream) relative to said second polynucleotide sequence. It will be appreciated by a skilled person that a co-expression sequence may be conveniently positioned between said first and said second polynucleotide sequence within said nucleic acid molecule. Vectors comprising the nucleic acid encoding the chimeric receptor (e.g., CAR), cells comprising the nucleic acid, vector, or chimeric receptor (e.g., CAR), and methods of making a cell, and therapeutic uses of the cell are further encompassed.

### BRIEF DESCRIPTION OF THE FIGURES

FIG. 1—Diagram illustrating CAR constructs of the invention

FIG. 2—Exemplary designs of anti-HLA.A2 IL2R CAR constructs

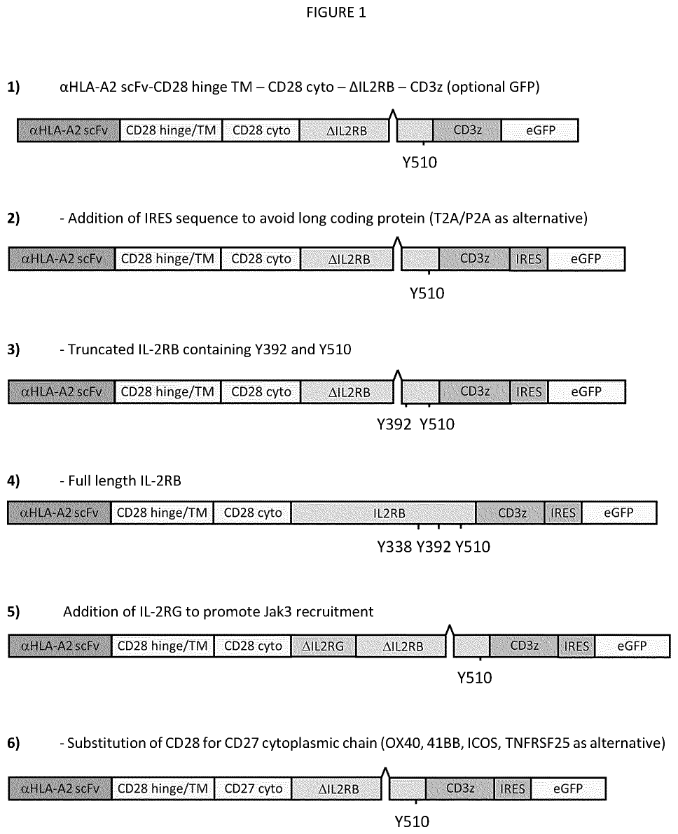

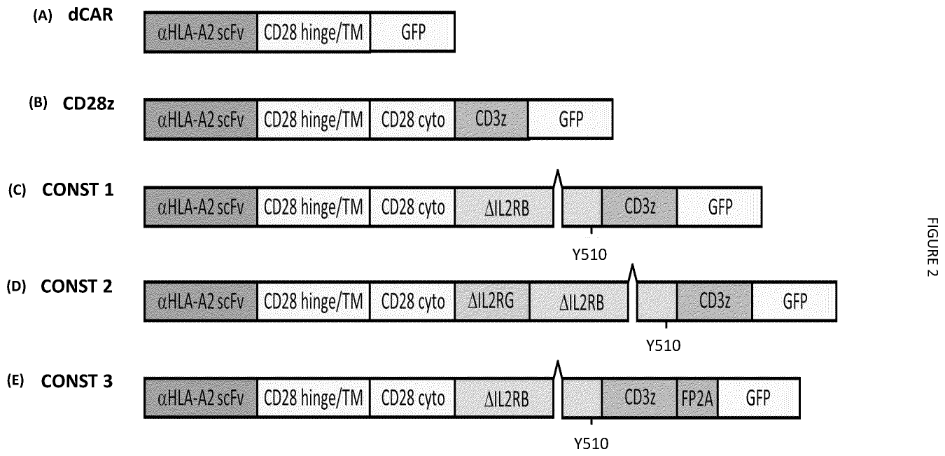

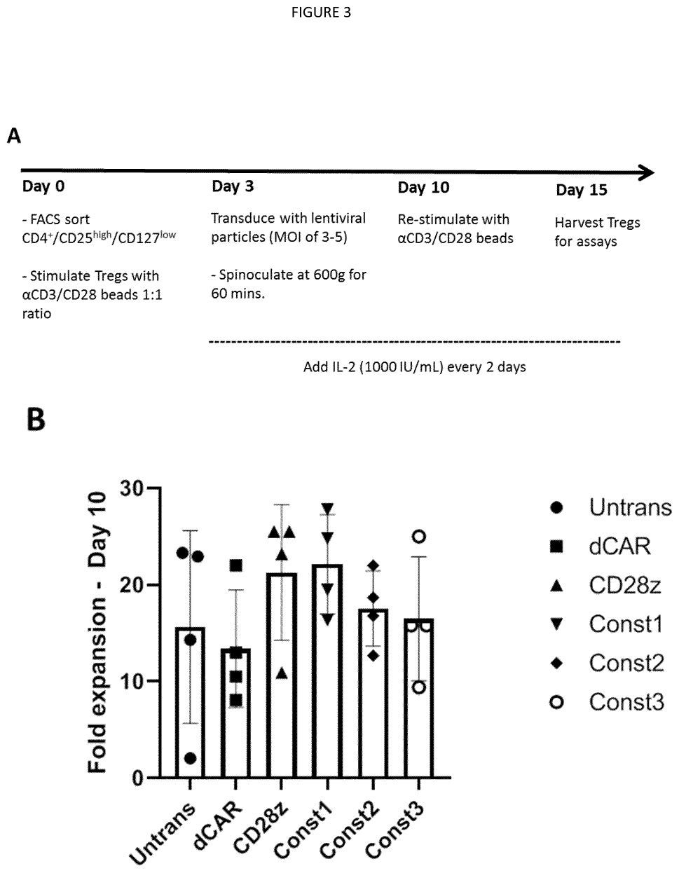

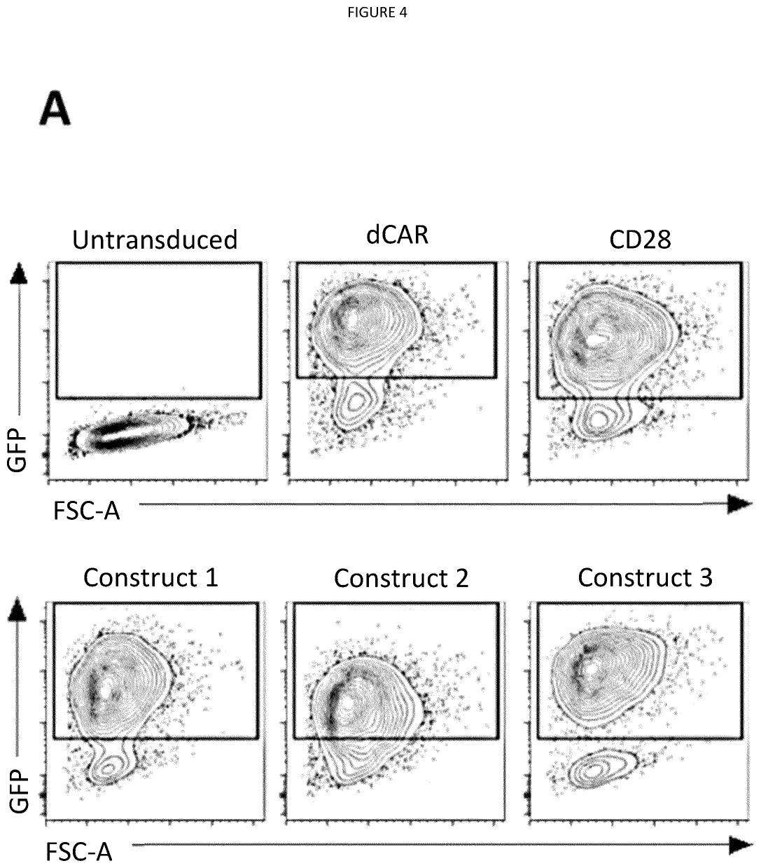

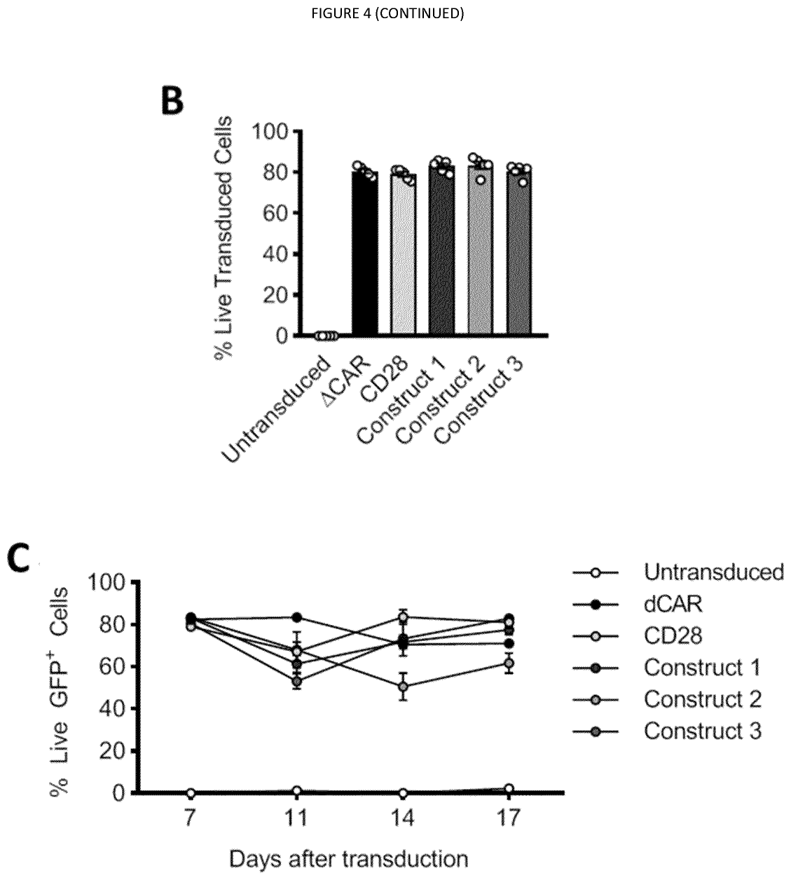

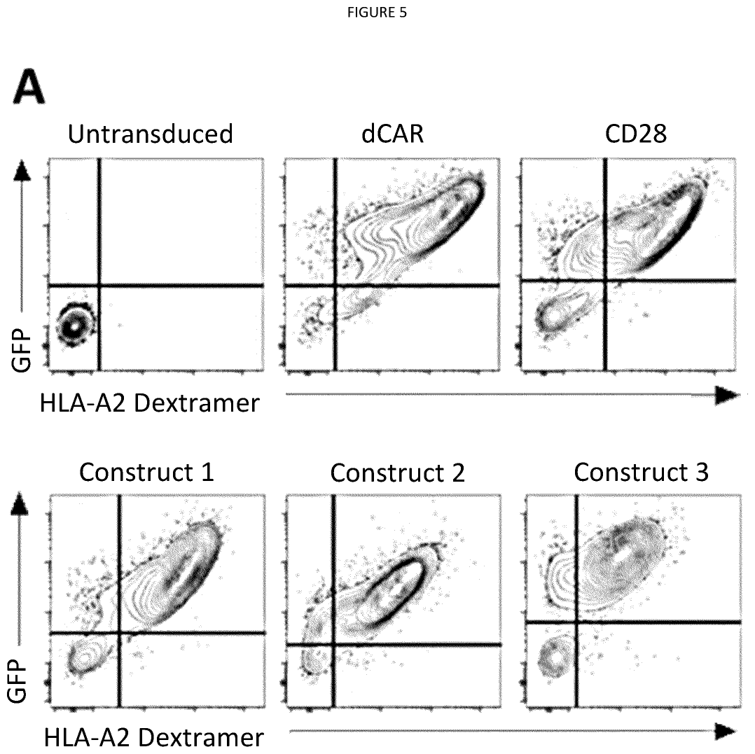

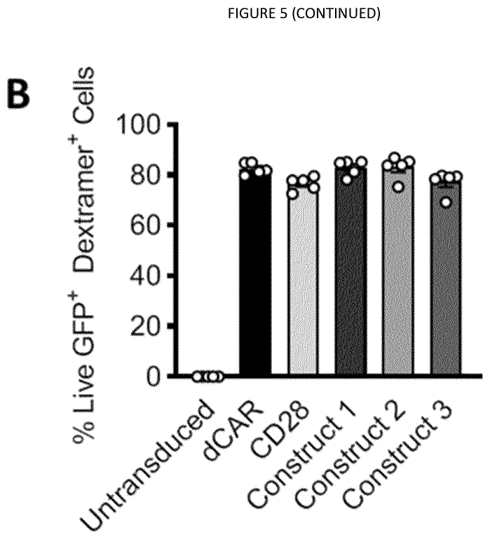

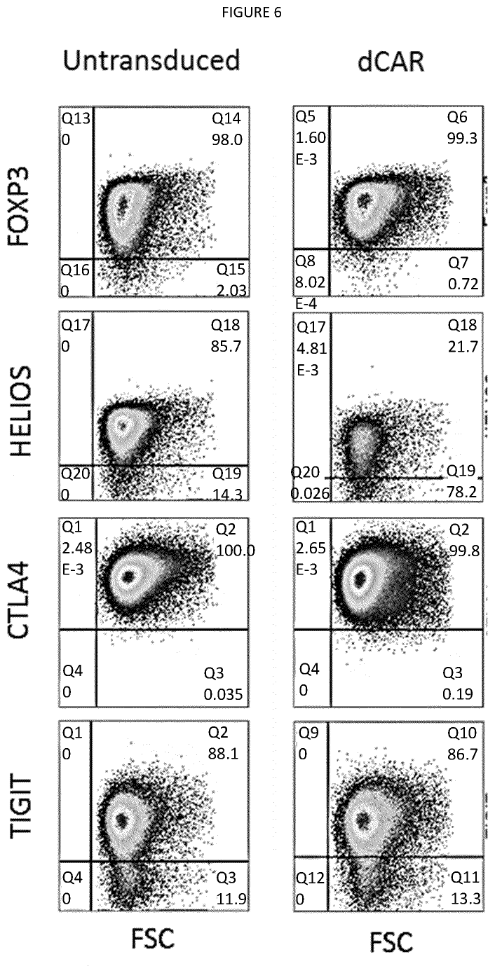

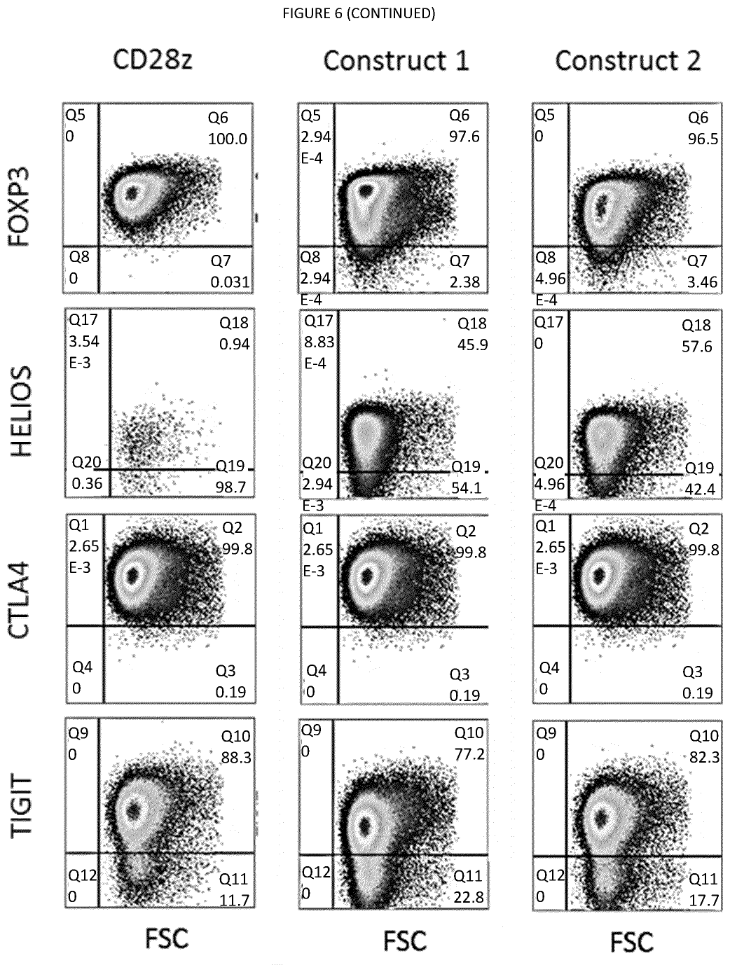

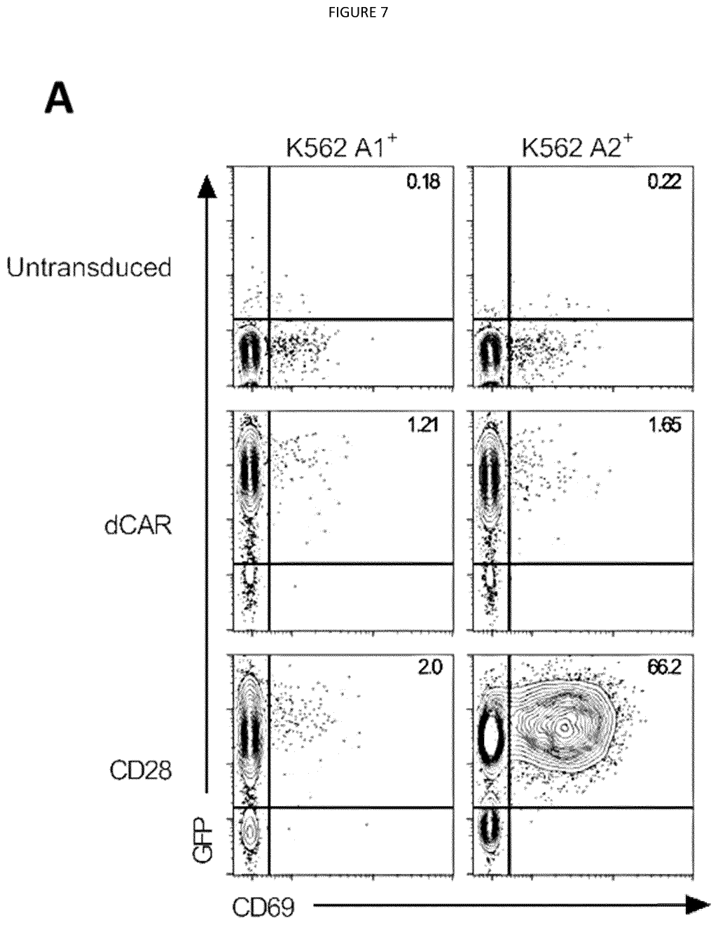

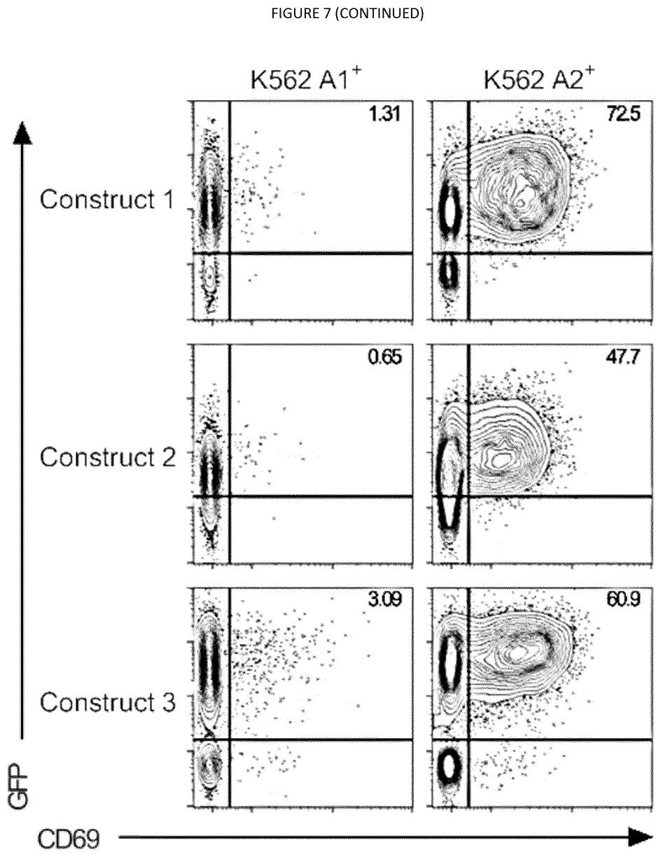

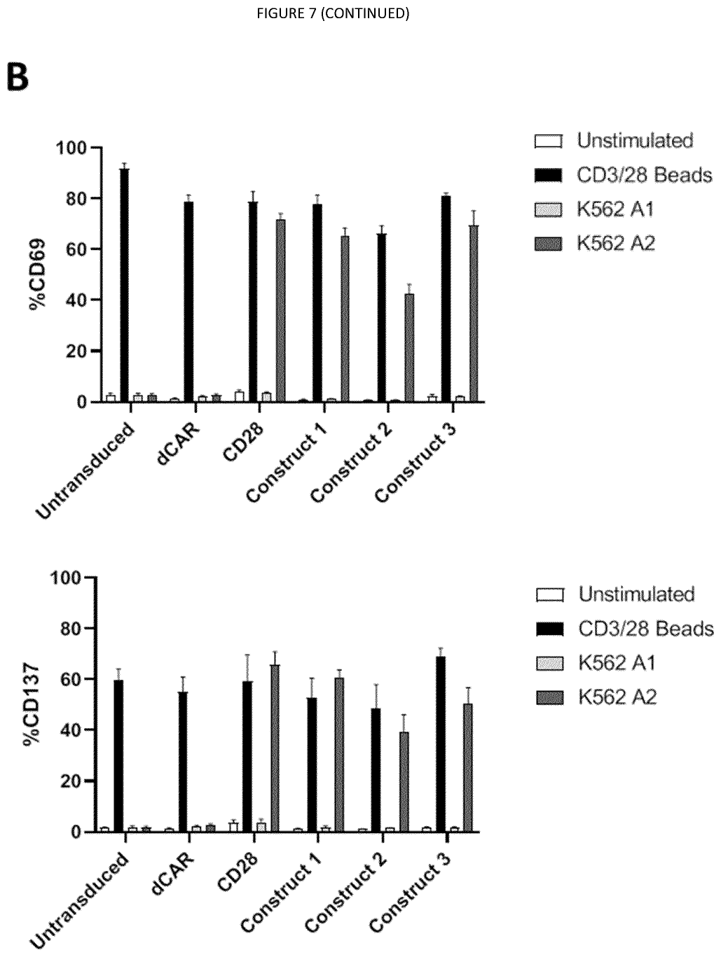

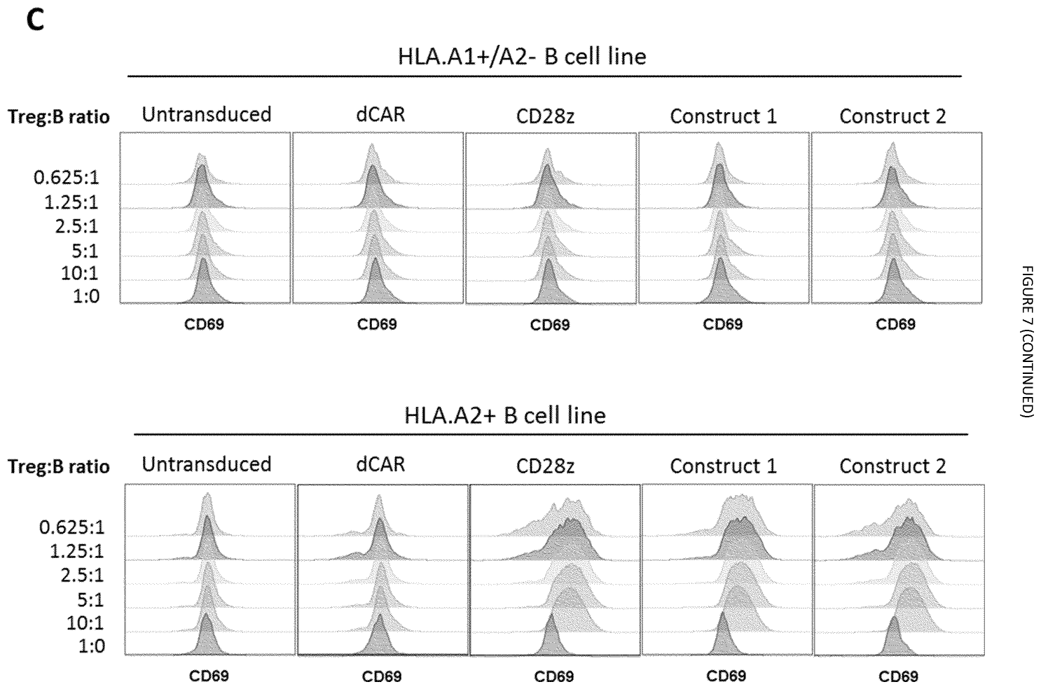

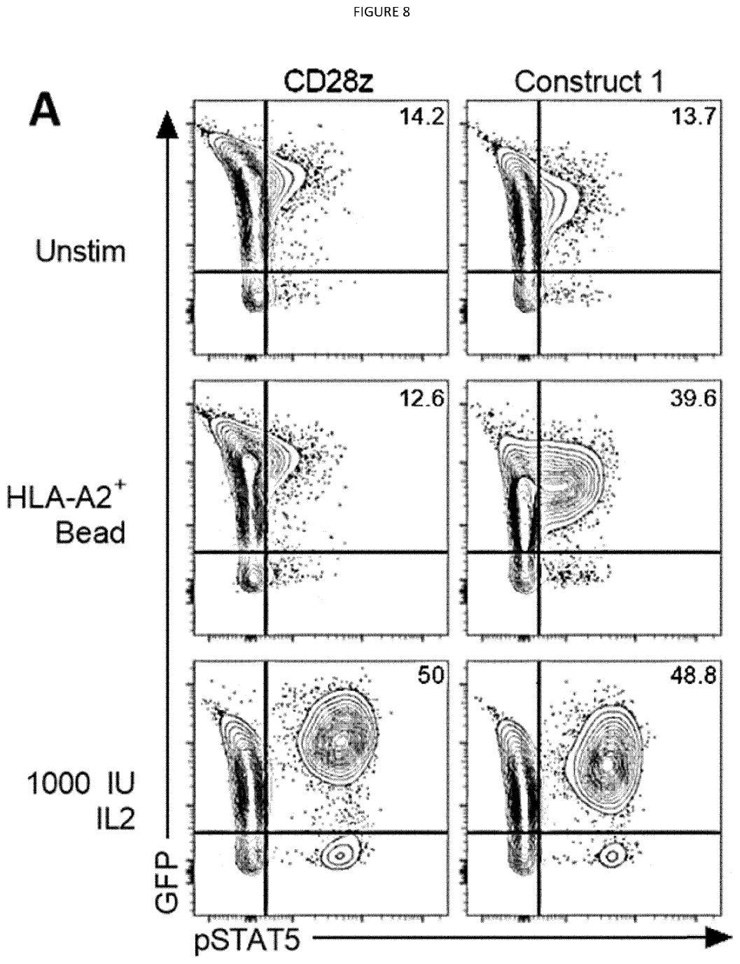

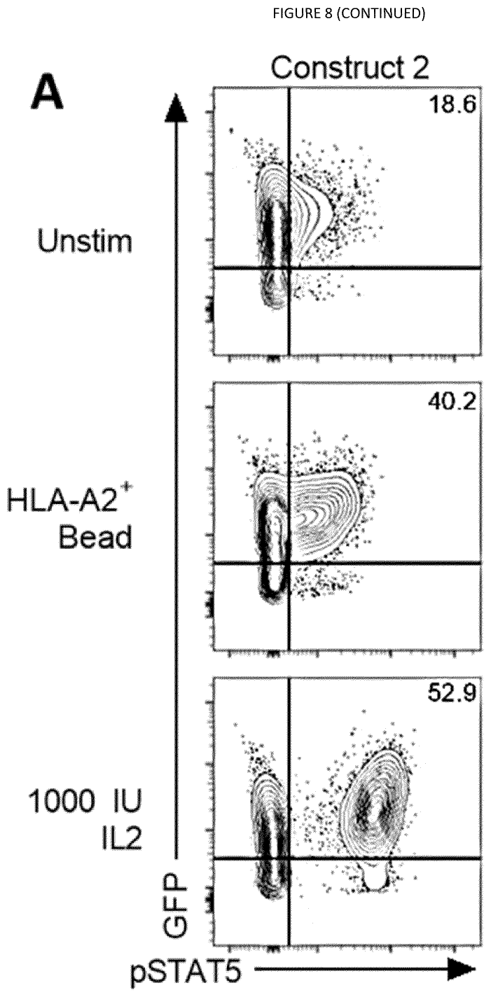

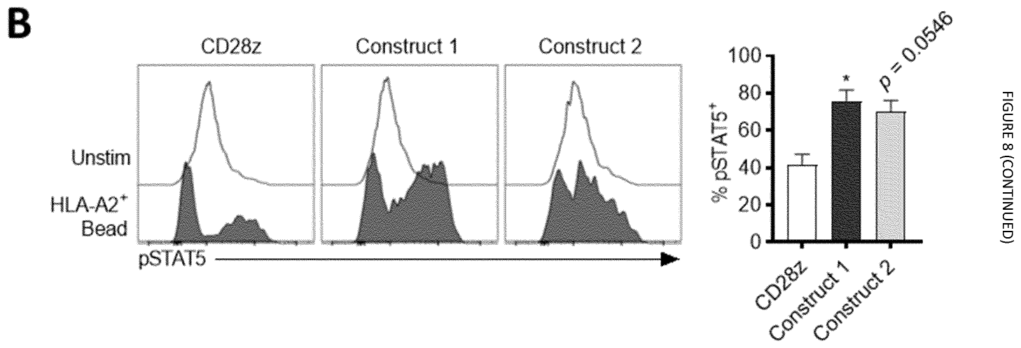

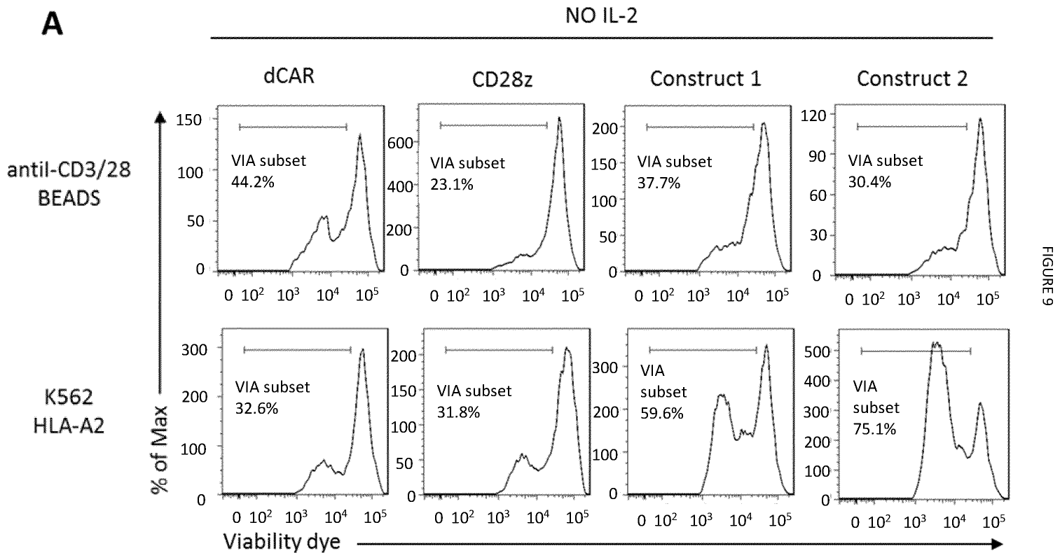

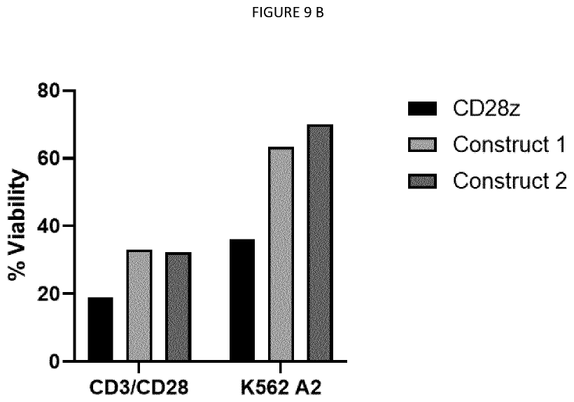

Schematics of exemplary anti-HLA.A2 CAR constructs including different combinations of IL2R endodomain. (A) dCAR construct: HLA.A2 scFv antigen recognition domain; CD28 hinge domain; CD28 TM and eGFP. (B) CD28z construct: HLA.A2 scFv antigen recognition domain; CD28 hinge domain; CD28 TM; CD28 signaling domain; CD3z signaling domain and eGFP. (C) IL2R Construct 1: HLA.A2 scFv antigen recognition domain; CD28 hinge domain; CD28 TM; CD28 signaling domain; truncated IL2RB endodomain; CD3z signaling domain and eGFP. (D) IL2R Construct 1: HLA.A2 scFv antigen recognition domain; CD28 hinge domain; CD28 TM; CD28 signaling domain; truncated IL2RG; truncated IL2RB endodomain; CD3z signaling domain and eGFP. (E) IL2R Construct 1: HLA.A2 scFv antigen recognition domain; CD28 hinge domain; CD28 TM; CD28 signaling domain; truncated IL2RB endodomain; CD3z signaling domain; FP2A cleavage domain and eGFP.

FIG. 3—Generation of anti-HLA.A2 IL2R CAR-Tregs

Schematic illustration showing the generation and expansion of anti-HLA.A2 IL2R CAR-Tregs. (A) Isolated CD4+CD25hiCD127low cells were isolated and activated with anti-CD3/CD28 beads. Three days after activation Tregs were transduced with lentivirus containing the HLA.A2-CAR and the GFP reported gene. Fresh media and 1000 IU/ml IL-2 were added every 2 days. Transduced and untransduced Tregs were cultured during 10 days and GFP was measured to assess transduction efficacy. Tregs were further expanded with fresh anti-CD3/CD28 beads. (B) Fold change expansion of Tregs untransduced or transduced with different CAR constructs on day 10 after activation.

FIG. 4—Quantification of transduction efficacy of anti-HLA.A2 IL2R constructs over time

GFP expression was analysed on Tregs untransduced and transduced with CAR constructs at different time points after cell activation. (A) Representative contour plots of GFP expression from HLA-A2 IL2R CAR Tregs 7 days following transduction. (B) Quantification of GFP⁺ CAR Tregs among live CD4+ cells 7 days following transduction. (C) Quantification of GFP expression from HLA-A2 IL2R CAR Tregs over time.

FIG. 5—Quantification of cell surface expression of anti-HLA.A2 IL2R CAR constructs on transduced Tregs

Membrane expression of CAR construct on untransduced and transduced Tregs was analysed by PE-conjugated HLA-A*0201/CINGVCWTV dextramers (Immudex, Copenhagen, Denmark). (A) Representative contour plots of GFP+ Dextramer+ CAR Tregs 7 days following transduction. (B) Quantification of Dextramer+ cells among the GFP+ Tregs on day 7 after transduction.

FIG. 6—Phenotypic characterization of CAR Tregs after polyclonal cell expansion

Tregs were cultured and expanded for 15 days in the presence of anti-CD3/CD28 activation beads and IL-2. Treg related markers FOXP3, HELIOS, CTLA4 and TIGIT were analysed by FACS on untransduced and transduced Tregs to assess phenotypic lineage stability on day 15 of culture.

FIG. 7—Evaluation of the antigen-specificity of anti-HLA.A2 IL2R CAR Tregs

Untransduced and transduced Tregs were cultured for 18 hours in the presence of different stimulus. CD69 and CD137 activation markers were analysed to assess specific and unspecific cell activation. (A) Representative contour plots showing the expression CD69 in response to culture with K562 cells transduced with HLA.A1 or HLA.A2 molecules. GFP signal was used to select the transduced Tregs. (B) Quantification of CD69 and CD137 expression on Tregs 18 hours after culture with media alone (unstimulated), anti-CD3/CD28 beads (unspecific stimulation), K562-HLA.A1 and K562-HLA.A2 cells. (C) Representative histograms showing CD69 expression on Tregs after 18 hours culture with HLA.A1 and HLA.A2 B cell lines. Different cell to cell ratios were used.

FIG. 8—STAT5 phosphorylation analysis as an indicator of IL2R CAR signaling

Transduced CAR Tregs were rested overnight in culture media without IL2. STAT5 phosphorylation of Tregs was assessed by FACS analysis 10 and 120 minutes after culture with media alone, 1000 IU/ml IL-2 or in the presence of HLA.A2-Ig based artificial APCs (produced following the protocol described at DOI: 10.3791/2801). (A) Contour plots showing the expression of GFP and phosphoSTAT5 on transduced CAR-Tregs after 10 minutes culture with media alone, HLA.A2 beads at 1:1 ratio and 1000 IU/ml IL-2. (B) Histograms showing the phosphorylation of STAT5 of Tregs cultured for 120 minutes with HLA.A2 beads 1:1 ratio or media alone (unstim).

FIG. 9—Evaluation of Treg survival after unspecific and HLA.A2 specific activation in the absence of IL-2

CAR transduced Tregs with different constructs were cultured with anti-CD3/28 activation beads and K562.A2 expression cells without the presence of IL-2. Cell survival was assessed 7 days after activation by FACS analysis. (A) Representatives histograms of CAR-Tregs showing cell survival of GFP+ cells based on Viability dye staining on day 7 after activation without IL-2. (B) Percentage of viable cells on GFP+ Tregs after 7 days of culture whit anti-CD3/28 beads and K562-HLA.A2 cells in absence of IL-2.

FIG. 10—Treg suppression potency test: Evaluate the immunoregulatory function of Tregs by analysing the modulation of co-stimulatory molecules on B cells

B cell expression of CD80 and CD86 after co-culture with Tregs was analysed to evaluate the capacity of Tregs to reduce the expression of co-stimulatory molecules on antigen presenting cells. Fixed number of alive A2-expressing B cells (20K/well) were co-cultured with titrated numbers of Treg products (A2-negative donors) (200, 100, 50, 25, 12.5K) overnight. FACS analysis of CD86 and CD80 co-stimulatory markers on B cells.

FIG. 11A—Illustrative HLA-A2-specific CAR construct designs

Schematic diagram of illustrative vectors encoding a HLA-A2-specific CAR (denoted as A2 CAR): Construct F-C: illustrates a construct encoding 5′-FOXP3-P2A-A2 CAR-3′; Construct R-C: illustrates a construct encoding 5′-R-P2A-A2 CAR-3′, where R is another gene; Construct C: illustrates a construct encoding the A2 CAR only; Construct C-R: illustrates a construct encoding 5′-A2 CAR-P2A-R-3′, where R is another gene.

FIG. 11B—Generation of FOXP3/HLA-A2 CAR-Tregs

Schematic illustration showing the generation and expansion of the FOXP3/HLA-A2 CAR-Tregs. Phoenix-GP (P.gp) cells, a retroviral packaging line stably expressing gag pol, were seeded at 1×10⁶ cells/10 mm cell culture dish. The next day CD4+CD25hiCD127low cells were isolated and activated with anti-CD3/CD28 beads in the absence of IL-2. On the same day P.gp cells were transfected with envelope and constructs encoding FOXP3/HLA.A2-CAR using Fugene transfection reagent. Two days after activation Tregs were transduced with g-retrovirus containing and supplemented with IL-2. Every 2 days cells were supplemented with additional media and IL-2. HLA.A2 dextramers were used to assess transduction efficacy at day 6. Tregs were further expanded with fresh anti-CD3/CD28 beads.

FIG. 12—HLA-A2-specific CAR and FOXP3 expression in transduced Tregs

FIG. 12 shows the HLA-A2-specific CAR (A2 CAR) expression levels, FOXP3 expression levels and the expression levels of another gene, R, in Tregs transduced with Construct F-C, Construct R-C, Construct C, and Construct C-R, compared to mock control Tregs, as determined by flow cytometry.

FIG. 12A shows CD3+CD4+ gated FACS plots for Dextramer (HLA) against SSC-A for the mock Tregs and for Tregs transduced with Construct F-C, Construct R-C, Construct C, and Construct C-R. Tregs transduced with each construct expressed the HLA-A2-specific CAR.

FIG. 12B shows CD3+CD4+ Dextramer+ gated FACS plots for expression of FOXP3 against expression of another gene, R, for the mock Tregs and for Tregs transduced with Construct F-C, Construct R-C, Construct C, and Construct C-R. The gene, R, was expressed in high levels in Tregs transduced with both Construct R-C and Construct C-R. FOXP3 was expressed in all Tregs, but was noticeably higher in Construct F-C, particularly when compared to expression of the HLA-A2-specific CAR alone (Construct C).

FIG. 13 —HLA-A2-specific CAR and FOXP3 expression in transduced and expanded Tregs

FIG. 13 shows the HLA-A2-specific CAR (A2 CAR) expression levels, FOXP3 expression levels and the expression levels of another gene, R, in expanded Tregs transduced with Construct F-C, Construct R-C, Construct C, and Construct C-R, compared to mock control Tregs, as determined by flow cytometry.

FIG. 13A shows CD3+CD4+ gated FACS plots for Dextramer (HLA) against SSC-A for the mock expanded Tregs and for Tregs transduced with Construct F-C, Construct R-C, Construct C, and Construct C-R and subsequently expanded. Tregs transduced with each construct still expressed the HLA-A2-specific CAR after expansion.

FIG. 13B shows CD3+CD4+ Dextramer+ gated FACS plots for expression of FOXP3 against expression of another gene, R, for the mock expanded Tregs and for Tregs transduced with Construct F-C, Construct R-C, Construct C, and Construct C-R and subsequently expanded. FOXP3 expression decreased in Tregs transduced with Construct R-C, Construct C, and Construct C-R after expansion. In contrast, FOXP3 expression did not decrease in Tregs transduced Construct F-C after expansion. Consequently, FOXP3 expression was substantially higher in Construct F-C after expansion, particularly when compared to expression of the HLA-A2-specific CAR alone (Construct C).

FIG. 14—Schematic of a CAR of the invention

FIG. 14 shows a schematic of a CAR of the invention, which comprises an endodomain comprising truncated IL2Rgamma and truncated IL2Rbeta domains, joined by a linker.

FIG. 15—Exemplary designs of the anti-HLA.A2 IL2R CAR constructs

FIG. 15 shows schematic diagrams of exemplary anti-HLA.A2 CAR constructs including different combinations of IL2R endodomain and flexible linker sequences. Control (SEQ ID NO: 182): HLA.A2 scFv antigen recognition domain; CD8 hinge domain (SEQ ID NO: 194); CD8 transmembrane (TM) (SEQ ID NO: 195); CD28 signaling domain; CD3ζ signaling domain. QTX01-CI (SEQ ID NO: 183): HLA.A2 scFv antigen recognition domain; CD8 hinge domain; CD8 TM; CD28 signaling domain; truncated IL2Rβ signaling domain; CD3ζ signaling domain. QTX01-CII (SEQ ID NO: 184): HLA.A2 scFv antigen recognition domain; CD8 hinge domain; CD8 TM; CD28 signaling domain; truncated IL2Rγ signaling domain; truncated IL2Rβ signaling domain; CD3ζ signaling domain. QTX01-CIII (SEQ ID NO: 185): HLA.A2 scFv antigen recognition domain; CD8 hinge domain; CD8 TM; CD28 signaling domain; truncated IL2Ry signaling domain; CD3ζ signaling domain. QTX01-CIV (SEQ ID NO: 186): HLA.A2 scFv antigen recognition domain; CD8 hinge domain; CD8 TM; CD28 signaling domain; truncated IL2Ry signaling domain; (GGGGS)₃ flexible linker (SEQ ID NO: 155); truncated IL2Rβ signaling domain; CD3ζ signaling domain. QTX01-CV (SEQ ID NO: 187): HLA.A2 scFv antigen recognition domain; CD8 hinge domain; CD8 TM; CD28 signaling domain; truncated IL2Ry signaling domain; (GGGGS)₄ flexible linker (SEQ ID NO: 158); truncated IL2Rβ signaling domain; CD3ζ signaling domain. QTX01-CVI (SEQ ID NO: 188): HLA.A2 scFv antigen recognition domain; CD8 hinge domain; CD8 TM; CD28 signaling domain; truncated IL2Ry signaling domain; (Gly)₆ flexible linker (SEQ ID NO: 159); truncated IL2Rβ signaling domain; CD3ζ signaling domain. QTX01-CVII (SEQ ID NO: 189): HLA.A2 scFv antigen recognition domain; CD8 hinge domain; CD8 TM; CD28 signaling domain; truncated IL2Ry signaling domain; (Gly)₈ flexible linker (SEQ ID NO: 160); truncated IL2Rβ signaling domain; CD3ζ signaling domain. QTX01-CVIII (SEQ ID NO: 190): HLA.A2 scFv antigen recognition domain; CD8 hinge domain; CD8 TM; CD28 signaling domain; truncated IL2Ry signaling domain; KESGSVSSEQLAQFRSLD flexible linker (SEQ ID NO: 166); truncated IL2Rβ signaling domain; CD3ζ signaling domain. QTX01-CIX (SEQ ID NO: 191): HLA.A2 scFv antigen recognition domain; CD8 hinge domain; CD8 TM; CD28 signaling domain; truncated IL2Ry signaling domain; EGKSSGSGSESKST flexible linker (SEQ ID NO: 167); truncated IL2Rβ signaling domain; CD3ζ signaling domain. QTX01-CX (SEQ ID NO: 192): HLA.A2 scFv antigen recognition domain; CD8 hinge domain; CD8 TM; CD28 signaling domain; truncated IL2Ry signaling domain; GSAGSAAGSGEF flexible linker (SEQ ID NO: 168); truncated IL2Rβ signaling domain; CD3ζ signaling domain.

FIG. 16—Cell surface expression of anti-HLA.A2 IL2R CAR and RQR8, and intracellular expression of FoxP3 in transduced Tregs.

FIG. 16 shows (A) Percentage of CD4⁺ CD25⁺ Tregs cells expressing membrane-located CAR constructs on transduced Tregs (B) CAR constructs Mean Fluorescence Intensity (MFI) Geometric Mean (Geo Mean) for each Treg condition. (C) Percentage of CD4⁺ CD25⁺ Tregs cells expressing membrane-located reporter molecule RQR8 (SEQ ID NO: 193) (D) QBEND/10 MFI values of RQR8 (SEQ ID NO: 193) for each Treg condition (E) Percentage of CD4⁺ CD25⁺ Treg cells expressing intracellular FoxP3 transcription factor on transduced Tregs. (F) MFI values of FoxP3 for each Treg condition is shown. Bars show mean values+ standard deviation (SD). N=2 to 16 individual donors.

FIG. 17—Evaluation of the Antigen-Specific Activation Capacity in Anti-HLA.A2 IL2R CAR Tregs

FIG. 17 shows the levels of T cell activation markers (A) CD69 and (B) CD137 as determined by flow cytometry after culture of transduced CAR Tregs with HLA-A1⁺ K562 (A1); HLA-A2⁺ K562 (A2); anti-CD3/CD28 coated beads (Beads; unspecific stimulation) or media alone (non-stimulated) before. Bars show mean values+SD. N=2 to 16 individual donors.

FIG. 18—Evaluation of Treg survival after unspecific and HLA.A2 specific activation in the absence of IL-2 normalised to relative CAR expression

FIG. 18 shows the survival ability of transduced CAR Tregs after co-culture in the presence of various stimuli in the absence of IL2; HLA-A1⁺ K562 (A1); HLA-A2⁺ K562 (A2); or media alone (non-stimulated). Percentage of live CD4⁺ CD25⁺ QBEND/10⁺ Treg populations are shown normalized to the relative MFI of A2⁺ Dextramer⁺ (CAR) expression. The percentage of survival derived from cultures of ‘media alone’ were pre-subtracted from the values displayed in the graphs. Bars show mean values. Dotted line shows mean value of percentage of survival of Con 1 Tregs cultured in the presence of HLA-A2⁺ K562. TRS-78, TRS-95 and TRS-96 are individual donor anonymised names. N=3.

FIG. 19—Evaluation of Treg survival after unspecific and HLA.A2 specific activation in the absence of IL-2 normalised to CD69 expression after HLA.A2 engagement

FIG. 19 shows the survival ability of transduced CAR Tregs after co-culture in the presence of various stimuli in the absence of IL2; HLA-A1⁺ K562 (A1); HLA-A2⁺ K562 (A2); or media alone (non-stimulated). Percentage of live CD4⁺ CD25⁺ QBEND/10⁺ Treg populations are shown normalized to the relative percentage of CD69 expression after HLA.A2 engagement. The percentage of survival derived from cultures of ‘media alone’ were pre-subtracted from the values displayed in the graphs. Bars show mean values. Dotted line shows mean value of percentage of survival of Control Tregs cultured in the presence of HLA-A2⁺ K562. TRS-95 and TRS-96 are individual donor anonymised names. N=2.

FIG. 20—STAT5 phosphorylation analysis as an indicator of IL2R CAR signaling (ELISA)

FIG. 20 shows the transduced CAR Treg levels of pSTAT5 after engagement with A2 beads as detected by ELISA using Optical Density (OD₄₅₀) measurements. The OD₄₅₀ readings derived from cultures of ‘media alone’ were pre-subtracted from the values displayed in the graphs. TRS-96 is the individual donor anonymised name. Bars show mean values+SD. This experiment was performed with technical duplicates. N=1.

FIG. 21—STAT5 phosphorylation analysis as an indicator of IL2R CAR signaling (Western Blot)

FIG. 21 shows levels of pSTAT5 protein of transduced CAR Tregs by Western blot. The graphs show normalized (to loading control) densitometric analysis of pSTAT5 protein levels measured by volume or percent of positive detection. The pSTAT5 protein background detection derived from cultures of ‘media alone’ were pre-subtracted from the values displayed in the graphs. TRS-96 is the individual donor anonymised name. Bars show mean values. N=1.

### DETAILED DESCRIPTION OF THE INVENTION

### Engineered Cell (e.g., Regulatory T Cell (Treg))

An “engineered cell” as used herein means a cell which has been modified to comprise or express a polynucleotide which is not naturally encoded by the cell, or in other words not naturally present in the cell, or wherein the expression product(s) of the polynucleotide are not naturally encoded by the cell. Methods for engineering cells are known in the art and include, but are not limited to, genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell. Non-viral technologies such as amphipathic cell penetrating peptides may be used to introduce nucleic acid in accordance with the present invention.

Accordingly, the polynucleotide encoding a chimeric receptor (e.g., a CAR) as described herein is not naturally expressed by a corresponding, unmodified cell. Suitably, an engineered cell is a cell which has been modified to introduce a polynucleotide into the cell, e.g. by transduction or by transfection. Suitably, an engineered cell is a cell which has been modified or whose genome has been modified e.g. by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells according to the present invention may be generated by introducing DNA or RNA encoding a chimeric receptor (e.g., a CAR) as described herein by one of many means including transduction with a viral vector, transfection with DNA or RNA. Cells may be activated and/or expanded prior to, or after, the introduction of a polynucleotide encoding the chimeric receptor (e.g., the CAR) as described herein, for example by treatment with an anti-CD3 monoclonal antibody or both anti-CD3 and anti-CD28 monoclonal antibodies. The Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

An engineered cell of the invention may be any cell, but particularly may be an immune cell, such as a T cell or an NK cell. Particularly, the cell may be a Tcon or a Treg. The cell may be a precursor for a Tcon or a Treg.

Alternatively, the engineered cell may be a pluripotent cell (such as an iPSC). A skilled person will appreciate that such engineered pluripotent cells will be capable of being differentiated into any cell type, and particularly may be differentiated into an immune cell, such as a T cell (e.g. a Treg), or an NK cell. Differentiation may occur through an intermediate cell group, for example, a pluripotent cell may be differentiated to a Tcon, which may be converted to a Treg. Such engineered pluripotent cells may be used as part of an allogeneic product strategy. iPSCs are commercially widely available for use and may be derived from somatic fibroblasts, CD34+ haematopoietic stem cells etc.

An engineered cell of the invention may be part of a population of cells. Suitably, the population of engineered cells comprises at least 70% engineered cells, such as at least 75, 85, 90, 95, 97, 98 or 99% engineered cells. Alternatively viewed, where the cell is an immune cell, a population of cells may comprise at least 70% immune cells, such as at least 75, 80, 85, 90, 95, 97, 98 or 99% immune cells (e.g., T cells or NK cells).

Regulatory T cells (Treg) are immune cells with immunosuppressive function that control cytopathic immune responses and are essential for the maintenance of immunological tolerance.

As used herein, the term Treg refers to a T cell with immunosuppressive function. A T cell as used herein is a lymphocyte including any type of T cell, such as an alpha beta T cell (e.g. CD8 or CD4+), a gamma delta T cell, a memory T cell, a Treg cell etc.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs of the present invention may proliferate 5%, 10%, 20%, 30%, 40%, 50%, 60%, 70%, 90%, 95% or 99% less than the same Tconv cells cultured in the absence of Tregs of the invention. For example, antigen-specific Tconv cells co-cultured with Tregs of the present invention may proliferate 5%, 10%, 20%, 30%, 40%, 50%, 60%, 70%, 90%, 95% or 99% less than the same Tconv cells cultured in the presence of non-engineered Tregs. The Tregs of the invention may have an increased suppressive activity as compared to non-engineered Tregs or to Tregs lacking exogenous FOXP3 but engineered to comprise an identical chimeric receptor (e.g., CAR) but which lacks a STAT5 association motif, a JAK1 and/or JAK2 binding motif and JAK3 binding motif within the endodomain (e.g. an increased suppressive activity of at least 5, 10, 20, 30, 40, 50, 60, 70, 80 or 90%).

Antigen-specific Tconv cells co-cultured with Tregs of the invention may express at least 10%, at least 20%, at least 30%, at least 40%, at least 50%, or at least 60% less effector cytokine than corresponding Tconv cells cultured in the absence of Tregs of the invention (e.g. in the presence of non-engineered Tregs, or Tregs lacking exogenous FOXP3 but engineered to comprise an identical chimeric receptor (e.g., CAR) but which lacks a STAT5 association motif, a JAK1 and/or JAK2 binding motif and JAK3 binding motif within the endodomain).

The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Suitably, the Treg is a T cell which expresses the markers CD4, CD25 and FOXP3 (CD4⁺ CD25⁺ FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Tregs may also express CTLA-4 (cytotoxic T-lymphocyte associated molecule-4) or GITR (glucocorticoid-induced TNF receptor). Treg cells are present in the peripheral blood, lymph nodes, and tissues and include thymus-derived, natural Treg (nTreg) cells and peripherally generated, induced Treg (iTreg) cells.

Suitably, the Treg may be identified using the cell surface markers CD4 and CD25 in the absence of or in combination with low-level expression of the surface protein CD127 (CD4⁺ CD25⁺ CD127⁻ or CD4⁺ CD25⁺ CD127^{low}). The use of such markers to identify Tregs is known in the art and described in Liu et al. (JEM; 2006; 203; 7(10); 1701-1711), for example.

The Treg may be a CD4⁺ CD25⁺ FOXP3⁺ T cell.

The Treg may be a CD4⁺ CD25⁺ CD127⁻ T cell.

The Treg may be a CD4⁺ CD25⁺ FOXP3⁺ CD127^{-/low} T cell.

The Treg may be natural or thymus-derived, adaptive or peripherally-derived, or in vitro-induced (Abbas, A. K., et al., 2013. Nature immunology, 14(4), p. 307-308).

Suitably, the Treg may be a natural Treg (nTreg). As used herein, the term “natural T reg” means a thymus-derived Treg. Natural T regs are CD4⁺ CD25⁺ FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Further suitable Tregs include, but are not limited to, Tr1 cells (which do not express Foxp3, and have high IL-10 production); CD8⁺ FOXP3⁺ T cells; and γδ FOXP3⁺ T cells.

Methods for determining the presence of cell markers are well-known in the art and include, for example, flow cytometry.

Suitably, the cell, such as a Treg, is isolated from peripheral blood mononuclear cells (PBMCs) obtained from a subject. Suitably the subject from whom the PBMCs are obtained is a mammal, preferably a human. Suitably the cell is matched (e.g. HLA matched) or is autologous to the subject to whom the engineered Treg is to be administered. Suitably, the subject to be treated is a mammal, preferably a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered Treg is to be administered.

Suitably, the Treg is isolated from peripheral blood mononuclear cells (PBMCs) obtained from a subject. In a preferred embodiment, the Treg is isolated from peripheral blood mononuclear cells (PBMCs) obtained from a subject and is matched or is autologous to the subject to be treated.

Alternatively, the Treg may be isolated from cord blood or from thymus, e.g., from paediatric thymus.

Suitably, the Treg is isolated from the subject to be treated.

Suitably, the Treg is part of a population of Tregs. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells (e.g. iPSCs) or embryonic progenitor cells to the Treg. A nucleic acid molecule or vector of the invention may be introduced into the inducible progenitor cells or embryonic progenitor cells prior to, or after, differentiation to a Treg. Suitable methods for differentiation are known in the art and include that disclosed in Haque et al, J Vis Exp., 2016, 117, 54720 (incorporated herein by reference).

As used herein, the term “conventional T cell” or Tcon or Tconv (used interchangeably herein) means a T lymphocyte cell which expresses an αβ T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may generated from a Tcon by introducing DNA or RNA coding for FOXP3 in addition to the DNA or RNA coding for the chimeric receptor (e.g., the CAR) as described herein, by one of many means including transduction with a viral vector, or transfection with DNA or RNA on the same or different vectors. Alternatively, the engineered Treg may be generated from a Tcon by in vitro culture of CD4+ CD25−FOXP3− cells in the presence of IL-2 and TGF-β.

A Treg of the invention may have increased persistence as compared to a Treg cell without exogenous FOXP3 but engineered to express the same chimeric receptor (e.g., CAR) but lacking a STAT5 association motif, a JAK1 and/or JAK2 binding motif and JAK3 binding motif within the endodomain. “Persistence” as used herein defines the length of time that Tregs can survive in a particular environment, e.g. in vivo (e.g. in a human patient or animal model). A Treg of the invention may have at least 10, 20, 30, 40, 50, 60, 70, 80 or 90% increased persistence as compared to a Treg engineered with a chimeric receptor (e.g., a CAR) but lacking a STAT5 association motif, a JAK1 binding motif and/or a JAK2 binding motif and a JAK3 binding motif in the endodomain and without exogenous FOXP3.

### FOXP3

In the present invention, FOXP3 expression is optionally increased in cells (e.g. Tregs) by introducing into the cells a nucleic acid molecule or vector as described herein encoding a FOXP3 polypeptide. The invention thus also provides a method for increasing FOXP3 in a cell, e.g. in a Treg or a CD4+ cell.

“FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

“Increasing FOXP3 expression” means to increase the levels of FOXP3 mRNA and/or protein in a cell (or population of cells) in comparison to a corresponding cell which has not been modified (or population of cells). For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Preferably the cell is a Treg or the population of cells is a population of Tregs. Suitably, the level of FOXP3 mRNA and/or protein in a cell modified by according to the present invention (or a population of such cells) may be increased to at least 1.5-fold greater, 2-fold greater, or 5-fold greater than the level in a corresponding cell which has not been modified according to the present invention (or population of such cells). Preferably the cell is a Treg or the population of cells is a population of Tregs.

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e. a polypeptide able to bind FOXP3 target DNA and function as a transcription factor regulating development and function of Tregs. Particularly, a FOXP3 polypeptide may have the same or similar activity to wildtype FOXP3 (SEQ ID NO. 52), e.g. may have at least 40, 50, 60, 70, 80, 90, 95, 100, 110, 120, 130, 140 or 150% of the activity of the wildtype FOXP3 polypeptide. Thus, a FOXP3 polypeptide encoded by the nucleic acid or vector described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference). As discussed in detail below, FOXP3 or a FOXP3 polypeptide includes functional fragments, variants, and isoforms thereof, e.g. of SEQ ID NO: 52.

A “functional fragment of FOXP3” may refer to a portion or region of a FOXP3 polypeptide or a polynucleotide encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp. 665-6′75; Lopes, J. E., et al., 2006. The Journal of Immunology, 177(5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO: 150 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide, e.g. to SEQ ID NO: 52. FOXP3 variants may have the same or similar activity to a wildtype FOXP3 polypeptide or polynucleotide, e.g. may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g. at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO: 52, for example S418E and/or S422A, as described in WO2019/241549 (incorporated herein by reference).

### Foxp3 Polypeptide Sequences

Suitably, the FOXP3 polypeptide encoded by a nucleic acid molecule or vector as described herein may comprise or consist of the polypeptide sequence of a human FOXP3, such as UniProtKB accession Q9BZS1 (SEQ ID NO: 52), or a functional fragment or variant thereof:

FOXP3, UniProtKB accession Q9BZS1 (SEQ ID NO: 52):
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKRSQRPSRCSNPTPGP

In some embodiments of the invention, the FOXP3 polypeptide comprises or consists of an amino acid sequence which is at least 70% identical to SEQ ID NO: 52 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO: 52 or a functional fragment thereof. In some embodiments, the FOXP3 polypeptide comprises or consists of SEQ ID NO: 52 or a functional fragment thereof.

In some embodiments, as discussed above, the FOXP3 polypeptide may comprise mutations at residues 418 and/or 422 of SEQ ID NO: 52, as follows:

(SEQ ID NO: 147)
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKREQRPSRCSNPTPGP;
(SEQ ID NO: 148)
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKRSQRPARCSNPTPGP;
or
(SEQ ID NO: 149)
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKREQRPARCSNPTPGP.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of or a functional variant thereof having at least 80, 85, 90, 95 or 99% identity thereto:

(SEQ ID NO: 150)
GGAHASSSSL NPMPPSQLQL PTLPLVMVAP SGARLGPLPH
LQALLQDRPH FMHQLSTVDA HARTPVLQVH PLESPAMISL
TPPTTATGVF SLKARPGLPP GINVASLEWV SREPALLCTF
PNPSAPRKDS TLSAVPQSSY PLLANGVCKW PGCEKVFEEP
EDFLKHCQAD HLLDEKGRAQ CLLQREMVQS LEQQLVLEKE
KLSAMQAHLA GKMALTKASS VASSDKGSCC IVAAGSQGPV
VPAWSGPREA PDSLFAVRRH LWGSHGNSTF PEFLHNMDYF
KFHNMRPPFT YATLIRWAIL EAPEKQRTLN EIYHWFTRMF
AFFRNHPATW KNAIRHNLSL HKCFVRVESE KGAVWTVDEL
EF

Suitably, the FOXP3 polypeptide may be a variant of SEQ ID NO: 52, for example a natural variant. Suitably, the FOXP3 polypeptide is an isoform of SEQ ID NO: 52. For example, the FOXP3 polypeptide may comprise a deletion of amino acid positions 72-106 relative to SEQ ID NO: 52. Alternatively, the FOXP3 polypeptide may comprise a deletion of amino acid positions 246-272 relative to SEQ ID NO: 52.

Suitably, the FOXP3 polypeptide comprises SEQ ID NO: 151 or a functional fragment thereof:

Illustrative FOXP3 polypeptide (SEQ ID NO: 151):
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQVEELSA
MQAHLAGKMALTKASSVASSDKGSCCIVAAGSQGPVVPAWSGPREAPDSL
FAVRRHLWGSHGNSTFPEFLHNMDYFKFHNMRPPFTYATLIRWAILEAPE
KQRTLNEIYHWFTRMFAFFRNHPATWKNAIRHNLSLHKCFVRVESEKGAV
WTVDELEFRKKRSQRPSRCSNPTPGPEGRGSLLTCGDVEEN

Suitably the FOXP3 polypeptide comprises or consists of an amino acid sequence which is at least 70% identical to SEQ ID NO: 151 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 151 or a functional fragment thereof. In some embodiments, the FOXP3 polypeptide comprises or consists of SEQ ID NO: 151 or a functional fragment thereof.

Suitably, the FOXP3 polypeptide may be a variant of SEQ ID NO: 151, for example a natural variant. Suitably, the FOXP3 polypeptide is an isoform of SEQ ID NO: 151 or a functional fragment thereof. For example, the FOXP3 polypeptide may comprise a deletion of amino acid positions 72-106 relative to SEQ ID NO: 151. Alternatively, the FOXP3 polypeptide may comprise a deletion of amino acid positions 246-272 relative to SEQ ID NO: 151.

### Foxp3 Polynucleotide Sequences

Suitably, the polynucleotide encoding a FOXP3 polypeptide comprises or consists of a polynucleotide sequence set forth in SEQ ID NO: 152:

Illustrative FOXP3 polynucleotide (SEQ ID NO: 152):
(SEQ ID NO: 152)
ATGCCCAACCCCAGGCCTGGCAAGCCCTCGGCCCCTTCCTTGGCCCTTGG
CCCATCCCCAGGAGCCTCGCCCAGCTGGAGGGCTGCACCCAAAGCCTCAG
ACCTGCTGGGGGCCCGGGGCCCAGGGGGAACCTTCCAGGGCCGAGATCTT
CGAGGCGGGGCCCATGCCTCCTCTTCTTCCTTGAACCCCATGCCACCATC
GCAGCTGCAGCTGCCCACACTGCCCCTAGTCATGGTGGCACCCTCCGGGG
CACGGCTGGGCCCCTTGCCCCACTTACAGGCACTCCTCCAGGACAGGCCA
CATTTCATGCACCAGCTCTCAACGGTGGATGCCCACGCCCGGACCCCTGT
GCTGCAGGTGCACCCCCTGGAGAGCCCAGCCATGATCAGCCTCACACCAC
CCACCACCGCCACTGGGGTCTTCTCCCTCAAGGCCCGGCCTGGCCTCCCA
CCTGGGATCAACGTGGCCAGCCTGGAATGGGTGTCCAGGGAGCCGGCACT
GCTCTGCACCTTCCCAAATCCCAGTGCACCCAGGAAGGACAGCACCCTTT
CGGCTGTGCCCCAGAGCTCCTACCCACTGCTGGCAAATGGTGTCTGCAAG
TGGCCCGGATGTGAGAAGGTCTTCGAAGAGCCAGAGGACTTCCTCAAGCA
CTGCCAGGCGGACCATCTTCTGGATGAGAAGGGCAGGGCACAATGTCTCC
TCCAGAGAGAGATGGTACAGTCTCTGGAGCAGCAGCTGGTGCTGGAGAAG
GAGAAGCTGAGTGCCATGCAGGCCCACCTGGCTGGGAAAATGGCACTGAC
CAAGGCTTCATCTGTGGCATCATCCGACAAGGGCTCCTGCTGCATCGTAG
CTGCTGGCAGCCAAGGCCCTGTCGTCCCAGCCTGGTCTGGCCCCCGGGAG
GCCCCTGACAGCCTGTTTGCTGTCCGGAGGCACCTGTGGGGTAGCCATGG
AAACAGCACATTCCCAGAGTTCCTCCACAACATGGACTACTTCAAGTTCC
ACAACATGCGACCCCCTTTCACCTACGCCACGCTCATCCGCTGGGCCATC
CTGGAGGCTCCAGAGAAGCAGCGGACACTCAATGAGATCTACCACTGGTT
CACACGCATGTTTGCCTTCTTCAGAAACCATCCTGCCACCTGGAAGAACG
CCATCCGCCACAACCTGAGTCTGCACAAGTGCTTTGTGCGGGTGGAGAGC
GAGAAGGGGGCTGTGTGGACCGTGGATGAGCTGGAGTTCCGCAAGAAACG
GAGCCAGAGGCCCAGCAGGTGTTCCAACCCTACACCTGGCCCCTGA

In some embodiments of the invention, the polynucleotide encoding the FOXP3 polypeptide or variant comprises a polynucleotide sequence which is at least 70% identical to SEQ ID NO: 152 or a functional fragment thereof. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 152 or a functional fragment thereof. In some embodiments of the invention, the polynucleotide encoding the FOXP3 polypeptide or variant comprises or consists of SEQ ID NO: 152 or a functional fragment thereof.

Suitably, the polynucleotide encoding a FOXP3 polypeptide comprises or consists of a polynucleotide sequence set forth in SEQ ID NO: 153:

Illustrative FOXP3 polynucleotide (SEQ ID NO: 153):
(SEQ ID NO: 153)
GAATTCGTCGACATGCCCAACCCCAGACCCGGCAAGCCTTCTGCCCCTTC
TCTGGCCCTGGGACCATCTCCTGGCGCCTCCCCATCTTGGAGAGCCGCCC
CTAAAGCCAGCGATCTGCTGGGAGCTAGAGGCCCTGGCGGCACATTCCAG
GGCAGAGATCTGAGAGGCGGAGCCCACGCCTCTAGCAGCAGCCTGAATCC
CATGCCCCCTAGCCAGCTGCAGCTGCCTACACTGCCTCTCGTGATGGTGG
CCCCTAGCGGAGCTAGACTGGGCCCTCTGCCTCATCTGCAGGCTCTGCTG
CAGGACCGGCCCCACTTTATGCACCAGCTGAGCACCGTGGACGCCCACGC
CAGAACACCTGTGCTGCAGGTGCACCCCCTGGAAAGCCCTGCCATGATCA
GCCTGACCCCTCCAACCACAGCCACCGGCGTGTTCAGCCTGAAGGCCAGA
CCTGGACTGCCCCCTGGCATCAATGTGGCCAGCCTGGAATGGGTGTCCCG
CGAACCTGCCCTGCTGTGCACCTTCCCCAATCCTAGCGCCCCCAGAAAGG
ACAGCACACTGTCTGCCGTGCCCCAGAGCAGCTATCCCCTGCTGGCTAAC
GGCGTGTGCAAGTGGCCTGGCTGCGAGAAGGTGTTCGAGGAACCCGAGGA
CTTCCTGAAGCACTGCCAGGCCGACCATCTGCTGGACGAGAAAGGCAGAG
CCCAGTGCCTGCTGCAGCGCGAGATGGTGCAGTCCCTGGAACAGCAGCTG
GTGCTGGAAAAAGAAAAGCTGAGCGCCATGCAGGCCCACCTGGCCGGAAA
GATGGCCCTGACAAAAGCCAGCAGCGTGGCCAGCTCCGACAAGGGCAGCT
GTTGTATCGTGGCCGCTGGCAGCCAGGGACCTGTGGTGCCTGCTTGGAGC
GGACCTAGAGAGGCCCCCGATAGCCTGTTTGCCGTGCGGAGACACCTGTG
GGGCAGCCACGGCAACTCTACCTTCCCCGAGTTCCTGCACAACATGGACT
ACTTCAAGTTCCACAACATGAGGCCCCCCTTCACCTACGCCACCCTGATC
AGATGGGCCATTCTGGAAGCCCCCGAGAAGCAGCGGACCCTGAACGAGAT
CTACCACTGGTTTACCCGGATGTTCGCCTTCTTCCGGAACCACCCCGCCA
CCTGGAAGAACGCCATCCGGCACAATCTGAGCCTGCACAAGTGCTTCGTG
CGGGTGGAAAGCGAGAAGGGCGCCGTGTGGACAGTGGACGAGCTGGAATT
TCGGAAGAAGCGGTCCCAGAGGCCCAGCCGGTGTAGCAATCCTACACCTG
GCCCTGAGGGCAGAGGAAGTCTGCTAACATGCGGTGACGTCGAGGAGAAT
CC.

In some embodiments of the invention, the polynucleotide encoding the FOXP3 polypeptide or variant comprises a polynucleotide sequence which is at least 70% identical to SEQ ID NO: 153 or a functional fragment thereof. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 153 or a functional fragment thereof. In some embodiments of the invention, the polynucleotide encoding the FOXP3 polypeptide or variant comprises or consists of SEQ ID NO: 153 or a functional fragment thereof.

Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

### Chimeric Receptor (Cr)

“Chimeric receptor” or “CR”, as used herein refers to engineered receptors which can confer a binding specificity onto cells. Chimeric receptors may bind to ligand (typically a biological molecule), particularly to an antigen or to another biological molecule (for example, to a cytokine or any other soluble molecule). Typically, therefore, the chimeric receptor of the invention may comprise a ligand binding domain (particularly, a ligand binding exodomain), which is capable of binding to a ligand.

Chimeric receptors as referred to herein, particularly include CARs which confer antigen specificity to a cell as discussed in detail below.

Chimeric receptors of the invention may be capable of binding to any biological molecule and thus the ligand binding domain may include any naturally occurring, synthetic, semi-synthetic, or recombinantly produced binding partner for a biological molecule of interest (e.g., which bind to ligands such as antigens, biological receptors, antibodies (e.g., Rituximab, anti-CD34 antibodies), cytokines (for example, IL10, IL7, IL15, IL33), chemokines (for example, CXC 12, CCL2, CCL4, CCL5 and CCL22), secreted factors (e.g., tumour secreted factors, for example, prostate-specific antigen (PSA), carcinoembryonic antigen (CEA), vascular endothelial growth factor (VEGF) and CA125), etc).

The ligand binding domains of chimeric receptors are well documented and examples can for example, be found in WO2012/138858, WO2017/029512 etc, which are incorporated by reference. Further discussion and examples of ligand binding domains can be found below in the detailed discussion of chimeric antigen receptors, including antibody-based, and receptor-based ligand binding domains.

In a particular embodiment, the chimeric receptor defined herein does not comprise a first and a second heterodimerisation domain. The first and second heterodimerisation domains (Ht1 and HT2, respectively) allow, in the presence of a chemical inducer of dimerization (CID), interaction of an identical pair of the chimeric receptors, such that Ht1 from one chimeric receptor heterodimerises with Ht2 from the other chimeric receptor, causing dimerization of the two signalling domains, particularly such that upon said dimerization, STAT5-mediated signalling is induced. Thus, the chimeric receptor of the invention may not comprise a pair of cognate hetero-dimerisable domains.

Typically, a chimeric receptor of the invention comprises an extracellular ligand binding domain, a transmembrane domain, optionally, one or more co-stimulatory domains, and an intracellular signalling domain (also referred to as an endodomain). Particularly, as discussed in detail below, the endodomain of the chimeric receptor of the invention comprises a JAK3 binding motif, and a STAT5 association motif and a JAK1 and/or JAK2 binding motif, allowing the delivery of an IL2 persistence signal into the cell upon ligand binding. The chimeric receptor of the invention is particularly provided as a single continuous chain.

Chimeric receptor encoding polynucleotides may be transferred to a cell using, for example, retroviral vectors, thereby generating large numbers of engineered cells for therapy. When the chimeric receptor binds to its ligand, a persistence/survival signal is transmitted to the cell in which it is expressed. Thus, a chimeric receptor of the invention allows the provision of a persistence signal to a cell.

### Intracellular Signaling Domain (Endodomain)

The present chimeric receptor (e.g., CAR) comprises an endodomain which comprises a STAT5 association motif, a JAK1 and/or a JAK2 binding motif, and a JAK3 binding motif

“Signal Transducer and Activator of Transcription 5” (STAT5) is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation results in STAT5 phosphorylation by activating the Jak1/Jak2 and Jak3 kinases via specific signalling domains present in the IL-2R13 and IL-2Ry chain, respectively. Although Jak1 (or Jak2) can phosphorylate STAT5 without the need of Jak3, STAT5 activity is increased by the transphosphorylation of both Jak1/Jak2 and Jak3, which stabilizes their activity.

“STAT5 association motif′ as used herein refers to an amino acid motif which comprises a tyrosine and is capable of binding a STAT5 polypeptide. Any method known in the art for determining protein:protein interactions may be used to determine whether an association motif is capable of binding to STAT5. For example, co-immunoprecipitation followed by western blot.

Suitably, the chimeric receptor (e.g., CAR) endodomain may comprise two (e.g. at least two) or more STAT5 association motifs as defined herein. For example, the chimeric receptor (e.g., CAR) endodomain may comprise two, three, four, five or more STAT5 association motifs as defined herein. Preferably, the chimeric receptor (e.g., CAR) endodomain may comprise two or three STAT5 association motifs as defined herein.

Suitably, the STAT5 association motif may exist endogenously in a cytoplasmic domain of a transmembrane protein. For example, the STAT5 association motif may be from an interleukin receptor (IL) receptor endodomain or a hormone receptor.

The chimeric receptor (e.g., CAR) endodomain may comprise an amino acid sequence selected from any chain of the interleukin receptors where STAT5 is a downstream component, for example, the cytoplasmic domain comprising amino acid numbers 266 to 551 of IL-2 receptor β chain (NCBI REFSEQ: NP 000869.1, SEQ ID NO: 1), amino acid numbers 265 to 459 of IL-7R a chain (NCBI REFSEQ: NP 002176.2, SEQ ID NO: 2), amino acid numbers 292 to 521 of IL-9R chain (NCBI REFSEQ: NP 002177.2, SEQ ID NO: 3), amino acid numbers 257 to 825 of IL-4R a chain (NCBI REFSEQ: NPJD00409.1, SEQ ID NO: 4), amino acid numbers 461 to 897 of IL-3R β chain (NCBI REFSEQ: NP 000386.1, SEQ ID NO: 5) and/or amino acid numbers 314 to 502 of IL-17R β chain (NCBI REFSEQ: NP 061195.2, SEQ ID NO: 6) may be used. It will be appreciated by a skilled person that any one or more of these sequences can be used. The entire region of the cytoplasmic domain of interleukin receptor chain may be used.

IL7RA (AA 265 to 459 of NP_002176.2)
SEQ ID NO: 2
KKRIKPIVWPSLPDHKKTLEHLCKKPRKNLNVSFNPESFLDCQIHRVDDI
QARDEVEGFLQDTFPQQLEESEKQRLGGDVQSPNCPSEDVVITPESFGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNQ
IL7RA 2Y truncated:
SEQ ID NO: 7
KKRIKPIVWPSLPDHKKTLEHLCKKPRKNLNVSFNPESFLDCQIHRVDDI
QARDEVEGFLQDTFPQQPILTSLGSNQEEAYVTMSSFYQNQ
IL9R (AA 292 to 521 of NP_002177.2)
SEQ ID NO: 3
KLSPRVKRIFYQNVPSPAMFFQPLYSVHNGNFQTWMGAHGAGVLLSQDCA
GTPQGALEPCVQEATALLTCGPARPWKSVALEEEQEGPGTRLPGNLSSED
VLPAGCTEWRVQTLAYLPQEDWAPTSLTRPAPPDSEGSRSSSSSSSSNNN
NYCALGCYGGWHLSALPGNTQSSGPIPALACGLSCDHQGLETQQGVAWVL
AGHCQRPGLHEDLQGMLLPSVLSKARSWTF
IL4RA (AA 257 to 825 of NPJD00409.1)
SEQ ID NO: 4
KIKKEWWDQIPNPARSRLVAIIIQDAQGSQWEKRSRGQEPAKCPHWKNCL
TKLLPCFLEHNMKRDEDPHKAAKEMPFQGSGKSAWCPVEISKTVLWPESI
SVVRCVELFEAPVECEEEEEVEEEKGSFCASPESSRDDFQEGREGIVARL
TESLFLDLLGEENGGFCQQDMGESCLLPPSGSTSAHMPWDEFPSAGPKEA
PPWGKEQPLHLEPSPPASPTQSPDNLTCTETPLVIAGNPAYRSFSNSLSQ
SPCPRELGPDPLLARHLEEVEPEMPCVPQLSEPTTVPQPEPETWEQILRR
NVLQHGAAAAPVSAPTSGYQEFVHAVEQGGTQASAVVGLGPPGEAGYKAF
SSLLASSAVSPEKCGFGASSGEEGYKPFQDLIPGCPGDPAPVPVPLFTFG
LDREPPRSPQSSHLPSSSPEHLGLEPGEKVEDMPKPPLPQEQATDPLVDS
LGSGIVYSALTCHLCGHLKQCHGQEDGGQTPVMASPCCGCCCGDRSSPPT
TPLRAPDPSPGGVPLEASLCPASLAPSGISEKSKSSSSFHPAPGNAQSSS
QTPKIVNFVSVGPTYMRVS
IL3RB (AA 461 to 897 of NP_000386.1)
SEQ ID NO: 5
RFCGIYGYRLRRKWEEKIPNPSKSHLFQNGSAELWPPGSMSAFTSGSPPH
QGPWGSRFPELEGVFPVGFGDSEVSPLTIEDPKHVCDPPSGPDTTPAASD
LPTEQPPSPQPGPPAASHTPEKQASSFDFNGPYLGPPHSRSLPDILGQPE
PPQEGGSQKSPPPGSLEYLCLPAGGQVQLVPLAQAMGPGQAVEVERRPSQ
GAAGSPSLESGGGPAPPALGPRVGGQDQKDSPVAIPMSSGDTEDPGVASG
YVSSADLVFTPNSGASSVSLVPSLGLPSDQTPSLCPGLASGPPGAPGPVK
SGFEGYVELPPIEGRSPRSPRNNPVPPEAKSPVLNPGERPADVSPTSPQP
EGLLVLQQVGDYCFLPGLGPGPLSLRSKPSSPGPGPEIKNLDQAFQVKKP
PGQAVPQVPVIQLFKALKQQDYLSLPPWEVNKPGEVC
IL17RB (AA 314 to 502 of NP_061195.2)
SEQ ID NO: 6
RHERIKKTSFSTTTLLPPIKVLVVYPSEICFHHTICYFTEFLQNHCRSEV
ILEKWQKKKIAEMGPVQWLATQKKAADKVVFLLSNDVNSVCDGTCGKSEG
SPSENSQDLFPLAFNLFCSDLRSQIHLHKYVVVYFREIDTKDDYNALSVC
PKYHLMKDATAFCAELLHVKQQVSAGKRSQACHDGCCSL

The chimeric receptor (e.g., CAR) endodomain may comprise one or more STAT5 association motifs that comprise an amino acid sequence shown as SEQ ID NO: 1-7, or a variant which is at least 80, 85, 90, 95, 96, 97, 98 or 99% identical to SEQ ID NO: 1-7. For example, the variant may be capable of binding STAT5 to at least 10%, at least 20%, at least 30%, at least 40%, at least 50%, at least 60%, at least 70%, at least 80%, or at least 90% of the level of an amino acid sequence shown as one of SEQ ID NO: 1-7. The variant or derivative may be capable of binding STAT5 to a similar or the same level as one of SEQ ID NO: 1-7 or may be capable of binding STAT5 to a greater level than an amino acid sequence shown as one of SEQ ID NO: 1-7 (e.g. increased by at least 10%, at least 20%, at least 30%, at least 40% or at least 50%).

For example, the STAT5 association motif may be from any one or more of IL2Rβ, IL7Ra, IL-3Rβ (CSF2RB), IL-9R, IL-17R13, erythropoietin receptor, thrombopoietin receptor, growth hormone receptor and prolactin receptor. A chimeric receptor (e.g., CAR) may, for example, comprise STAT association motifs from both IL2Rβ and IL7Rα.

The STAT5 association motif may comprise the amino acid motif YXXF/L (SEQ ID NO: 8); wherein X is any amino acid.

Suitably, the STAT5 association motif may comprise the amino acid motif YCTF (SEQ ID NO: 9), YFFF (SEQ ID NO: 10), YLSL (SEQ ID NO: 11), or YLSLQ (SEQ ID NO: 12).

Suitably, the STAT5 association motif may comprise the amino acid motif YLSLQ (SEQ ID NO: 12).

The chimeric receptor (e.g., CAR) endodomain may comprise one or more STAT5 association motif comprising the amino acid motif YCTF (SEQ ID NO: 9), YFFF (SEQ ID NO: 10), YLSL (SEQ ID NO: 11), and/or YLSLQ (SEQ ID NO: 12).

The chimeric receptor (e.g., CAR) endodomain may comprise a first STAT5 association motif comprising the amino acid motif YLSLQ (SEQ ID NO: 12) and a second STAT5 association motif comprising the amino acid motif YCTF (SEQ ID NO: 9) or YFFF (SEQ ID NO: 10).

The chimeric receptor (e.g., CAR) endodomain may comprise the following STAT5 association motifs: YLSLQ (SEQ ID NO: 12), YCTF (SEQ ID NO: 9) and YFFF (SEQ ID NO: 10).

“JAK1 and/or a JAK2 binding motif′ as used herein refers to BOX motif which allows for tyrosine kinase JAK1 and/or JAK2 association. Suitable JAK1 and JAK2 binding motifs are described, for example, by Ferrao & Lupardus (Frontiers in Endocrinology; 2017; 8 (71); which is incorporated herein by reference).

The JAK1 and/or JAK2 binding motif may occur endogenously in a cytoplasmic domain of a transmembrane protein.

For example, the JAK1 and/or JAK2 binding motif may be from Interferon lambda receptor 1 (IFNLR1), Interferon alpha receptor 1 (IFNAR), Interferon gamma receptor 1 (IFNGR1), IL10RA, IL20RA, IL22RA, Interferon gamma receptor 2 (IFNGR2) or IL10RB.

The JAK1 binding motif may comprise or consist of an amino acid motif shown as SEQ ID NO: 13-19 or a variant therefore which is capable of binding JAK1.

(SEQ ID NO: 13)
KVLKCNTPDPSKFFSQLSSEHGGDVQKWLSSPFPSSSFSPGGLAP
EISPLEVLERDK
(SEQ ID NO: 14)
NPWFQRAKMPRALDFSGHTHPVATFQPSRPESVNDLFLCPQKELT
(SEQ ID NO: 15)
GYICLRNSLPKVLNFHNFLAWPFPNLPPLEAMDMVEVIYINR
(SEQ ID NO: 16)
PLKEKSIILPKSLISVVRSATLETKPESKYVSLITSYQPFSL
(SEQ ID NO: 17)
RRRKKLPSVLLFKKPSPFIFISQRPSPETQDTIHPLDEEAFLK
(SEQ ID NO: 18)
YIHVGKEKHPANLILIYGNEFDKRFFVPAEKIVINFITLNISDDS
(SEQ ID NO: 19)
RYVTKPPAPPNSLNVQRVLTFQPLRFIQEHVLIPVFDLSGP

The variant of SEQ ID NO: 13-19 may comprise one, two or three amino acid differences compared to any of SEQ ID NO: 13-19 and retain the ability to bind JAK1.

The variant may be at least 80, 85, 90, 95, 96, 97, 98 or 99% identical to any one of SEQ ID NO: 13-19 and retain the ability to bind JAK1.

In a preferred embodiment, the JAK1 binding domain comprises or consists of SEQ ID NO: 13 or a variant thereof which is capable of binding JAK1.

For example, the variant may be capable of binding JAK1 to at least 10%, at least 20%, at least 30%, at least 40%, at least 50%, at least 60%, at least 70%, at least 80%, or at least 90% of the level of a corresponding, reference sequence. The variant or derivative may be capable of binding JAK1 to a similar or the same level as a corresponding, reference sequence or may be capable of binding JAK1 to a greater level than a corresponding, reference sequence (e.g. increased by at least 10%, at least 20%, at least 30%, at least 40% or at least 50%).

The JAK2 binding motif may comprise or consist of an amino acid motif shown as SEQ ID NO: 20-22 or a variant therefore which is capable of binding JAK2.

(SEQ ID NO: 20)
NYVFFPSLKPSSSIDEYFSEQPLKNLLLSTSEEQIEKCFIIEN
(SEQ ID NO: 21)
YWFHTPPSIPLQIEEYLKDPTQPILEALDKDSSPKDDVWDSVSIISFPE
(SEQ ID NO: 22)
YAFSPRNSLPQHLKEFLGHPHHNTLLFFSFPLSDENDVFDKLSVIAEDSES

The variant of SEQ ID NO: 21-22 may comprise one, two or three amino acid differences compared to any of SEQ ID NO: 20-22 and retain the ability to bind JAK2.

For example, the variant may be capable of binding JAK2 to at least 10%, at least 20%, at least 30%, at least 40%, at least 50%, at least 60%, at least 70%, at least 80%, or at least 90% of the level of a corresponding, reference sequence. The variant or derivative may be capable of binding JAK2 to a similar or the same level as a corresponding, reference sequence or may be capable of binding JAK2 to a greater level than a corresponding, reference sequence (e.g. increased by at least 10%, at least 20%, at least 30%, at least 40% or at least 50%).

Any method known in the art for determining protein:protein interactions may be used to determine whether a JAK1 or JAK2 binding motif is capable of binding to a JAK1 or JAK2. For example, co-immunoprecipitation followed by western blot

Suitably, the chimeric receptor (e.g., CAR) endodomain may comprise an IL2Rβ endodomain shown as SEQ ID NO: 1; or a variant which has at least 80% sequence identity to SEQ ID NO: 1.

SEQ ID NO: 1
NCRNTGPWLKKVLKCNTPDPSKFFSQLSSEHGGDVQKWLSSPFPSSSF
SPGGLAPEISPLEVLERDKVTQLLLQQDKVPEPASLSSNHSLTSCFTN
QGYFFFHLPDALEIEACQVYFTYDPYSEEDPDEGVAGAPTGSSPQPLQ
PLSGEDDAYCTFPSRDDLLLFSPSLLGGPSPPSTAPGGSGAGEERMPP
SLQERVPRDWDPQPLGPPTPGVPDLVDFQPPPELVLREAGEEVPDAGP
REGVSFPWSRPPGQGEFRALNARLPLNTDAYLSLQELQGQDPTHLV

The variant may be at least 80, 85, 90, 95, 96, 97, 98 or 99% identical to SEQ ID NO: 1.

Suitably, the chimeric receptor (e.g., CAR) endodomain may comprise a truncated IL2Rβ endodomain shown as any one of SEQ ID NO: 23 or 24; or a variant of any one of SEQ ID NO: 23 or 24 which has at least 80% sequence identity thereto.

(IL2RB truncated-Y510)
SEQ ID NO: 23
NCRNTGPWLKKVLKCNTPDPSKFFSQLSSEHGGDVQKWLSSPFPSSSF
SPGGLAPEISPLEVLERDKVTQLLPLNTDAYLSLQELQGQDPTHLV
(IL2RB truncated-Y510 & Y392)
SEQ ID NO: 24
NCRNTGPWLKKVLKCNTPDPSKFFSQLSSEHGGDVQKWLSSPFPSSSF
SPGGLAPEISPLEVLERDKVTQLLDAYCTFPSRDDLLLFSPSLLGGPS
PPSTAPGGSGAGEERMPPSLQERVPRDWDPQPLGPPTPGVPDLVDFQP
PPELVLREAGEEVPDAGPREGVSFPWSRPPGQGEFRALNARLPLNTDA
YLSLQELQGQDPTHLV

The variant may be at least 80, 85, 90, 95, 96, 97, 98 or 99% identical to SEQ ID NO: 23 or 24.

STAT5 activity is increased by the transphosphorylation of both a Jak1/2 and Jak3, as this stabilizes their activity. Suitably, the chimeric receptor (e.g., CAR) endodomain as described herein further comprises a JAK3 binding motif “JAK3 binding motif′ as used herein refers to BOX motif which allows for tyrosine kinase JAK3. Suitable JAK3 binding motifs are described, for example, by Ferrao & Lupardus (Frontiers in Endocrinology; 2017; 8 (71); which is incorporated herein by reference).

Any method known in the art for determining protein:protein interactions may be used to determine whether a motif is capable of binding to JAK3. For example, co-immunoprecipitation followed by western blot.

The JAK3 binding motif may occur endogenously in a cytoplasmic domain of a transmembrane protein.

For example, the JAK3 binding motif may be from an IL-2Ry polypeptide. A functional truncated or variant IL2Ry polypeptide may be used within the endodomain of the chimeric receptors (e.g., CARs) described herein, wherein the functional truncated or variant IL2Ry polypeptide retains JAK3 binding activity (e.g. at least 20, 30, 40, 50, 60, 70, 80, 90 or 95% of binding activity of IL2Ry). Particularly, a truncated IL2Ry comprising a JAK3 binding motif and a truncated IL2Rβ comprising a STAT5 association motif, and a JAK1 and/or JAK2 binding motif are comprised in the endodomain of a chimeric receptor (e.g., CAR) as defined herein. Functional truncations may provide an advantage of reducing construct size for expression.

The JAK3 binding motif may comprise or consist of an amino acid motif shown as SEQ ID NO: 25 or SEQ ID NO: 26 or a variant therefore which is capable of binding JAK3 (e.g. a functional variant or fragment having at least 80, 85, 90, 95 or 99% identity to SEQ ID NOs 25 or 26).

SEQ ID NO: 25
ERTMPRIPTLKNLEDLVTEYHGNFSAWSGVSKGLAESLQPDYSERLCL
VSEI
SEQ ID NO: 26
ERTMPRIPTLKNLEDLVTEYHGNFSAWSGVSKGLAESLQPDYSERLCL
VSEIPPKGGALGEGPGASPCNQHSPYWAPPCYTLKPET

The variant may be at least 80, 85, 90, 95, 96, 97, 98 or 99% identical to SEQ ID NO: 25 or SEQ ID NO: 26.

In a preferred embodiment, the chimeric receptor (e.g., CAR) endodomain comprises one or more JAKI binding domains and at least one JAK3 binding domain/motif (e.g. at least 2 or 3 JAK3 binding domains/motifs).

It will be appreciated by a skilled person that the polynucleotide sequence encoding the JAK3 binding domain may be positioned upstream or downstream (5′ or 3′) of the polynucleotide sequence encoding the STAT5 association motif and JAKI and/or JAK2 binding motifs. Particularly, the polynucleotide encoding the JAK3 binding domain may be positioned upstream (5′) of the polynucleotide encoding the STAT5 association motif and the JAKI and/or JAK2 binding motifs. Thus, alternatively viewed, in the chimeric receptor (e.g., CAR) as described herein, the JAK3 binding domain may be N or C terminal to the STAT5 association motif and the JAKI and/or JAK2 binding domains. In one embodiment, the JAK3 binding domain and the STAT5 association motif/JAK1 and/or JAK2 binding domains are positioned directly adjacent to one another (i.e. are not separated distally by sequence).

Further, a skilled person will appreciate that the JAK3 binding domain and the STAT5 association motif and JAKI and/or JAK2 binding domains, may be positioned at any location within the cytoplasmic domain or endodomain of the chimeric receptor (e.g., CAR), e.g. proximal to the membrane, or separated from the membrane by additional sequence, e.g. by one or more costimulatory domains. In one embodiment, it is possible for the domains to extend into the transmembrane region.

The various domains of the chimeric receptor, and individual parts of the domains (e.g. the motifs in the signalling domain) may be linked to one another by linkers. Thus, the chimeric receptor may contain one or more linkers. Typically, it will contain at least one linker. A linker as referred to herein is an amino acid sequence which links one domain or part of the protein to another. The linker sequence may be any amino acid sequence which functions to link, or connect, two domains or parts thereof together, such that they may perform their function. Thus, a linker may space apart the elements which are linked.

In a particular embodiment, a linker or a hinge may be present between the JAK3 binding motif and the STAT5 association motif/JAK1 and/or JAK2 binding motifs. Indeed, the inventors have identified that the presence of a linker between the JAK3 binding motif and the STAT5 association motif/JAK1 and/or JAK2 binding motifs in the endodomain of a chimeric receptor results in increased survival in cells expressing the chimeric receptor as compared to cells expressing a chimeric receptor having an endodomain comprising a JAK3 binding motif and a STAT5 association motif/JAK1 and/or JAK2 binding motifs without the linker. The increase in survival may be an increase of at least 10, 20, 30, 40, 50, 60, 70, 80 or 90% and can be measured by determining numbers of alive transduced cells after culture for a particular time period (particularly culture under low IL-2 conditions).

The nature of the linker, in terms of its amino acid composition and/or sequence of amino acids may be varied and is not limited. However, the linker may be a flexible linker. It may thus comprise or consist of amino acids known to confer a flexible character to the linker (as opposed to a rigid linker).

Flexible linkers are a category of linker sequences well known and described in the art. Linker sequences are generally known as sequences which may be used to link, or join together, proteins or protein domains, to create for example fusion proteins or chimeric proteins, or multifunctional proteins or polypeptides. They can have different characteristics, and for example may be flexible, rigid or cleavable. Protein linkers are reviewed for example in Chen et al., 2013, Advanced Drug Delivery Reviews 65, 1357-1369, which compares the category of flexible linkers with those of rigid and cleavable linkers. Flexible linkers are also described in Klein et al., 2014, Protein Engineering Design and Selection, 27(10), 325-330; van Rosmalen et al., 2017, Biochemistry, 56, 6565-6574; and Chichili et al., 2013, Protein Science, 22, 153-167.

A flexible linker is a linker which allows a degree of movement between the domains, or components, which are linked. They are generally composed of small non-polar (e.g. Gly) or polar (e.g. Ser or Thr) amino acid residues. The small size of the amino acids provides flexibility and allows for mobility of the connected parts (domains or components). The incorporation of polar amino acids can maintain the stability of the linker in aqueous environments by forming hydrogen bonds with water molecules.

The most commonly used flexible linkers have sequences primarily composed of Ser and Gly residues (so-called “GS linkers”). However, many other flexible linkers have also been described (see Chen et al, 2013, supra, for example), which may contain additional amino acids such as Thr and/or Ala, and/or Lys and/or Glu which may improve solubility. Any flexible linker known and reported in the art may be used.

The use of GS linkers, or more particularly GS (“Gly-Ser”) domains in linkers, may allow the length of the linker readily to be varied by varying the number of GS domain repeats, and so such linkers represent one suitable class of linkers. However, flexible linkers are not limited to those based on “GS” repeats, and other linkers comprising Ser and Gly residues dispersed throughout the linker sequence have been reported, including in Chen et al., supra.

In one embodiment, the linker sequence comprises at least one Gly-Ser domain composed solely of Ser and Gly residues. In such an embodiment, the linker may contain no more than 15 other amino acid residues, e.g. no more than 14, 13, 12, 11, 10, 9, 8, 6, 7, 5, or 4 other amino acid residues.

The Gly-Ser domain may have the formula:

(S)q-[(G)m-(S)m]n-(G)p
wherein q is 0 or 1; m is an integer from 1-8; n is an integer of at least 1 (e.g. from 1 to 8, or more particularly 1 to 6); and p is 0 or an integer from 1 to 3.

More particularly, the Gly-Ser domain may have the formula:

S-[(G)m-S]n; (i)

[(G)m-S]n; or (ii)

[(G)m-S]n-(G)p (iii)
wherein m is an integer from 2-8 (for example 3-4); n is an integer of at least 1 (for example from 1 to 8, or more particularly 1 to 6); and p is 0 or an integer from 1 to 3.

In a representative example, the Gly-Ser domain may have the formula:

S-[G-G-G-G-S]n
wherein n is an integer of at least one (preferably 1 to 8, or 1-6, 1-5, 1-4, or 1-3) (where [G-G-G-G-S] is SEQ ID NO: 156).

The use of GS linkers, or more particularly GS (“Gly-Ser”) domains in linkers, may allow the length of the linker readily to be varied by varying the number of GS domain repeats, and so such linkers represent an advantageous type of linker to use. However, flexible linkers are not limited to those based on “GS” repeats, and other linkers comprising Ser and Gly residues dispersed throughout the linker sequence have been reported, including in Chen et al., supra.

A linker sequence may be composed solely of, or may consist of, one or more Gly-Ser domains as described or defined above. However, as noted above, the linker sequence may comprise one or more Gly-Ser domains, and additional amino acids. The additional amino acids may be at one or both ends of a Gly-Ser domain, or at one or both ends of a stretch of repeating Gly-Ser domains. Thus, the additional amino acid, which may be other amino acids, may lie at one or both ends of the linker sequence, e.g. they may flank the Gly-Ser domain(s). In other embodiments, the additional amino acids may lie between Gly-Ser domains. For example, two Gly-Ser domains may flank a stretch of other amino acids in the linker sequence. Further, as also noted above, in other linkers, GS domains need not be repeated, and G and/or S residues, or a short domain such as GS, may simply be distributed along the length or the sequence.

Representative exemplary linker sequences are listed below:

(SEQ ID NO: 161)
ETSGGGGSRL
(SEQ ID NO: 162)
SGGGGSGGGGSGGGGS
(where GGGGS is SEQ ID NO: 156)
S(GGGGS)₁₋₅
(where GGGGS is SEQ ID NO: 156)
(GGGGS)₁₋₅
(where GGGS is SEQ ID NO: 163)
S(GGGS)₁₋₅
(where GGGS is SEQ ID NO: 163)
(GGGS)₁₋₅
(where GGGGGS is SEQ ID NO: 164)
S(GGGGGS)₁₋₅
(where GGGGGS is SEQ ID NO: 164)
(GGGGGS)₁₋₅
(where GGGGGGS is SEQ ID NO: 165)
S(GGGGGGS)₁₋₅
(where GGGGGGS is SEQ ID NO: 165)
(GGGGGGS)₁₋₅
(SEQ ID NO: 159)
G₆
(SEQ ID NO: 160)
G₈
(SEQ ID NO: 166)
KESGSVSSEQLAQFRSLD
(SEQ ID NO: 167)
EGKSSGSGSESKST
(SEQ ID NO: 168)
GSAGSAAGSGEF
(SEQ ID NO: 169)
SGGGGSAGSAAGSGEF
(SEQ ID NO: 170)
SGGGLLLLLLLLGGGS
(SEQ ID NO: 171)
SGGGAAAAAAAAGGGS
(SEQ ID NO: 172)
SGGGAAAAAAAAAAAAAAAAGGGS
(SEQ ID NO: 173)
SGALGGLALAGLLLAGLGLGAAGS
(SEQ ID NO: 174)
SLSLSPGGGGGPAR
(SEQ ID NO: 175)
SLSLSPGGGGGPARSLSLSPGGGGG
(SEQ ID NO: 176)
GSSGSS
(SEQ ID NO: 177)
GSSSSSS
(SEQ ID NO: 178)
GGSSSS
(SEQ ID NO: 179)
GSSSSS
(SEQ ID NO: 180)
SGGGGS.

The linker or hinge may comprise or consist of at least 2, 3, 4, 5, 6, 7, 8, 9, 10, 11, 12, 13, 14, 15, 16, 17, 18, 19, 20, 25 or 30 amino acids, e.g. at least 2, 3, 4, 5, 6, 7, 8, 9 or 10 glycine residues. In a most particular embodiment, the chimeric receptor (e.g., CAR) described herein comprises an endodomain comprising a first amino acid sequence derived from IL2Rγ comprising a JAK3 binding domain (e.g. SEQ ID NOs 25 or 26) and a second amino acid sequence derived from IL2Rβ comprising a STAT5 association motif and a JAK1 and/or JAK2 binding motif (e.g. SEQ ID NOs 23 or 24), where the first and second amino acid sequences are connected or joined by a linker or hinge. For example, the chimeric receptor (e.g., CAR) may comprise an endodomain comprising SEQ ID NO: 25 and SEQ ID NO: 23 wherein SEQ ID NOs 25 and 23 are connected by a linker or hinge. A representative chimeric receptor (e.g., CAR) endodomain sequence may comprise or consist of the sequence shown in SEQ ID NO: 54, or a functional variant thereof.

Linkers which may be used in a chimeric receptor encoded by a nucleic acid of the invention include:

(SEQ ID NO: 155)
GGGGSGGGGSGGGGS
(SEQ ID NO: 156)
GGGGS
(SEQ ID NO: 157)
GGGGSGGGGS
(SEQ ID NO: 158)
GGGGSGGGGSGGGGSGGGGS
(SEQ ID NO: 159)
GGGGGG
(SEQ ID NO: 160)
GGGGGGGG

Other linkers which may be used include:

(SEQ ID NO: 166)
KESGSVSSEQLAQFRSLD
(SEQ ID NO: 167)
EGKSSGSGSESKST
(SEQ ID NO: 168)
GSAGSAAGSGEF

The exemplary constructs as shown in FIG. 15 comprise the following linkers:

Construct 4 (SEQ ID NO: 186): SEQ ID NO: 155 linker
Construct 5 (SEQ ID NO: 187): SEQ ID NO: 158 linker
Construct 6 (SEQ ID NO: 188): SEQ ID NO: 159 linker
Construct 7 (SEQ ID NO: 189): SEQ ID NO: 160 linker
Construct 8 (SEQ ID NO: 190): SEQ ID NO: 166 linker
Construct 9 (SEQ ID NO: 191): SEQ ID NO: 167 linker
Construct 10 (SEQ ID NO: 192): SEQ ID NO: 168 linker

Any of these linkers may be used in the context of other, similar, constructs, with differing endodomain STAT5 and JAK1/2, and JAK3 motif sequences. These linkers may for example be used in constructs comprising: the JAK3 binding motif sequence of SEQ ID NO. 25 or a variant thereof—any aforesaid linker sequence—the STAT5 association motif/JAK1 and/or JAK2 binding motif sequence of SEQ ID NO: 23 or a variant thereof.

Thus, particularly, a chimeric receptor (e.g., CAR) encoded by a nucleic acid of the invention may comprise an endodomain comprising the following domains/linkers:

JAK3 binding motif-SEQ ID NO: 155 linker-STAT5 association motif/JAK1 and/or JAK2 binding motif

JAK3 binding motif-SEQ ID NO: 158 linker-STAT5 association motif/JAK1 and/or JAK2 binding motif

JAK3 binding motif-SEQ ID NO: 159 linker-STAT5 association motif/JAK1 and/or JAK2 binding motif.

More particularly, a chimeric receptor (e.g., CAR) encoded by a nucleic acid of the invention may comprise an endodomain comprising the following domains/linkers:

### SEQ ID NO: 25-SEQ ID NO: 155-SEQ ID NO: 23

### SEQ ID NO: 25-SEQ ID NO: 158-SEQ ID NO: 23

### SEQ ID NO: 25-SEQ ID NO: 159-SEQ ID NO: 23

### SEQ ID NO: 26-SEQ ID NO: 155-SEQ ID NO: 23

### SEQ ID NO: 26-SEQ ID NO: 158-SEQ ID NO: 23

### SEQ ID NO: 26-SEQ ID NO: 159-SEQ ID NO: 23

### SEQ ID NO: 25-SEQ ID NO: 155-SEQ ID NO: 24

### SEQ ID NO: 25-SEQ ID NO: 158-SEQ ID NO: 24

### SEQ ID NO: 25-SEQ ID NO: 159-SEQ ID NO: 24

### SEQ ID NO: 26-SEQ ID NO: 155-SEQ ID NO: 24

### SEQ ID NO: 26-SEQ ID NO: 158-SEQ ID NO: 24

### SEQ ID NO: 26-SEQ ID NO: 159-SEQ ID NO: 24

Thus, as described above, the present invention thus provides a nucleic acid molecule comprising a polynucleotide sequence encoding a chimeric receptor (e.g., chimeric antigen receptor (CAR)) wherein said chimeric receptor (e.g. CAR) comprises an endodomain comprising (i) a STAT5 association motif, and a JAK1 and/or JAK2 binding motif and (ii) a JAK3 binding motif, wherein (i) and (ii) are connected by a linker or hinge. A vector comprising the nucleic acid molecule is further provided for, e.g. a lentiviral or retroviral vector.

A chimeric receptor (e.g., CAR) comprising an endodomain comprising (i) a STAT5 association motif, and a JAK1 and/or JAK2 binding motif and (ii) a JAK3 binding motif, wherein (i) and (ii) are connected by a linker or hinge, is also provided for by the invention. As described herein (i) maybe derived from IL2R13 e.g. SEQ ID NO: 23 or 24 and (ii) may be derived from IL2Ry (e.g. SEQ ID NO: 25 or 26).

The invention also provides a cell e.g. a T cell (particularly a Treg cell) or a NK (natural killer) cell comprising: a polynucleotide sequence encoding a chimeric receptor (e.g., chimeric antigen receptor (CAR)) wherein said chimeric receptor (e.g., CAR) comprises an endodomain comprising (i) a STAT5 association motif, and a JAK1 and/or JAK2 binding motif and (ii) a JAK3 binding motif, wherein (i) and (ii) are connected by a linker or hinge; a vector comprising said polynucleotide sequence; and/or a chimeric receptor (e.g., CAR) comprising an endodomain comprising (i) a STAT5 association motif, and a JAK1 and/or JAK2 binding motif and (ii) a JAK3 binding motif, wherein (i) and (ii) are connected by a linker or hinge, and a use of the cell e.g. T cell or NK cell in therapy, particularly for the therapeutic uses described herein. Pharmaceutical compositions comprising said cell are also provided by the invention. It will be appreciated that NK and Tconv cells and pharmaceutical compositions comprising said cells will also be capable of treating cancer, e.g. lung, liver, breast, prostate, ovarian, pancreatic cancer and blood cancers, such as leukaemia and lymphoma.

A method for producing a cell of the invention is further provided, comprising introducing to a cell, a polynucleotide sequence encoding a chimeric receptor (e.g., chimeric antigen receptor (CAR)) wherein said chimeric receptor (e.g. CAR) comprises an endodomain comprising (i) a STAT5 association motif, and a JAK1 and/or JAK2 binding motif and (ii) a JAK3 binding motif, wherein (i) and (ii) are connected by a linker or hinge. Cells obtainable by the method are encompassed by the invention.

The endodomain of a chimeric receptor (e.g., CAR) as described herein may also comprise motifs necessary to transduce an effector function signal and direct the cell (e.g., Treg) to perform its specialized function upon antigen binding. Examples of intracellular signaling domains include, but are not limited to, ζ chain endodomain of the T-cell receptor or any of its homologs (e.g., η chain, FcεR1γ and β chains, MB1 (Igα) chain, B29 (Igβ) chain, etc.), CD3 polypeptide domains (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human CD3 zeta chain endodomain, FcγRIII, FcsRI, cytoplasmic tails of Fc receptors, immunoreceptor tyrosine-based activation motif (ITAM) bearing cytoplasmic receptors or combinations thereof.

Preferably, the intracellular signaling domain comprises the intracellular signaling domain of a human CD3 zeta chain.

In one embodiment the intracellular signaling domain of human CD3 zeta chain comprises or consists of the following sequence:

UNIPROT: P20963, CD3Z_HUMAN, position 31-143
(SEQ ID NO: 27)
RVKFSRSADAPAYQQGQNQLYNELNLGRREEYDVLDKRRGRDPEMG
GKPQRRKNPQEGLYNELQKDKMAEAYSEIGMKGERRRGKGHDGLYQ
GLSTATKDTYDALHMQALPPR

In one embodiment, the intracellular signaling domain comprises at least 85, 90, 95, 97, 98 or 99% identity to SEQ ID NO: 27.

The intracellular signaling domain of the chimeric receptor (e.g., CAR) may comprise the following CD28 signaling domain:

(SEQ ID NO: 28)
RSKRSRLLHSDYMNMTPRRPGPTRKHYQPYAPPRDFAAYRS

In one embodiment, the intracellular signaling domain a signaling motif which has at least 85, 90, 95, 97, 98 or 99% identity to SEQ ID NO: 28.

The intracellular signaling domain of the chimeric receptor (e.g., CAR) may comprise the following CD27 signaling domain:

(SEQ ID NO: 29)
QRRKYRSNKGESPVEPAEPCHYSCPREEEGSTIPIQEDYRKPEPACSP.

In one embodiment, the intracellular signaling domain a signaling motif which has at least 85, 90, 95, 97, 98 or 99% identity to SEQ ID NO: 29.

Additional intracellular signaling domains will be apparent to those of skill in the art and may be used in connection with alternate embodiments of the invention.

The present chimeric receptor (e.g., CAR) may comprise a compound endodomain comprising a fusion of the intracellular part of a T-cell co-stimulatory molecule to that of e.g. CD3. Such a compound endodomain may be referred to as a second generation chimeric receptor (e.g., CAR) which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. The chimeric receptor (e.g., CAR) endodomain may also comprise one or more TNF receptor family signalling domain, such as the signalling domain of OX40, 4-1BB, ICOS or TNFRSF25.

Illustrative sequences for OX40, 4-1BB, ICOS and TNFRSF25 signalling domains are shown below as SEQ ID NO: 30-33. The chimeric receptor (e.g., CAR) endodoman may also comprise one or more of SEQ ID NO: 30-33 or a variant of SEQ ID NO: 30-33.

OX40 signalling domain (SEQ ID NO: 30):
ALYLLRRDQRLPPDAHKPPGGGSFRTPIQEEQADAHSTLAKI
41BB signalling domain (SEQ ID NO: 31):
KRGRKKLLYIFKQPFMRPVQTTQEEDGCSCRFPEEEEGGCEL
ICOS signalling domain (SEQ ID NO: 32):
CWLTKKKYSSSVHDPNGEYMFMRAVNTAKKSRLTDVTL
TNFRSF25 signalling domain (SEQ ID NO: 33):
TYTYRHCWPHKPLVTADEAGMEALTPPPATHLSPLDSAHTLLAPPDSS
EKICTVQLVGNSWTPGYPETQEALCPQVTWSWDQLPSRALGPAAAPTL
SPESPAGSPAMMLQPGPQLYDVMDAVPARRWKEFVRTLGLREAEIEAV
EVEIGRFRDQQYEMLKRWRQQQPAGLGAVYAALERMGLDGCVEDLRSR
LQRGP

The endodomain may comprise further sequences, for example parts of the CD8 and/or CD28 endodomains, in addition to the co-stimulatory and signalling domains indicated above. Additional sequences may be included to facilitate cloning of the chimeric receptor sequence, for example to add or remove restriction cleavage sites from the coding sequence, and to facilitate the construction of the chimeric receptor or its coding sequence.

The chimeric receptor (e.g., CAR) endodomain may comprise a variant of one or more of SEQ ID NO: 30-33 which has at least 85, 90, 95, 97, 98 or 99% identity to any one of SEQ ID NO: 30-33.

Suitably, the chimeric receptor (e.g., CAR) endodomain may comprise or consist of SEQ ID NO: 45 or a variant which has at least 85, 90, 95, 97, 98 or 99% identity to SEQ ID NO: 45.

(illustrative endodomain sequence comprising CD28, IL2RG-T52, IL2RB-
Y510, CD3 zeta signalling domains)
SEQ ID NO: 45
RSKRSRLLHSDYMNMTPRRPGPTRKHYQPYAPPRDFAAYRSERTMPRIPTLKNLEDLVTEYHGNFSAW
SGVSKGLAESLQPDYSERLCLVSEINCRNTGPWLKKVLKCNTPDPSKFFSQLSSEHGGDVQKWLSSPF
PSSSFSPGGLAPEISPLEVLERDKVTQLLPLNTDAYLSLQELQGQDPTHLVRVKFSRSADAPAYQQGQ
NQLYNELNLGRREEYDVLDKRRGRDPEMGGKPRRKNPQEGLYNELQKDKMAEAYSEIGMKGERRRGKG
HDGLYQGLSTATKDTYDALHMQALPPR

Suitably, the chimeric receptor (e.g., CAR) endodomain may comprise or consist of SEQ ID NO: 53 or a variant which has at least 85, 90, 95, 97, 98 or 99% identity to SEQ ID NO: 53.

(illustrative endodomain sequence comprising CD28, IL2RG-T52, IL7RA-2Y,
CD3 zeta signalling domains)
SEQ ID NO: 53
RSKRSRLLHSDYMNMTPRRPGPTRKHYQPYAPPRDFAAYRSERTMPRIPTLKNLEDLVTEYHGNFSAW
SGVSKGLAESLQPDYSERLCLVSEIKKRIKPIVWPSLPDHKKTLEHLCKKPRKNLNVSFNPESFLDCQ
IHRVDDIQARDEVEGFLQDTFPQQPILTSLGSNQEEAYVTMSSFYQNQRVKFSRSADAPAYQQGQNQL
YNELNLGRREEYDVLDKRRGRDPEMGGKPRRKNPQEGLYNELQKDKMAEAYSEIGMKGERRRGKGHDG
LYQGLSTATKDTYDALHMQALPPRGSGATNFSLLKQAGDVEENPG

Suitably, the chimeric receptor (e.g., CAR) endodomain may comprise or consist of SEQ ID NO:154 or a variant which has at least 85, 90, 95, 97, 98 or 99% identity to SEQ ID NO: 154.

(illustrative endodomain sequence comprising CD28, IL2RG-T52, linker,
IL2RB-Y510, CD3 zeta signalling domains)
SEQ ID NO: 154
RSKRSRLLHSDYMNMTPRRPGPTRKHYQPYAPPRDFAAYRS [symbol] [symbol] [symbol]
GGGGSGGGGSGGGG [symbol] [symbol] [symbol] [symbol] [symbol]
RVKFS
RSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR

Other illustrative endodomains include endodomains comprising a CD28 intracellular domain (including or consisting of the CD28 costimulatory domain), the JAK3 binding motif domain IL2RG-T52 sequence of SEQ ID NO: 25, a linker sequence, the STAT5 association motif/JAK1 and/or JAK2 binding motif domain sequence of SEQ ID NO.23 and the CD3 zeta signalling domain. Such endodomain sequences are contained for example in the constructs CIV-CX as depicted in FIG. 15 and described in Example 10 below.

The endodomain of any of the constructs CIV-CX of FIG. 15/Example 10 may be used on the context of a chimeric receptor according to any aspect of the invention herein. Accordingly, the endodomain may comprise or consist of the endodomain sequence of any one of constructs CIV to CX as shown in SEQ ID NOS: 198 to 204 respectively, or a variant of any one of SEQ ID NOS: 198 to 204 which has at least 85, 90, 95, 97, 98 or 99% identity thereto. These endodomains and possible modifications thereof are discussed further in Example 10 below.

Any such endodomain may be combined in the chimeric receptor with a CD8 TM and/or hinge domain, for example as CD8 TM and/or hinge domain as indicated above.

### Variants, Derivatives and Fragments

In addition to the specific proteins, peptides and nucleotides mentioned herein, the present invention also encompasses the use of derivatives and fragments thereof.

The term “derivative” or “variant” as used interchangeably herein, in relation to proteins or polypeptides of the present invention includes any substitution of, variation of, modification of, replacement of, deletion of and/or addition of one (or more) amino acid residues from or to the sequence providing that the resultant protein or polypeptide retains the desired function (for example, where the derivative or variant is an antigen binding domain, the desired function may be the ability of the antigen binding domain to bind its target antigen, or where the derivative or variant is a signalling domain, the desired function may be the ability of that domain to signal (e.g. activate or inactivate a downstream molecule). For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding, reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The derivative may be a homolog. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence.

Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally. “Fragment” thus refers to an amino acid or nucleic acid sequence that is a portion of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Transmembrane Domain

The chimeric receptor (e.g., CAR) may also comprise a transmembrane domain. The transmembrane domain may comprise the transmembrane sequence from any protein which has a transmembrane domain, including any of the type I, type II or type III transmembrane proteins. The transmembrane domain of the chimeric receptor (e.g., CAR) may also comprise an artificial hydrophobic sequence. The transmembrane domains of the chimeric receptor (e.g., CAR) may be selected so as not to dimerize. Additional transmembrane domains will be apparent to those of skill in the art. Examples of transmembrane (TM) regions used in chimeric receptor (e.g., CAR) constructs are: 1) The CD28 TM region (Pule et al, Mol Ther, 2005, November; 12(5):933-41; Brentjens et al, CCR, 2007, Sep. 15; 13 (18 Pt 1):5426-35; Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13 (18 Pt 1):5426-35); 4) The CD3 zeta TM region (Pule et al, Mol Ther, 2005, Nov.; 12(5):933-41; Savoldo B, Blood, 2009, Jun. 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, Sep. 15; 13 (18 Pt 1):5426-35; Milone et al, Mol Ther, 2009, August; 17(8):1453-64.).

In an alternative embodiment, the transmembrane domain may be selected to comprise a dimerisation domain, allowing dimerisation of the chimeric receptor, where desirable. Exemplary dimerisation domains are disclosed in WO2019/169290, incorporated herein by reference.

Suitably, the transmembrane domain may comprise the amino acid sequence shown as SEQ ID NO: 35, or a variant which is at least 80% identical to SEQ ID NO: 35

CD28 Transmembrane
SEQ ID NO: 35
FWVLVVVGGVLACYSLLVTVAFIIFWV

Suitably, the variant may be at least 85, 90, 95, 97, 98 or 99% identical to SEQ ID NO: 35.

Suitably, the chimeric receptor (e.g., CAR) may comprise the CD8a transmembrane domain. Suitably, the transmembrane domain may comprise the amino acid sequence shown as SEQ ID NO: 87, or a variant which is at least 80% identical to SEQ ID NO: 87.

Illustrative CD8α TM domain (AA 183 to 203)
(SEQ ID NO: 87)
IYIWAPLAGTCGVLLLSLVIT

An alternative CD8 TM domain sequence is shown in SEQ ID NO: 195.

(SEQ ID NO: 195)
IYTWAPLAGTCGVLLLSLVITLYC

Suitably, the transmembrane domain may comprise the amino acid sequence shown as SEQ ID NO: 195, or a variant which is at least 80% identical to SEQ ID NO: 195

Suitably, the variant may be at least 85, 90, 95, 97, 98 or 99% identical to SEQ ID NO: 87 or SEQ ID NO: 195.

Suitably, the chimeric receptor (e.g., CAR) may comprise the CD28 hinge and transmembrane sequence. Suitably, the hinge and transmembrane domain may comprise the amino acid sequence shown as SEQ ID NO: 36, or a variant which is at least 80% identical to SEQ ID NO: 36

CD28 transmembrane
SEQ ID NO: 36
IEVMYPPPYLDNEKSNGTIIHVKGKHLCPSPLFPGPSKPFWVLVVVGGV
LACYSLLVTVAFIIFWV

Suitably, the variant may be at least 85, 90, 95, 97, 98 or 99% identical to SEQ ID NO: 36.

In one embodiment the transmembrane and intracellular signaling domain are both derived from CD28. In one embodiment the transmembrane and intracellular signaling domain comprise the sequence below:

Transmembrane and intracellular portion of the
human CD28 (UNIPROT: P10747, CD28_HUMAN, position
153-220)
(SEQ ID NO: 37)
FWVLVVVGGVLACYSLLVTVAFIIFWVRSKRSRLLHSDYMNMTPRRPGP
TRKHYQPYAPPRDFAAYRS

In one embodiment the transmembrane and intracellular signaling domain comprises at least 85, 90, 95, 97, 98 or 99% identity to SEQ ID NO: 37.

In one embodiment the transmembrane domain of CD28 comprises the sequence FWVLVVVGGVLACYSLLVTVAFIIFWV (SEQ ID NO: 38).

As discussed previously, the transmembrane domain may comprise a portion or all of the transmembrane region from IL2R13 or IL2Rgamma.

Suitably, the chimeric receptor (e.g., CAR) may encode a tag—such as a c-Myc tag (EQKLISEEDL—SEQ ID NO: 39). Suitably the tag may be incorporated into the extracellular domain of the chimeric receptor (e.g., CAR), for example in the hinge region of the extracellular domain. An illustrative CD28 hinge/transmembrane domain with an integrated c-Myc tag is shown as SEQ ID NO: 40.

(SEQ ID NO: 40)
IEVEQKLISEEDLLDNEKSNGTIIHVKGKHLCPSPLFPGPSKPFWVLVV
VGGVLACYSLLVTVAFIIEWV.

Suitably, the chimeric receptor (e.g., CAR) may comprise the CD8a hinge domain, and/or the CD8a transmembrane domain. Further, the chimeric receptor (e.g., CAR) may comprise the CD8a hinge domain and the CD28 transmembrane domain. Suitably, the hinge and transmembrane domain may comprise the amino acid sequence shown as SEQ ID NO: 88, or a variant which is at least 80% identical to SEQ ID NO: 88.

Illustrative CD8α hinge domain and the CD28
transmembrane domain (SEQ ID NO: 88):
TTTPAPRPPTPAPTIASQPLSLRPEACRPAAGGAVHTRGLDFACDFWVL
VVVGGVLACYSLLVTVAFIIFWV

Suitably, the variant may be at least 85, 90, 95, 97, 98 or 99% identical to SEQ ID NO: 88.

CD8α hinge-
(SEQ ID NO: 181)
TTTPAPRPPTPAPTIASQPLSLRPEACRPAAGGAVHTRGLDFACD
CD8α hinge (also referred to as CD8 extracellular
domain)
(SEQ ID NO: 194)
FVPVFLPAKPTTTPAPRPPTPAPTIASQPLSLRPEACRPAAGGAVHTRG
LDFACD

In some embodiments the chimeric receptor (e.g. CAR) may comprise a CD8, particularly a CD8a, hinge sequence. The hinge domain may comprise the amino acid sequence as shown in SEQ ID NO: 181 or 194, or a variant which is at least 80% identical to SEQ ID NO: 181 or 194. Suitably, the variant may be at least 85, 90, 95, 97, 98 or 99% identical to SEQ ID NO: 181 or 194.

Suitably, the chimeric receptor (e.g., CAR) may comprise the CD28 hinge domain and the CD8a transmembrane domain. Suitably, the hinge and transmembrane domain may comprise the amino acid sequence shown as SEQ ID NO: 89, or a variant which is at least 80% identical to SEQ ID NO: 89.

Illustrative CD28 hinge domain and the CD8α
transmembrane domain (SEQ ID NO: 89):
IEVMYPPPYLDNEKSNGTIIHVKGKHLCPSPLFPGPSKPIYIWAPLAGT
CGVLLLSLVIT

Suitably, the variant may be at least 85, 90, 95, 97, 98 or 99% identical to SEQ ID NO: 89.

The chimeric receptor (e.g., CAR) may further comprise a leader sequence which targets it to the endoplasmic reticulum pathway for expression on the cell surface. An illustrative leader sequence is MALPVTALLLPLALLLHAARP (SEQ ID NO: 41). This is the human CD8 leader.

Illustrative CARs for use in the present invention are shown as SEQ ID NO: 42-44.

(CAR containing HLA-A2 scFv, c-Myc tag, CD28, IL2RB-Y510, CD3 zeta
endodomain)
SEQ ID NO: 42
MALPVTALLLPLALLLHAARPQVQLVQSGGGVVQPGGSLRVSCAASGVTLSDYGMHWVRQAPGKGLEW
MAFIRNDGSDKYYADSVKGRFTISRDNSKKTVSLQMSSLRAEDTAVYYCAKNGESGPLDYWYFDLWGR
GTLVTVSSGGGGSGGGGSGGGGSDVVMTQSPSSLSASVGDRVTITCQASQDISNYLNWYQQKPGKAPK
LLIYDASNLETGVPSRFSGSGSGTDFTFTISSLQPEDIATYYCQQYDNLPPTFGGGTKLTVLGAAAIE
VEQKLISEEDLLDNEKSNGTIIHVKGKHLCPSPLFPGPSKPFWVLVVVGGVLACYSLLVTVAFIIFWV
RSKRSRLLHSDYMNMTPRRPGPTRKHYQPYAPPRDFAAYRSNCRNTGPWLKKVLKCNTPDPSKFFSQL
SSEHGGDVQKWLSSPFPSSSFSPGGLAPEISPLEVLERDKVTQLLPLNTDAYLSLQELQGQDPTHLVR
VKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPRETRGGGATMVSKGEELFTGVVPILV
ELDGDVNGHKFSVSGEGEGDATYGKLTLKEICTTGKLPVPWPTLVTTLTYGVQCFSRYPDHMKQHDEE
KSAMPEGYVQERTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSKLSKDPNEKRDHMVL
LEFVTAAGITLGMDELYK
(CAR containing HLA-A2 scFv, c-Myc tag, CD28, IL2RG-T52, IL2RB-Y5 10,
CD3 zeta endodomain)
SEQ ID NO: 43
MALPVTALLLPLALLLHAARPQVQLVQSGGGVVQPGGSLRVSCAASGVTLSDYGMHWVRQAPGKGLEW
MAFIRNDGSDKYYADSVKGRFTISRDNSKKTVSLQMSSLRAEDTAVYYCAKNGESGPLDYWYFDLWGR
GTLVTVSSGGGGSGGGGSGGGGSDVVMTQSPSSLSASVGDRVTITCQASQDISNYLNWYQQKPGKAPK
LLIYDASNLETGVPSRFSGSGSGTDFTFTISSLQPEDIATYYCQQYDNLPPTFGGGTKLTVLGAAAIE
VEQKLISEEDLLDNEKSNGTIIHVKGKHLCPSPLFPGPSKPFWVLVVVGGVLACYSLLVTVAFIIFWV
RSKRSRLLHSDYMNMTPRRPGPTRKHYQPYAPPRDFAAYRSERTMPRIPTLKNLEDLVTEYHGNFSAW
SGVSKGLAESLQPDYSERLCLVSEINCRNTGPWLKKVLKCNTPDPSKFFSQLSSEHGGDVQKWLSSPF
PSSSFSPGGLAPEISPLEVLERDKVTQLLPLNTDAYLSLQELQGQDPTHLVRVKFSRSADAPAYQQGQ
NQLYNELNLGRREEYDVLDKRRGRDPEMGGKPRRKNPQEGLYNELQKDKMAEAYSEIGMKGERRRGKG
HDGLYQGLSTATKDTYDALHMQALPPR
(CAR containing HLA-A2 scFv, c-Myc tag, CD28, IL2RG-T52, IL7RA-2Y,
CD3 zeta endodomain)
SEQ ID NO: 44
MALPVTALLLPLALLLHAARPQVQLVQSGGGVVQPGGSLRVSCAASGVTLSDYGMHWVRQAPGKGLEW
MAFIRNDGSDKYYADSVKGRFTISRDNSKKTVSLQMSSLRAEDTAVYYCAKNGESGPLDYWYFDLWGR
GTLVTVSSGGGGSGGGGSGGGGSDVVMTQSPSSLSASVGDRVTITCQASQDISNYLNWYQQKPGKAPK
LLIYDASNLETGVPSRFSGSGSGTDFTFTISSLQPEDIATYYCQQYDNLPPTFGGGTKLTVLGAAAIE
VEQKLISEEDLLDNEKSNGTIIHVKGKHLCPSPLFPGPSKPFWVLVVVGGVLACYSLLVTVAFIIFWV
RSKRSRLLHSDYMNMTPRRPGPTRKHYQPYAPPRDFAAYRSERTMPRIPTLKNLEDLVTEYHGNFSAW
SGVSKGLAESLQPDYSERLCLVSEIKKRIKPIVWPSLPDHKKTLEHLCKKPRKNLNVSFNPESFLDCQ
IHRVDDIQARDEVEGFLQDTFPQQPILTSLGSNQEEAYVTMSSFYQNQRVKFSRSADAPAYQQGQNQL
YNELNLGRREEYDVLDKRRGRDPEMGGKPRRKNPQEGLYNELQKDKMAEAYSEIGMKGERRRGKGHDG
LYQGLSTATKDTYDALHMQALPPRGSGATNFSLLKQAGDVEENPG

The CAR may comprise a sequence which is at least 85, 90, 95, 97, 98 or 99% identity to any one of SEQ ID NO: 42-44.

### Chimeric Antigen Receptor (CAR)

“Chimeric antigen receptor” or “CAR” or “CARs” as used herein refers to engineered receptors which can confer an antigen specificity onto cells (for example Tregs). CARs are also known as artificial T-cell receptors, chimeric T-cell receptors or chimeric immunoreceptors. Preferably the CARs as defined herein comprise an extracellular antigen-specific targeting region, a transmembrane domain, optionally one or more co-stimulatory domains, and an intracellular signaling domain (also referred to as an endodomain).

CAR-encoding polynucleotides may be transferred to a Treg using, for example, retroviral vectors. In this way, a large number of antigen-specific T cells can be generated for adoptive cell transfer. When the CAR binds the target-antigen, this results in the transmission of an activating signal to the Treg it is expressed on. Thus, the CAR directs the specificity of the engineered Treg towards cells expressing the targeted antigen.

### Antigen-Specific Targeting Domain

The antigen-specific targeting domain provides the CAR with the ability to bind a predetermined antigen of interest. The antigen-specific targeting domain preferably targets an antigen of clinical interest.

The antigen-specific targeting domain may be any protein or peptide that possesses the ability to specifically recognize and bind to a biological molecule (e.g., a cell surface receptor or a component thereof). The antigen-specific targeting domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/WIC or HLA combination which is capable of binding to the TCRs of Tcon cells active at a site of transplantation, inflammation or disease. Such antigen-binding domains have been reported for example in Mekala et al, Blood, 2005, vol 105, pages 2090-2092.

In a preferred embodiment, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) a camelid antibody (VHH) and a single domain antibody (sAb).

In a preferred embodiment, the binding domain is a single chain antibody (scFv). The scFv may be murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another directly or via a peptide linker sequence.

Antibodies that specifically bind a predetermined antigen can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

Antigens which may be targeted by the present CAR include, but are not limited to, antigens expressed on cells associated with transplanted organs, autoimmune diseases, allergic diseases and inflammatory diseases (e.g. neurodegenerative disease). It will be understood by a skilled person that due to the bystander effect of Treg cells, the antigen may be simply present and/or expressed at the site of transplantation, inflammation or disease.

Antigens expressed on cells associated with neurodegenerative disease include those presented on glial cells, e.g. MOG.

Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HNIlVIR, TOP2A, GPNMB, PLA2G7, CXCL9, FABP5, GBP2, CD74, CXCL10, UBD, CD27, CD48, CXCL11.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

The antigen recognition domain may bind, suitably specifically bind, one or more region or epitope within HLA-A2. An epitope, also known as antigenic determinant, is the part of an antigen that is recognised by an antigen recognition domain (e.g. an antibody). In other words, the epitope is the specific piece of the antigen to which an antibody binds. Suitably, the antigen recognition domain binds, suitably specifically binds, to one region or epitope within HLA-A2.

The antigen recognition domain may comprise at least one CDR (e.g. CDR3), which can be predicted from an antibody which binds to an antigen, preferably HLA-A2 (or a variant of such a predicted CDR (e.g. a variant with one, two or three amino acid substitutions)). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13: 1882-91).

In this respect, the antigen recognition domain may comprise one or more variable heavy chain CDRs, e.g. one, two or three variable heavy chain CDRs. Alternatively, or additionally, the antigen recognition domain may comprise one or more variable light chain CDRs, e.g. one, two or three variable light chain CDRs. The antigen recognition domain may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an antibody which binds to an antigen, preferably HLA-A2, or may be selected from one of the CDR sequences provided below.

The antigen recognition domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the antigen recognition domain comprises three variable heavy chain CDRs (CDR1, CDR2 and CDR3) or three variable light chain CDRs (CDR1, CDR2 and CDR3).

The one or more CDRs present within the antigen recognition domain may not all be from the same antibody, as long as the domain has the binding activity described above. Thus, one CDR may be predicted from the heavy or light chains of an antibody which binds to an antigen, e.g. HLA-A2 whilst another CDR present may be predicted from a different antibody which binds to the same antigen (e.g. HLA-A2). In this instance, it may be preferred that CDR3 be predicted from an antibody that binds to an antigen, e.g. HLA-A2. Particularly however, if more than one CDR is present in the antigen recognition domain, it is preferred that the CDRs are predicted from antibodies which bind to the same antigen, e.g. HLA-A2. A combination of CDRs may be used from different antibodies, particularly from antibodies that bind to the same desired region or epitope.

In a particularly preferred embodiment, the antigen recognition domain comprises three CDRs predicted from the variable heavy chain sequence of an antibody which binds to aa antigen, e.g. HLA-A2 and/or three CDRs predicted from the variable light chain sequence of an antibody which binds to an antigen, e.g. HLA-A2 (preferably the same antibody).

In some embodiments, the antigen recognition domain is, or is derived from an antibody (e.g. is a Fab, scFv, or sdAb) wherein the antibody comprises one or more CDR regions, selected from SEQ ID NOs: 90-146, or derivatives thereof. In other words, in some embodiments the antigen recognition domain comprises one or more CDR regions, selected from SEQ ID NOs: 90-146, or derivatives thereof. Suitably, the antigen recognition domain comprises three CDR regions selected from SEQ ID NOs: 90-146, or derivatives thereof.

| Name | CDR1 | CDR2 | CDR3 |
|---|---|---|---|
| GL VH CDRs | DYGMH | FIRNDGSDKYYADSVKG | NGESGPLDYWYFDL |
| (SEQ ID NOs: 90-92) | (SEQ ID NO: 90) | (SEQ ID NO: 91) | (SEQ ID NO: 92) |
| 3PB2 VH CDRs | DYGMH | FIRNDGSDKYYADSVKG | NGESGPLDYWYLDL |
| (SEQ ID NOs: 93-95) | (SEQ ID NO: 93) | (SEQ ID NO: 94) | (SEQ ID NO: 95) |
| 3PC4 VH CDRs | DYGMH | FIRNDGSDKYYADSVRG | NGESGPLDYWYFDL |
| (SEQ ID NOs: 96-98) | (SEQ ID NO: 96) | (SEQ ID NO: 97) | (SEQ ID NO: 98) |
| 3PF12 VH CDRs | DYGMH | FIRNDGSDKYYADSVKG | NGESGPLDYWYFDL |
| (SEQ ID NOs: 99-101) | (SEQ ID NO: 99) | (SEQ ID NO: 100) | (SEQ ID NO: 101) |
| GL VL CDRs | QASQDISNYLN | DASNLET | QQYDNLPPT |
| (SEQ ID NOs: 102-104) | (SEQ ID NO: 102) | (SEQ ID NO: 103) | (SEQ ID NO: 104) |
| 3PB2 VL CDRs | QSSLDISHYLN | DASNLET | QQYDNLPLT |
| (SEQ ID NOs: 105-107) | (SEQ ID NO: 105) | (SEQ ID NO: 106) | (SEQ ID NO: 107) |
| 3PC4 VL CDRs | RASHGINNYLA | AASTLQS | QQYDSYPPT |
| (SEQ ID NOs: 108-110) | (SEQ ID NO: 108) | (SEQ ID NO: 109) | (SEQ ID NO: 110) |
| 3PF12 VL CDRs | QASQDISNYLN | DASNLET | QQYSSFPLT |
| (SEQ ID NOs: 111-113) | (SEQ ID NO: 111) | (SEQ ID NO: 112) | (SEQ ID NO: 113) |
| C12 VL CDRs | QASQDISNYLN | DETHLDS | QQYDSLPPT |
| (SEQ ID NOs: 114-116) | (SEQ ID NO: 114) | (SEQ ID NO: 115) | (SEQ ID NO: 116) |
| E7 VL CDRs | QASQDISNYLN | DASNLET | QQYDNLPIT |
| (SEQ ID NOs: 117-119) | (SEQ ID NO: 117) | (SEQ ID NO: 118) | (SEQ ID NO: 119) |
| H10 VL CDRs | QASQDISNYLN | DASNLET | QQYDNLPST |
| (SEQ ID NOs: 120-122) | (SEQ ID NO: 120) | (SEQ ID NO: 121) | (SEQ ID NO: 122) |
| B8 VL CDRs | QASQDISNYLN | DASNLET | QQYNTYPLT |
| (SEQ ID NOs: 123-125) | (SEQ ID NO: 123) | (SEQ ID NO: 124) | (SEQ ID NO: 125) |
| D2 VL CDRs | QASQDISNYLN | DASNLET | QQYHTYPLT |
| (SEQ ID NOs: 126-128) | (SEQ ID NO: 126) | (SEQ ID NO: 127) | (SEQ ID NO: 128) |
| B10 VL CDRs | QASQDISNYLN | DASNLET | QQYDNLPLT |
| (SEQ ID NOs: 129-131) | (SEQ ID NO: 129) | (SEQ ID NO: 130) | (SEQ ID NO: 131) |
| 2A9 VL CDRs | RTSQGISSALA | DASSLES | QQFNNYPLT |
| (SEQ ID NOs: 132-134) | (SEQ ID NO: 132) | (SEQ ID NO: 133) | (SEQ ID NO: 134) |
| 3B12 VL CDRs | QASQDISNYLA | AASNLQS | LQDSSYPPT |
| (SEQ ID NOs: 135-137) | (SEQ ID NO: 135) | (SEQ ID NO: 136) | (SEQ ID NO: 137) |
| 2D4 VL CDRs | RASQSISSWLA | KASNLQS | QQYSNYPLT |
| (SEQ ID NOs: 138-140) | (SEQ ID NO: 138) | (SEQ ID NO: 139) | (SEQ ID NO: 140) |
| 3D4 VL CDRs | RASHGISNYFA | ATSTLQS | QQYSSYPLT |
| (SEQ ID NOs: 141-143) | (SEQ ID NO: 141) | (SEQ ID NO: 142) | (SEQ ID NO: 143) |
| B3 VL CDRs | RASRGSNYLA | ATSTLQS | QQYDSYPPT |
| (SEQ ID NOs: 144-146) | (SEQ ID NO: 144) | (SEQ ID NO: 145) | (SEQ ID NO: 146) |

Preferably, the antigen binding domain comprises CDRs (CDR1, CDR2, and CDR3), or derivatives thereof, selected from the same variable chain. For example, the antigen binding domain may comprise SEQ ID NOs: 90-92, SEQ ID NOs: 93-95, SEQ ID NOs: 96-98, SEQ ID NOs: 99-101, SEQ ID NOs: 102-104, SEQ ID NOs: 105-107, SEQ ID NOs: 108-110, SEQ ID NOs: 111-113, SEQ ID NOs: 114-116, SEQ ID NOs: 117-119, SEQ ID NOs: 120-122, SEQ ID NOs: 123-125, SEQ ID NOs: 126-128, SEQ ID NOs: 129-131, SEQ ID NOs: 132-134, SEQ ID NOs: 135-137, SEQ ID NOs: 138-140, SEQ ID NOs: 141-143, and/or SEQ ID NOs: 144-146, or derivatives thereof.

In preferred embodiments, the antigen recognition domain comprises a combination variable heavy and variable light CDRs as follows:
(i) SEQ ID NOs: 90-92 and SEQ ID NOs: 102-104, or derivatives thereof;
(ii) SEQ ID NOs: 93-95 and SEQ ID NOs: 105-107, or derivatives thereof;
(iii) SEQ ID NOs: 96-98 and SEQ ID NOs: 108-110, or derivatives thereof;
(iv) SEQ ID NOs: 99-101 and SEQ ID NOs: 111-113, or derivatives thereof;
(v) SEQ ID NOs: 99-101 and SEQ ID NOs: 114-116, or derivatives thereof;
(vi) SEQ ID NOs: 99-101 and SEQ ID NOs: 117-119, or derivatives thereof;
(vii) SEQ ID NOs: 99-101 and SEQ ID NOs: 120-122, or derivatives thereof;
(viii) SEQ ID NOs: 99-101 and SEQ ID NOs: 123-125, or derivatives thereof;
(ix) SEQ ID NOs: 99-101 and SEQ ID NOs: 126-128, or derivatives thereof;
(x) SEQ ID NOs: 99-101 and SEQ ID NOs: 129-131, or derivatives thereof;
(xi) SEQ ID NOs: 99-101 and SEQ ID NOs: 132-134, or derivatives thereof;
(xii) SEQ ID NOs: 99-101 and SEQ ID NOs: 135-137, or derivatives thereof;
(xiii) SEQ ID NOs: 99-101 and SEQ ID NOs: 138-140, or derivatives thereof;
(xiv) SEQ ID NOs: 99-101 and SEQ ID NOs: 141-143, or derivatives thereof;
(xv) SEQ ID NOs: 99-101 and SEQ ID NOs: 144-146, or derivatives thereof;

Preferably, the antigen recognition domain comprises SEQ ID NOs: 93-95 and SEQ ID NOs: 105-107, or derivatives thereof.

The antigen binding domain may comprise a variable heavy domain selected from SEQ ID NO: 54, 55, 56 or 57 or a variant which is at least 80% identical to SEQ ID NO: 54, 55, 56 or 57. The variant which may be at least 85, 90, 95, 97, 98 or 99% identical to SEQ ID NO: 54, 55, 56 or 57.

SEQ ID NO: 54
VQLVQSGGGVVQPGGSLRVSCAASGVTLSDYGMHWVRQAPGKGLEWMAF
IRNDGSDKYYADSVKGRFTISRDNSKKTVSLOMSSLRAEDTAVYYCAKN
GESGPLDYWYFDLWGRGTLVTV
SEQ ID NO: 55
QVQLVOSGGGVVQPGGSLRVSCAASGVTLSDYGMHWVRQAPGKGLEWVA
FIRNDGSDKYYADSVKGRFTISRDNSEKTVSLQMSSLRAEDTAVYYCAK
NGESGPLDYWYLDLWGRGT
SEQ ID NO: 56
QVQLVOSGGGVVQPGGSMRVSCAASGVTLSDYGMHWVRQAPGKGLEWVA
FIRNDGSDKYYADSVRGRFTISRDNSKKTVFLQMNSLRAEDTAVYYCAK
NGESGPLDYWYFDLWGRGT
SEQ ID NO: 57
QVQLVOSGGGVVQPGGSLRVSCAASGVTLSDYGMHWVRQAPGKGLEWMA
FIRNDGSDKYYADSVKGRFTISRDNSKKTVSLQMSSLRAEDTAVYYCAK
NGESGPLDYWYFDLWGRGT

The antigen binding domain may comprise a variable light domain selected from SEQ ID NO: 58 to 72 or a variant which is at least 80% identical to SEQ ID NO: 58 to 72. The variant which may be at least 85, 90, 95, 97, 98 or 99% identical to SEQ ID NO: 58 to 72.

SEQ ID NO: 58
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPPTFGG
GTKLTVLG
SEQ ID NO: 59
DVVMTQSPSSLSASVGDRVTITCQSSLDISHYLNWYQQKPGKAPKLLIYD
ASNLETGVPSRFSGSGSGTHFTFTISSLQPEDFATYYCQQYDNLPLTFGG
GTKLEIK
SEQ ID NO: 60
DIVLMQSPSFLSASVGDRVTITCRASHGINNYLAWYQQKPGKAPKLLIYA
ASTLQSGVPSRFSGSGSGTEFTLTISSLQPEDFATYYCQQYDSYPPTFGR
TKVEIKR
SEQ ID NO: 61
DVVMTQSPSSLSASVGDRVTITCQASQDISNYLNWYQQKPGKAPKLLIYD
ASNLETGVPSRFSGSGSGTDFTFTISSLQPEDFATYYCQQYSSFPLTFGG
GTKVDIK
SEQ ID NO: 62
DVVMTQSPSSLSASVGDRVTITCQASQDISNYLNWYQQEPGKAPKLLIYD
ETHLDSGVPSRFTGSRSGTDFTLTISSLQPEDFATYYCQQYDSLPPTFGG
GTKVDIK
SEQ ID NO: 63
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPITFGG
GTKVDIK
SEQ ID NO: 64
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPSTFGG
GTKVDIK
SEQ ID NO: 65
DVVMTQSPSSLSASVGDRVTITCQASQDISNYLNWYQQKPGKAPKLLIYD
ASNLETGVPSRFSGSGSGTDFTFTISSLQPEDFGTYYCQQYNTYPLTFGG
GTKVDIK
SEQ ID NO: 66
DVVMTQSPSSLTASVGDRVTITCQASQDISNYLNWYQQKPGKAPKLLIYD
ASNLETGVPSRFSGSGSGTDFTLSIDSLQPEDFATYYCQQYHTYPLTFGG
GTKVDIK
SEQ ID NO: 67
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPLTFGG
GTKVDIK
SEQ ID NO: 68
DVVMTQSPSSLSASVGDRVTITCRTSQGISSALAWYQQKPGKAPKLLIYD
ASSLESGVPSRFSGSGSGTDFTLTISSLQPEDFATYYCQQFNNYPLTFGG
GTKVDIK
SEQ ID NO: 69
DVVMTQSPSSLSASVGDRVTITCQASQDISNYLAWYQQKPGRAPTLLIFA
ASNLQSGVPSRFSGSGSGTEFTLTISGLQPEDFATYYCLQDSSYPPTFGG
GTKVDIK
SEQ ID NO: 70
DVVMTQSPSTLSASVGDRVTITCRASQSISSWLAWYQQKPGRAPTLLIYK
ASNLQSGVPSRFSGSGSGTEFTLTISSLQPDDFASYYCQQYSNYPLTFGG
GTKVDIK
SEQ ID NO: 71
DVVMTQSPSFLSASVGDRVTITCRASHGISNYFAWYQQKPGKAPKLLIYA
TSTLQSGVPSRFSGSGSGTEFTLTISGLQPEDFATYYCQQYSSYPLTFGG
GTKVDIK
SEQ ID NO: 72
DVVMTQSPSTLSAYVGDRITITCRASRGSNYLAWYQQKPGKAPKLLIYAT
STLQSGVPLRFSGSGSGTEFTLTISGLQPEDFATYYCQQYDSYPPTFGGG
TKVDIK

The antigen binding domain may comprise SEQ ID NO: 34, or 73-86 or a variant which is at least 80% identical to SEQ ID NO: 34, or 73-86 and is capable of binding to HLA-A2. The variant which may be at least 85, 90, 95, 97, 98 or 99% identical to SEQ ID NO: 34, or 73-86.

SEQ ID NO: 73
QVQLVOSGGGVVQPGGSLRVSCAASGVTLSDYGMHWVRQAPGKGLEWVAF
IRNDGSDKYYADSVKGRFTISRDNSEKTVSLQMSSLRAEDTAVYYCAKNG
ESGPLDYWYLDLWGSSGGGGSGGGGSGGGGSDVVMTQSPSSLSASVGDRV
TITCQSSLDISHYLNWYQQKPGKAPKLLIYDASNLETGVPSRFSGSGSGT
HFTFTISSLQPEDFATYYCQQYDNLPLTFGGGTKLEIK
SEQ ID NO: 34
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTLVTVSSGGGGSGGGGSGGGGSDVVMTQSPSSLS
ASVGDRVTITCQASQDISNYLNWYQQKPGKAPKLLIYDASNLETGVPSRF
SGSGSGTDFTFTISSLQPEDIATYYCQQYDNLPPTFGGGTKLTVLG
SEQ ID NO: 74
QVQLVQSGGGVVQPGGSMRVSCAASGVTLSDYGMHWVRQAPGKGLEWVAF
IRNDGSDKYYADSVRGRFTISRDNSKKTVFLQMNSLRAEDTAVYYCAKNG
ESGPLDYWYFDLWGRGTSSGGGGSGGGGSGGGGSDIVLMQSPSFLSASVG
DRVTITCRASHGINNYLAWYQQKPGKAPKLLIYAASTLQSGVPSRFSGSG
SGTEFTLTISSLQPEDFATYYCQQYDSYPPTFGRTKVEIKR
SEQ ID NO: 75
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDFATYYCQQYSSFPLTFGGGTKVDIK
SEQ ID NO: 76
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQEPGKAPKLLIYDETHLDSGVPSRFTGSR
SGTDFTLTISSLQPEDFATYYCQQYDSLPPTFGGGTKVDIK
SEQ ID NO: 77
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDIATYYCQQYDNLPITFGGGTKVDIK
SEQ ID NO: 78
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDIATYYCQQYDNLPSTFGGGTKVDIK
SEQ ID NO: 79
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDFGTYYCQQYNTYPLTFGGGTKVDIK
SEQ ID NO: 80
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLTASVG
DRVTITCQASQDISNYLNWYQQKPGKAPKLLIYDASNLETGVPSRFSGSG
SGTDFTLSIDSLQPEDFATYYCQQYHTYPLTFGGGTKVDIK
SEQ ID NO: 81
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDIATYYCQQYDNLPLTFGGGTKVDIK
SEQ ID NO: 82
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCRTSQGISSALAWYQQKPGKAPKLLIYDASSLESGVPSRFSGSG
SGTDFTLTISSLQPEDFATYYCQQFNNYPLTFGGGTKVDIK
SEQ ID NO: 83
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLAWYQQKPGRAPTLLIFAASNLQSGVPSRFSGSG
SGTEFTLTISGLQPEDFATYYCLQDSSYPPTFGGGTKVDIK
SEQ ID NO: 84
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSTLSASVG
DRVTITCRASQSISSWLAWYQQKPGRAPTLLIYKASNLQSGVPSRFSGSG
SGTEFTLTISSLQPDDFASYYCQQYSNYPLTFGGGTKVDIK
SEQ ID NO: 85
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSFLSASVG
DRVTITCRASHGISNYFAWYQQKPGKAPKLLIYATSTLQSGVPSRFSGSG
SGTEFTLTISGLQPEDFATYYCQQYSSYPLTFGGGTKVDIK
SEQ ID NO: 86
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSTLSAYVG
DRITITCRASRGSNYLAWYQQKPGKAPKLLIYATSTLQSGVPLRFSGSGS
GTEFTLTISGLQPEDFATYYCQQYDSYPPTFGGGTKVDIK

The antigen binding domain may comprise SEQ ID NO: 73, or a variant which is at least 80% identical to SEQ ID NO: 73. Suitably, the variant may be at least 85, 90, 95, 97, 98 or 99% identical to SEQ ID NO: 73.

The antigen binding domain may comprise SEQ ID NO: 34, or a variant which is at least 80% identical to SEQ ID NO: 34. Suitably, the variant may be at least 85, 90, 95, 97, 98 or 99% identical to SEQ ID NO: 34.

The antigen binding domain may comprise SEQ ID NO: 74, or a variant which is at least 80% identical to SEQ ID NO: 74. Suitably, the variant may be at least 85, 90, 95, 97, 98 or 99% identical to SEQ ID NO: 74.

The antigen binding domain may comprise SEQ ID NO: 75, or a variant which is at least 80% identical to SEQ ID NO: 75. Suitably, the variant may be at least 85, 90, 95, 97, 98 or 99% identical to SEQ ID NO: 75.

The antigen binding domain may comprise SEQ ID NO: 76, or a variant which is at least 80% identical to SEQ ID NO: 76. Suitably, the variant may be at least 85, 90, 95, 97, 98 or 99% identical to SEQ ID NO: 76.

The antigen binding domain may comprise SEQ ID NO: 77, or a variant which is at least 80% identical to SEQ ID NO: 77. Suitably, the variant may be at least 85, 90, 95, 97, 98 or 99% identical to SEQ ID NO: 77.

The antigen binding domain may comprise SEQ ID NO: 78, or a variant which is at least 80% identical to SEQ ID NO: 78. Suitably, the variant may be at least 85, 90, 95, 97, 98 or 99% identical to SEQ ID NO: 78.

The antigen binding domain may comprise SEQ ID NO: 79, or a variant which is at least 80% identical to SEQ ID NO: 79. Suitably, the variant may be at least 85, 90, 95, 97, 98 or 99% identical to SEQ ID NO: 79.

The antigen binding domain may comprise SEQ ID NO: 80, or a variant which is at least 80% identical to SEQ ID NO: 80. Suitably, the variant may be at least 85, 90, 95, 97, 98 or 99% identical to SEQ ID NO: 80.

The antigen binding domain may comprise SEQ ID NO: 81, or a variant which is at least 80% identical to SEQ ID NO: 81. Suitably, the variant may be at least 85, 90, 95, 97, 98 or 99% identical to SEQ ID NO: 81.

The antigen binding domain may comprise SEQ ID NO: 82, or a variant which is at least 80% identical to SEQ ID NO: 82. Suitably, the variant may be at least 85, 90, 95, 97, 98 or 99% identical to SEQ ID NO: 82.

The antigen binding domain may comprise SEQ ID NO: 83, or a variant which is at least 80% identical to SEQ ID NO: 83. Suitably, the variant may be at least 85, 90, 95, 97, 98 or 99% identical to SEQ ID NO: 83.

The antigen binding domain may comprise SEQ ID NO: 84, or a variant which is at least 80% identical to SEQ ID NO: 84. Suitably, the variant may be at least 85, 90, 95, 97, 98 or 99% identical to SEQ ID NO: 84.

The antigen binding domain may comprise SEQ ID NO: 85, or a variant which is at least 80% identical to SEQ ID NO: 85. Suitably, the variant may be at least 85, 90, 95, 97, 98 or 99% identical to SEQ ID NO: 85.

The antigen binding domain may comprise SEQ ID NO: 86, or a variant which is at least 80% identical to SEQ ID NO: 86. Suitably, the variant may be at least 85, 90, 95, 97, 98 or 99% identical to SEQ ID NO: 86.

In another embodiment, the antigen binding domain may comprise SEQ ID NO:. 196 or a variant which is at least 80% identical to SEQ ID NO: 196. Suitably, the variant may be at least 85, 90, 95, 97, 98 or 99% identical to SEQ ID NO: 1096. SEQ ID NO: 196 is the scFv sequence used in constructs CIV to CIX in Example 10 below.

SEQ ID NO: 196
QVQLVQSGGGVVQPGGSLRVSCAASGVTLSDYGMHWVRQAPGKGLEW
VAFIRNDGSDKYYADSVKGRFTISRDNSEKTVSLQMSSLRAEDTAVY
YCAKNGESGPLDYWYLDLWGRGT [symbol] DV
VMTQSPSSLSASVGDRVTITCQSSLDISHYLNWYQQKPGKAPKLLIY
DASNLETGVPSRFSGSGSGTHFTFTISSLQPEDFATYYCQQYDNLPL
TFGGGTKLEIK

### Pharmaceutical Composition

There is also provided a pharmaceutical composition comprising an engineered cell, (e.g., Treg) of the invention.

A pharmaceutical composition is a composition that comprises or consists of a therapeutically effective amount of a pharmaceutically active agent i.e. the cell (e.g., T cell (Treg)). It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the Treg and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The cells (e.g., Tregs) or pharmaceutical compositions according to the present invention may be administered in a manner appropriate for treating and/or preventing the disease described herein. The quantity and frequency of administration will be determined by such factors as the condition of the subject, and the type and severity of the subjects' disease, although appropriate dosages may be determined by clinical trials. The pharmaceutical composition may be formulated accordingly.

The cell (e.g., Treg) or pharmaceutical composition as described herein can be administered parenterally, for example, intravenously, or they may be administered by infusion techniques. The cell (e.g., Treg) or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise cells (e.g., Tregs) of the invention in infusion media, for example sterile isotonic solution. The pharmaceutical composition may be enclosed in ampoules, disposable syringes or multiple dose vials made of glass or plastic.

The cell (e.g., Treg) or pharmaceutical composition may be administered in a single or in multiple doses. Particularly, the cell (e.g., Treg) or pharmaceutical composition may be administered in a single, one off dose. The pharmaceutical composition may be formulated accordingly.

The pharmaceutical composition may further comprise one or more active agents.

The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18).

Depending upon the disease and subject to be treated, as well as the route of administration, the cell (e.g., Treg) or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for cells (e.g., Tregs) of the invention, doses of 5×10⁷ to 3×10⁹ cells, or 10⁸ to 2×10⁹ cells per subject may be administered.

The cell (e.g., Treg) may be appropriately modified for use in a pharmaceutical composition. For example, cells, (e.g., Tregs) may be cryopreserved and thawed at an appropriate time, before being infused into a subject.

The invention further includes the use of kits comprising the cell (e.g., Treg) and/or pharmaceutical composition of the present invention. Preferably said kits are for use in the methods and uses as described herein, e.g., the therapeutic methods as described herein. Preferably said kits comprise instructions for use of the kit components.

### Method of Treatment

The present invention provides a method for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation (e.g., chronic inflammation secondary to metabolic disorders) which comprises the step of administering an engineered cell (e.g., Treg) or a pharmaceutical composition of the invention to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

For example, the engineered cells (e.g., Tregs) may be administered to a subject with a disease in order to lessen, reduce, or improve at least one symptom of disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The engineered cells (e.g., Tregs) may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells (e.g., Tregs) are not administered, or progression of the disease may be completely stopped.

In one embodiment, the subject is a transplant recipient undergoing immunosuppression therapy.

Suitably, the subject is a mammal. Suitably, the subject is a human.

The transplant may be selected from a liver, kidney, heart, lung, pancreas, intestine, stomach, bone marrow, vascularized composite tissue graft, and skin transplant.

Suitably, the CAR may comprise an antigen binding domain which is capable of specifically binding to a HLA antigen that is present in the graft (transplant) donor but not in the graft (transplant) recipient.

Suitably, the transplant is a liver transplant. In embodiments where the transplant is a liver transplant, the antigen may be a HLA antigen present in the transplanted liver but not in the patient, a liver-specific antigen such as NTCP, or an antigen whose expression is up-regulated during rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

Suitably, the antigen may be HLA-A2.

The present invention further provides a method for treating and/or preventing graft-versus-host disease (GvHD), an autoimmune or allergic disease; or to promote tissue repair and/or tissue regeneration; or to ameliorate inflammation (e.g., chronic inflammation secondary to metabolic disorders).

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

Suitably, treating and/or preventing cellular and/or humoral transplant rejection may refer to administering an effective amount of the cells (e.g. Tregs) of the invention such that the amount of immunosuppressive drugs that a transplant recipient requires is reduced, or may enable the discontinuation of immunosuppressive drugs.

Preventing a disease relates to the prophylactic use of the cells of the present invention. In this respect, the cells may be administered to a subject who has not yet contracted the disease and/or who is not showing any symptoms of the disease to prevent the disease or to reduce or prevent development of at least one symptom associated with the disease. The subject may have a predisposition for, or be thought to be at risk of developing, the disease.

The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis; neurodegenerative disease, for example, amyotrophic lateral sclerosis (ALS); chronic inflammatory demyelinating polyneuropathy (CIPD) and juvenile onset diabetes. Particularly, the autoimmune condition is Type I Diabetes.

Suitably, the therapeutic methods of the invention may comprise the step of administering an engineered cell (e.g. Treg) according to the invention, or obtainable (e.g. obtained) by a method according to the present invention, or a nucleic acid molecule/polynucleotide or a vector as defined herein (for example in a pharmaceutical composition as described above) to a subject.

Suitably, the present methods for treating and/or preventing a disease may comprise administering a cell (e.g. an engineered Treg) according to the present invention (for example in a pharmaceutical composition as described above) to a subject.

The method may involve the steps of:

(i) isolating a cell-containing sample or providing a cell-containing sample;
(ii) introducing a nucleic acid molecule or a vector as defined herein to the cell; and
(iii) administering the cells from (ii) to a subject.

Suitably, the cell is a Treg as defined herein.

Suitably, an enriched Treg population may be isolated and/or generated from the cell containing sample prior to, and/or after, step (ii) of the method.

For example, isolation and/or generation may be performed prior to and/or after step (ii) to isolate and/or generate an enriched Treg sample. Enrichment may be performed after step (ii) to enrich for cells and/or Tregs comprising the chimeric receptor (e.g., CAR), the polynucleotide, and/or the vector as described herein.

Suitably, the nucleic acid molecule or vector may be introduced by transduction. Suitably, the nucleic acid molecule or vector may be introduced by transfection.

Suitably, the cell may be autologous. Suitably, the cell may be allogenic.

Suitably, the cell (e.g. the engineered Treg) may be administered is combination with one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18). The engineered Treg may be administered simultaneously with or sequentially with (i.e. prior to or after) the one or more other therapeutic agents.

Suitably the subject is a mammal. Suitably the subject is a human.

Tregs may be activated and/or expanded prior to, or after, the introduction of a nucleic acid molecule encoding the CAR as described herein, for example by treatment with an anti-CD3 monoclonal antibody or both anti-CD3 and anti-CD28 monoclonal antibodies.

The Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ.

As used herein “activated” means that a Treg or population of Tregs has been stimulated, causing the Treg(s) to proliferate. As used herein “expanded” means that a Treg or population of Tregs has been induced to proliferate. The expansion of a population of Tregs may be measured for example by counting the number of Tregs present in a population. The phenotype of the Tregs may be determined by methods known in the art such as flow cytometry.

The Tregs may be washed after each step of the method, in particular after expansion.

The population of engineered Treg cells according to the present invention may be further enriched by any method known to those of skill in the art, for example by FACS or magnetic bead sorting.

The steps of the method of production may be performed in a closed and sterile cell culture system.

### Nucleic Acid Molecules/Polynucleotides

Nucleic acid molecules and polynucleotides as defined herein may comprise DNA or RNA. They may be single-stranded or double-stranded. It will be understood by a skilled person that numerous different nucleic acid molecules/polynucleotides can encode the same polypeptide as a result of the degeneracy of the genetic code. In addition, it is to be understood that the skilled person may, using routine techniques, make nucleotide substitutions that do not affect the polypeptide sequence encoded by the nucleic acid molecules/polynucleotides as defined herein to reflect the codon usage of any particular host organism in which the polypeptides of the invention are to be expressed.

The nucleic acid molecules/polynucleotides may be modified by any method available in the art. Such modifications may be carried out in order to enhance the in vivo activity or lifespan of the nucleic acid molecules/polynucleotides as defined herein.

Nucleic acid molecules/polynucleotides such as DNA nucleic acid molecules/polynucleotides may be produced recombinantly, synthetically or by any means available to those of skill in the art. They may also be cloned by standard techniques.

Longer nucleic acid molecules/polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The present nucleic acid molecule/polynucleotide may further comprise a nucleic acid sequence encoding a selectable marker. Suitably selectable markers are well known in the art and include, but are not limited to, fluorescent proteins—such as GFP. Suitably, the selectable marker may be a fluorescent protein, for example GFP, YFP, RFP, tdTomato, dsRed, or variants thereof. In some embodiments the fluorescent protein is GFP or a GFP variant. The nucleic acid sequence encoding a selectable marker may be provided in combination with a nucleic acid sequence encoding the present chimeric receptor (e.g., CAR) in the form of a nucleic acid construct. Such a nucleic acid construct may be provided in a vector.

Suitably, the selectable marker/reporter domain may be a luciferase-based reporter, a PET reporter (e.g. Sodium Iodide Symporter (NIS)), or a membrane protein (e.g. CD34, low-affinity nerve growth factor receptor (LNGFR)).

The nucleic acid sequences encoding different polypeptides as described herein, e.g. the chimeric receptor (e.g., CAR) and the FOXP3 (and/or additionally or alternatively, one or more selectable markers) may be separated by one or more co-expression sites which enables expression of each polypeptide as a discrete entity. Suitable co-expression sites are known in the art and include, for example, internal ribosome entry sites (IRES) and self-cleaving peptides. Particularly, the first polynucleotide sequence encoding FOXP3 and the second polynucleotide sequence encoding the chimeric receptor (e.g., CAR) may be separated by a co-expression site encoding a self-cleaving peptide.

Further suitable co-expression sites/sequences include self-cleaving or cleavage domains. Such sequences may either auto-cleave during protein production or may be cleaved by common enzymes present in the cell. Accordingly, inclusion of such self-cleaving or cleavage domains in the polypeptide sequence enables a first and a second polypeptide to be expressed as a single polypeptide, which is subsequently cleaved to provide discrete, separated functional polypeptides.

A self-cleavage sequence allows the polypeptides to be expressed and/or produced as separate, or discrete components. By this it is meant that, although the polypeptides are encoded by a single nucleic acid molecule, through “cleavage” during or after translation at the encoded cleavage sites, they are expressed or produced as separate polypeptides, and thus at the end of the protein production process in the cell, they are present in the cell as separate entities, or separate polypeptide chains. Self-cleaving peptide sequences include particularly 2A and 2A-like peptides. Although described as “self-cleaving”, such peptides are believed to function by allowing ribosome skipping such that a peptide bond is not formed (skipped) at the C-terminus of the 2A sequence, leading to separation of the 2A sequence and the next polypeptide downstream of it. The term “cleavage” as used herein thus includes the skipping of peptide bond formation.

Suitable self-cleaving or cleavage domains include, but are not limited to, those shown as SEQ ID NO: 46-51.

(SEQ ID NO: 46) P2A peptide-cleavage domain:
GSGATNFSLLKQAGDVEENPGP
(SEQ ID NO: 47) T2A peptide-cleavage domain:
GSGEGRGSLLTCGDVEENPGP
(SEQ ID NO: 48) E2A peptide-cleavage domain:
GSGQCTNYALLKLAGDVESNPGP
(SEQ ID NO: 49) F2A peptide-cleavage domain:
GSGVKQTLNFDLLKLAGDVESNPGP
(SEQ ID NO: 50) Furin site-cleavage domain:
RXXR
(preferentially: RRKR-SEQ ID NO: 51)

The above 2A sequences may be modified to remove the three amino acids GSG at the N-terminus. Thus, also included as options are sequences corresponding to SEQ ID NO: 46-49 but without the N-terminal GSG.

In an embodiment, a P2A self-cleaving peptide of SEQ ID NO: 46 without the N-terminal GSG may be used in the context of a furin cleavage site of SEQ ID NO: 197 IDGSG.

The use of a selectable marker is advantageous as it allows cells (e.g. Tregs) in which a polynucleotide or vector of the present invention has been successfully introduced (such that the encoded chimeric receptor (e.g., CAR) is expressed) to be selected and isolated from a starting cell population using common methods, e.g. flow cytometry.

### Codon Optimisation

The nucleic acid molecule/polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### Promoter

The first polynucleotide sequence and the second polynucleotide sequence as described herein are operably linked to the same promoter. A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person. Particular promoters include EFS (or functional truncations thereof), SSFV, PGK, and CMV. “Operably linked to the same promoter” means that transcription of the polynucleotide sequences may be initiated from the same promoter (e.g. transcription of the first and second polynucleotide sequences is initiated from the same promoter) and that the polynucleotide sequences are positioned and oriented for transcription to be initiated from the promoter. Polynucleotides operably linked to a promoter are under transcriptional regulation of the promoter.

### Vectors

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter (as described above) for the expression of a nucleic acid molecule/polynucleotide and optionally a regulator of the promoter.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal.

Suitably, the vector of the invention may include a 5′LTR and a 3′LTR.

The vector of the invention may comprise one or more additional regulatory sequences which may act pre- or post-transcriptionally. “Regulatory sequences” are any sequences which facilitate expression of the polypeptides, e.g. act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g. operably linked to the promoter.

Vectors comprising nucleic acid molecules/polynucleotides of the invention may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a polynucleotide of the invention.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Multiple vectors, e.g. encoding different CARs as defined herein, or encoding a CAR as defined herein and a further polypeptide could be used for transduction/transfection.

### Method of Making a Cell

Engineered cells (e.g., Tregs) of the present invention may be generated by introducing a nucleic acid molecule or vector as defined herein, by one of many means including transduction with a viral vector, and transfection with DNA or RNA.

The cell of the invention may be made by: introducing to a cell (e.g. by transduction or transfection) the nucleic acid molecule/polynucleotide or vector as defined herein.

Suitably, the cell may be from a sample isolated from a subject.

The engineered cell (e.g., Treg) of the present invention may be generated by a method comprising the following steps:
(i) isolation of a cell-containing sample from a subject or provision of a cell-containing sample; and
(ii) transduction or transfection of the cell-containing sample with a polynucleotide, a nucleic acid, or a vector as defined herein, to provide a population of engineered cells.

Suitably, a Treg-enriched sample may be isolated from, enriched, and/or generated from the cell-containing sample prior to and/or after step (ii) of the method. For example, isolation, enrichment and/or generation of Tregs may be performed prior to and/or after step (ii) to isolate, enrich or generate a Treg-enriched sample. Isolation and/or enrichment from a cell-containing sample may be performed after step (ii) to enrich for cells and/or Tregs comprising the chimeric receptor (e.g., CAR), the nucleic acid molecule/polynucleotide, and/or the vector as described herein.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example from Tcon cells by introducing DNA or RNA coding for FOXP3 and/or from ex-vivo differentiation of inducible progenitor cells or embryonic progenitor cells.

Suitably, the cell is a Treg as defined herein.

Suitably, the engineered Treg of the present invention may be generated by a method comprising the following steps:

(i) isolation of a Treg-enriched sample from a subject or provision of a Treg-enriched sample; and
(ii) transduction or transfection of the Treg-enriched sample with a polynucleotide, a nucleic acid, or a vector as defined herein, to provide a population of engineered Treg cells according to the present invention.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

### Statements

1. A nucleic acid comprising (i) a first polynucleotide sequence encoding FOXP3 and (ii) a second polynucleotide sequence encoding a chimeric receptor (e.g. a chimeric antigen receptor (CAR)) wherein said chimeric receptor (e.g., CAR) comprises an endodomain comprising a STAT5 association motif, a JAK1 and/or JAK2 binding motif and a JAK3 binding motif, wherein said first and second polynucleotide sequences are operably linked to the same promoter.
2. The nucleic acid of clause 1 wherein the chimeric receptor (e.g., CAR) endodomain comprises two or more STAT5 association motifs.
3. The nucleic acid of clause 1 or 2 wherein the one or more STAT5 association motifs is from an interleukin receptor (IL) receptor endodomain.
4. The nucleic acid of any one of clauses 1 to 3 wherein the one or more STAT5 association motifs is from IL2Rβ, IL7Rα, (CSF2RB), IL-9R, IL-17R13, erythropoietin receptor, thrombopoietin receptor, growth hormone receptor and prolactin receptor.
5. The nucleic acid molecule of any one of clauses 1 to 4 wherein the STAT5 association motif comprises the amino acid motif YXXF/L (SEQ ID NO: 8); wherein X is any amino acid.
6. The nucleic acid molecule of any one of clauses 1 to 5 wherein the STAT5 association motif comprises one or more of the amino acid motifs YCTF (SEQ ID NO: 9), YFFF (SEQ ID NO: 10), YLSL (SEQ ID NO: 11), and/or YLSLQ (SEQ ID NO: 12).
7. The nucleic acid molecule of clause 6 wherein the STAT5 association motif comprises the amino acid motif YLSLQ (SEQ ID NO: 12).
8. The nucleic acid molecule of clause 7 wherein the endodomain comprises a first STAT5 association motif comprising the amino acid motif YLSLQ (SEQ ID NO: 12) and a second STAT5 association motif comprising the amino acid motif YCTF (SEQ ID NO: 9) or YFFF
9. The nucleic acid molecule of clause 8 wherein the endodomain comprises the following STAT5 association motifs: YLSLQ (SEQ ID NO: 12), YCTF (SEQ ID NO: 9) and YFFF (SEQ ID NO: 10).
10. The nucleic acid molecule of any one of clauses 1 to 9 wherein the JAK binding motif is a JAK1 binding motif
11. The nucleic acid molecule of clause 10 wherein the JAK1 binding motif is from an interleukin receptor (IL) receptor endodomain.
12. The nucleic acid molecule of any one of clauses 1 to 11 wherein the JAK1 binding motif comprises an amino acid motif shown as any one of SEQ ID NO: 13-19 or a variant which has at least 80% identity to SEQ ID NO: 13-19.
13. The nucleic acid molecule of clause 12 wherein the JAK1 binding motif is the amino acid motif shown as SEQ ID NO: 13; or a variant which has at least 80% identity to SEQ ID NO: 13.
14. The nucleic acid molecule of any one of clauses 1 to 13 wherein the CAR endodomain comprises an IL2Rβ endodomain shown as SEQ ID NO: 1; or a variant which has at least 80% sequence identity to SEQ ID NO: 1.
15. The nucleic acid molecule of any one of clauses 1 to 14 wherein the chimeric receptor (e.g., CAR) endodomain comprises a truncated IL2Rβ endodomain shown as any one of SEQ ID NO: 23 or 24; or a variant of SEQ ID NO: 23 or 24 which has at least 80% sequence identity thereto.
16. The nucleic acid molecule of any one of clauses 1 to 15 wherein the JAK3 binding motif comprises SEQ ID NO: 25 or 26 or a variant which has at least 80% sequence identity to SEQ ID NO: 25 or 26.
17. A nucleic acid molecule of any one of clauses 1 to 16 wherein the chimeric receptor (e.g., CAR) endodomain comprises SEQ ID NO: 45, 53 or 154; or a variant which has at least 80% sequence identity to SEQ ID NO: 45, 53 or 154.
18. A nucleic acid molecule of any one of clauses 1 to 17 wherein the JAK3 binding motif is positioned N terminal to the STAT5 association motif and JAK1 and/or JAK2 binding motifs in the chimeric receptor (e.g., CAR) endodomain.
19. A nucleic acid molecule of any one of clauses 1 to 18, wherein the JAK3 binding motif is connected to the STAT5 association motif and JAK1 and/or JAK2 binding motif through a linker or hinge.
20. A nucleic acid molecule of any one of clauses 1 to 19, wherein said nucleic acid molecule comprises a co-expression site between said first and said second polynucleotides.
21. A vector comprising the nucleic acid molecule of any one of clauses 1 to 20.
22. An engineered Treg cell comprising the nucleic acid molecule of any one of clauses 1 to 20 or the vector of clause 21.
23. A pharmaceutical composition comprising the engineered Treg cell of clause 22.
24. An engineered Treg cell of clause 22 or a pharmaceutical composition of clause 23 for use in therapy.
25. An engineered Treg cell of clause 22 for use in induction of tolerance to a transplant; treating and/or preventing graft-versus-host disease (GvHD), an autoimmune or allergic disease; to promote tissue repair and/or tissue regeneration; or to ameliorate chronic inflammation secondary to metabolic disorders.
26. A pharmaceutical composition of clause 23 for use in induction of tolerance to a transplant; treating and/or preventing graft-versus-host disease (GvHD), an autoimmune or allergic disease; to promote tissue repair and/or tissue regeneration; or to ameliorate chronic inflammation secondary to metabolic disorders.
27. A method of inducing tolerance to a transplant; treating and/or preventing graft-versus-host disease (GvHD), an autoimmune or allergic disease; or to promote tissue repair and/or tissue regeneration; or to ameliorate chronic inflammation secondary to metabolic disorders which comprises the step of administering an engineered Treg as defined in any of clause 22 or a pharmaceutical composition comprising an engineered Treg as defined in clause 23 to a subject.
28. A method according to clause 27 which comprises the following steps:
(i) isolation or provision of a Treg-enriched cell sample from a subject;
(ii) transduction or transfection of the Treg cells with: a nucleic acid molecule of any one of clauses 1 to 20; or a vector of clause 21; and
(iii) administering the Treg cells from (ii) to the subject.
29. Use of an engineered Treg as defined in clause 22 in the manufacture of a medicament for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate chronic inflammation secondary to metabolic disorders.
30. An engineered Treg or pharmaceutical composition for use according to any of clauses 24 to 26; a method according to clause 27 or 28; or the use according to clause 29 wherein the subject is a transplant recipient undergoing immunosuppression therapy.
31. An engineered Treg or pharmaceutical composition for use; a method according to; or the use according to clause 30 wherein the transplant is selected from a liver, kidney, heart, lung, pancreas, intestine, stomach, bone marrow, vascularized composite tissue graft, and skin transplant.
32. An engineered Treg or pharmaceutical composition for use; a method; or the use according to clause 31 wherein the transplant is a liver transplant.
33. An engineered Treg or pharmaceutical composition for use; a method or the use according to clause 32 wherein the CAR comprises an antigen binding domain which is capable of specifically binding to an antigen selected from: a HLA antigen present in the transplanted liver but not in the recipient, a liver-specific antigen such as NTCP, or an antigen whose expression is up-regulated during rejection or tissue inflammation such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.
34. An engineered Treg or pharmaceutical composition for use; a method or the use according to clause 33 wherein the CAR comprises an antigen binding domain which is capable of specifically binding to a HLA antigen that is present in the graft donor but not in the graft recipient.
35. An engineered Treg or pharmaceutical composition for use; a method or the use according to clause 34 wherein the antigen is HLA-A2.
36. An engineered Treg or pharmaceutical composition for use; a method or the use according to clause 35 wherein the CAR comprises an antigen binding domain comprises SEQ ID NO: 34 or a variant of SEQ ID NO: 34 with at least 80% identity thereto.
37. An engineered Treg or pharmaceutical composition for use; a method or the use according to and of clauses 24 to 36 wherein the autoimmune or allergic disease is selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); CIPD, multiple sclerosis, neurodegenerative diseases (e.g. ALS) and juvenile onset diabetes.
38. A method of producing an engineered Treg, comprising the following steps:
(i) isolation of a cell-containing sample from a subject or provision of a cell-containing sample; and
(ii) transduction or transfection of the cell-containing sample with one or more nucleic acid molecules comprising (i) a first polynucleotide sequence encoding FOXP3 and (ii) a second polynucleotide sequence encoding a chimeric receptor (e.g., chimeric antigen receptor (CAR)) wherein said chimeric receptor (e.g., CAR) comprises an endodomain comprising a STAT5 association motif, a JAK1 and/or JAK2 binding motif and a JAK3 binding motif, or with one or more vectors comprising said nucleic acid molecule(s), to provide a population of engineered cells;
   wherein the cell-containing sample comprises Tregs and/or Tregs are enriched and/or generated from the cell-containing sample prior to or after step (ii).

The invention will now be further described by way of Examples, which are meant to serve to assist one of ordinary skill in the art in carrying out the invention and are not intended in any way to limit the scope of the invention.

### EXAMPLES

### Example 1—Generation of Anti-HLA.A2 IL2R CAR-Tregs

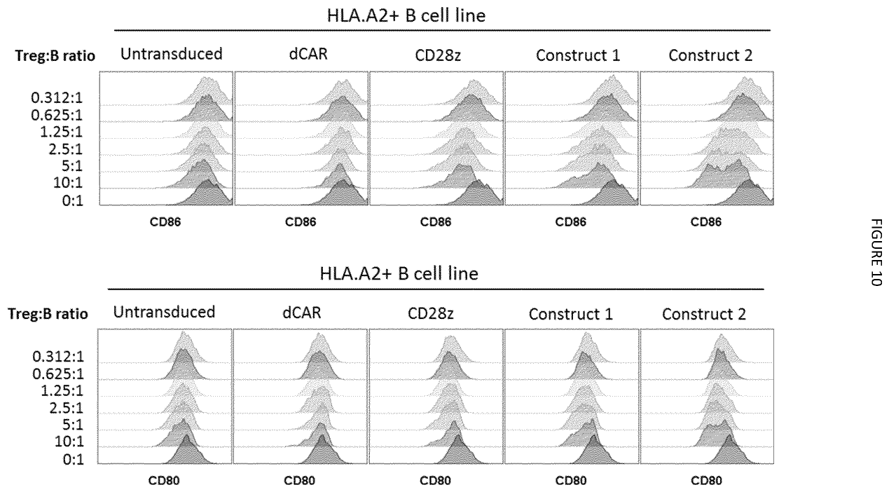

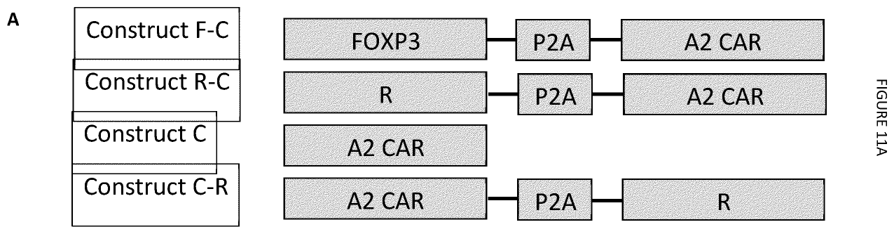

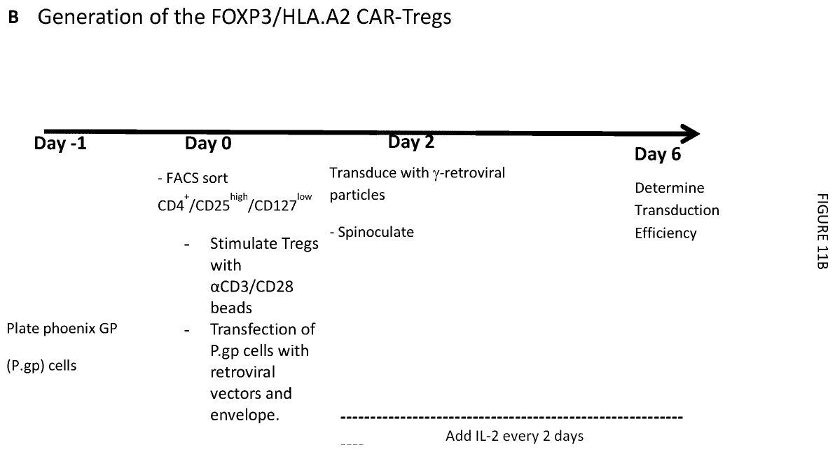

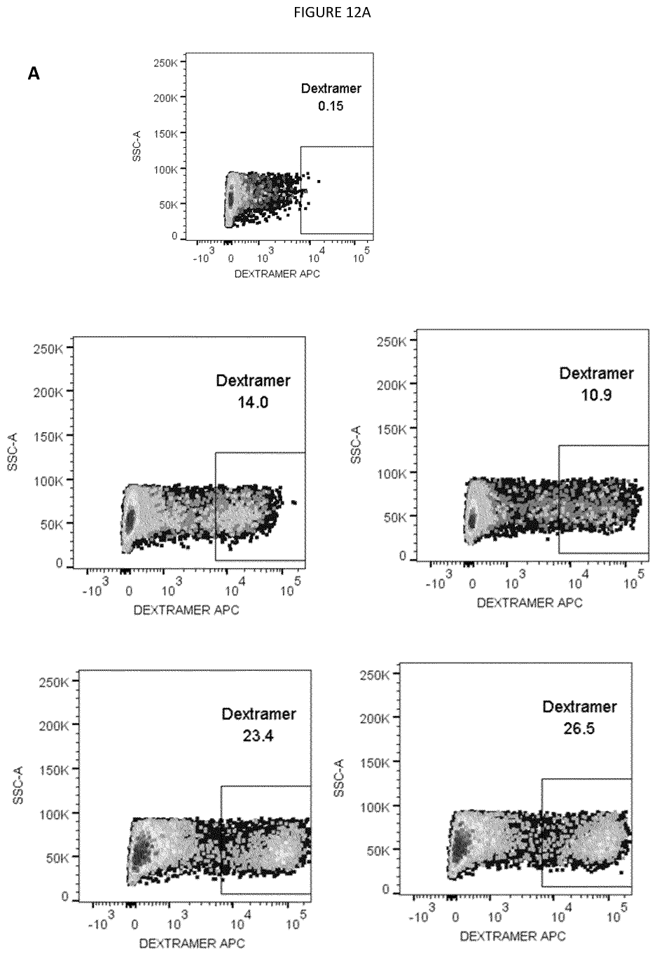

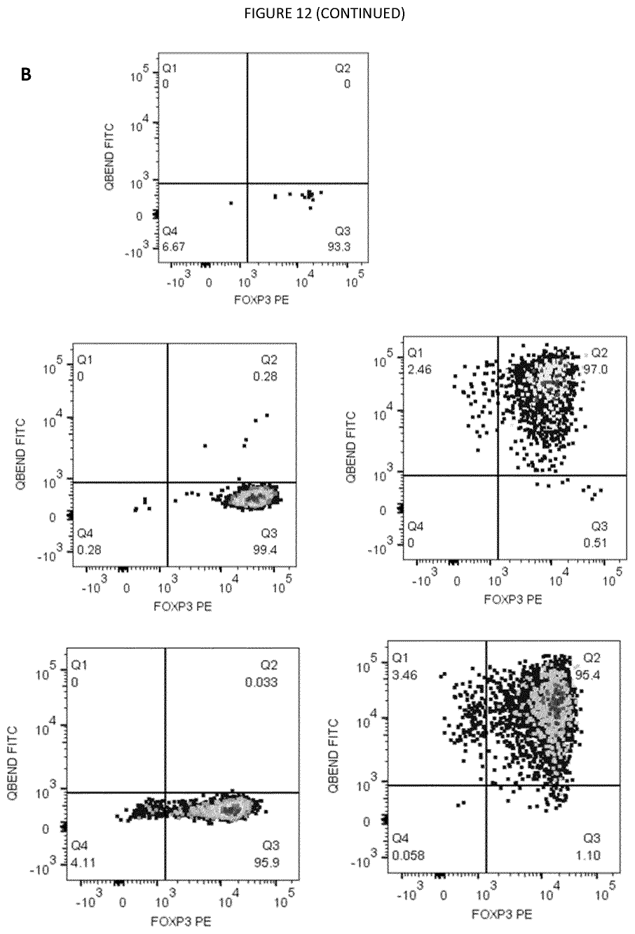

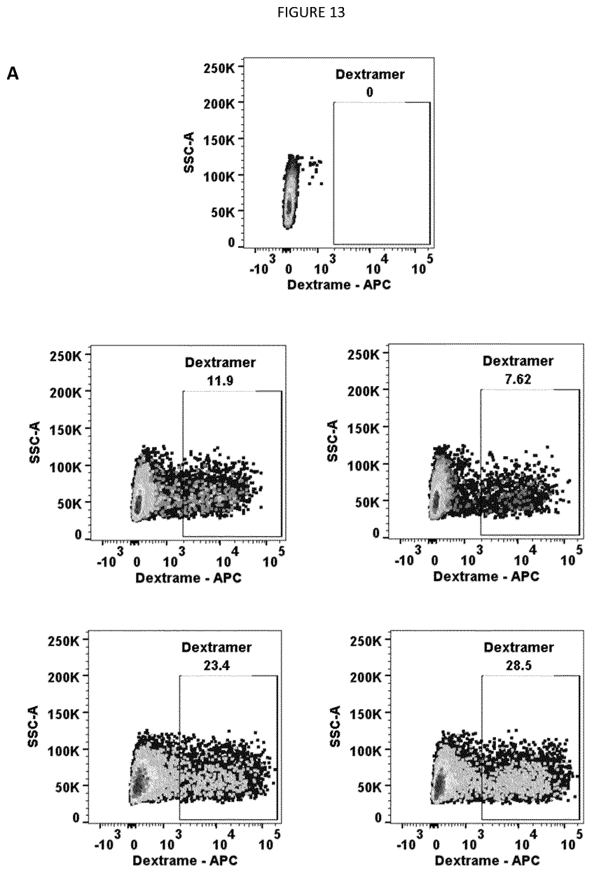

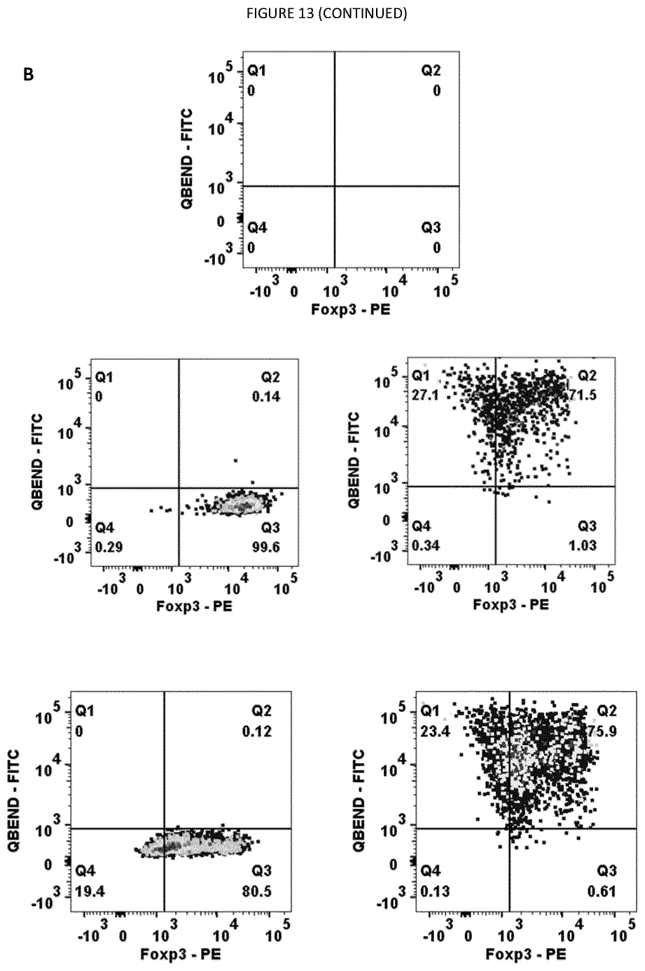

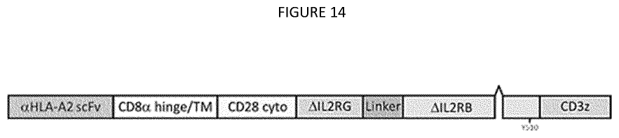

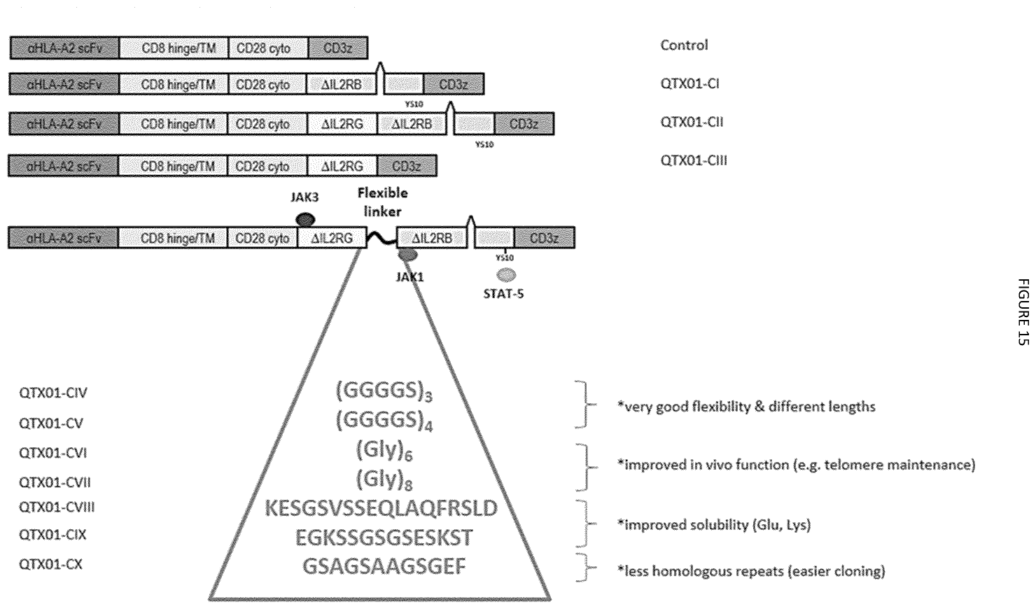

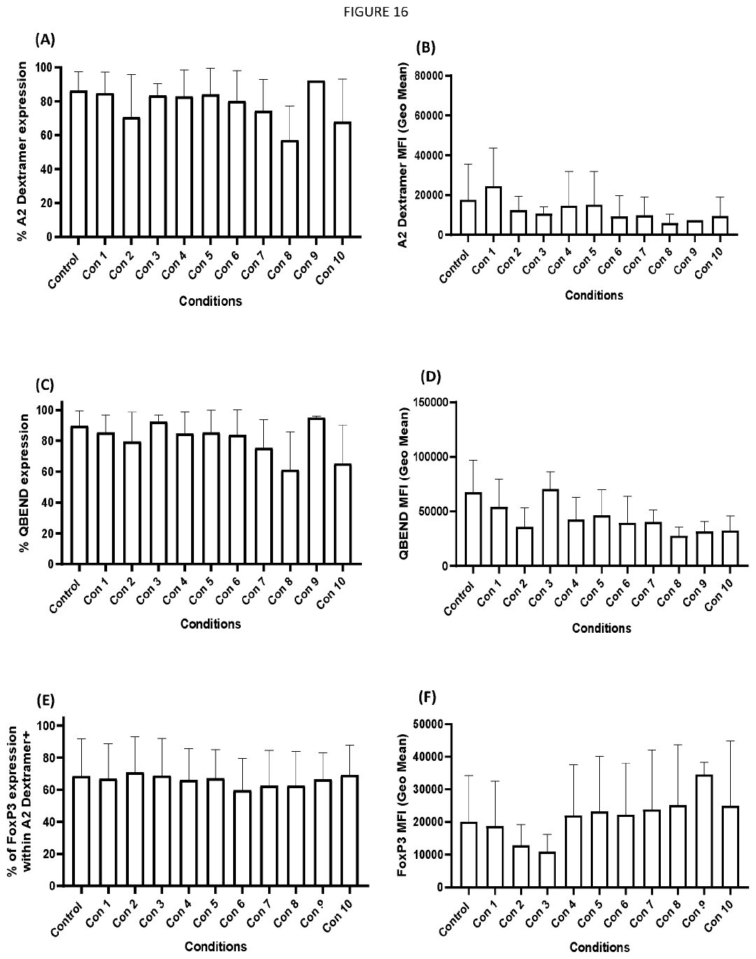

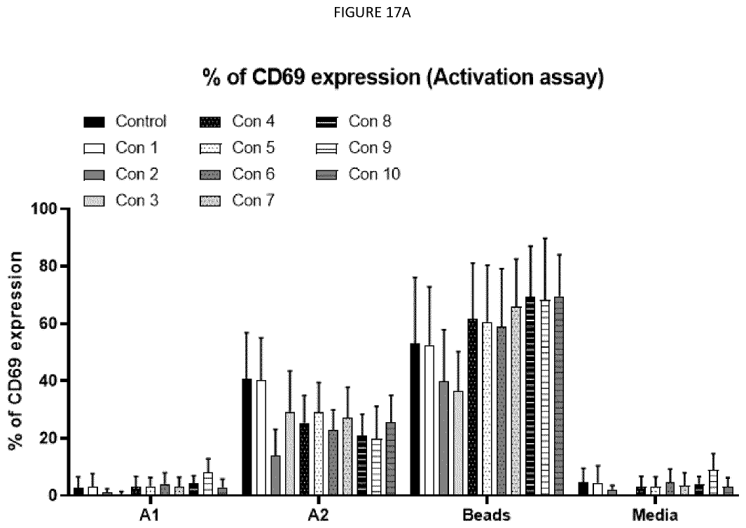

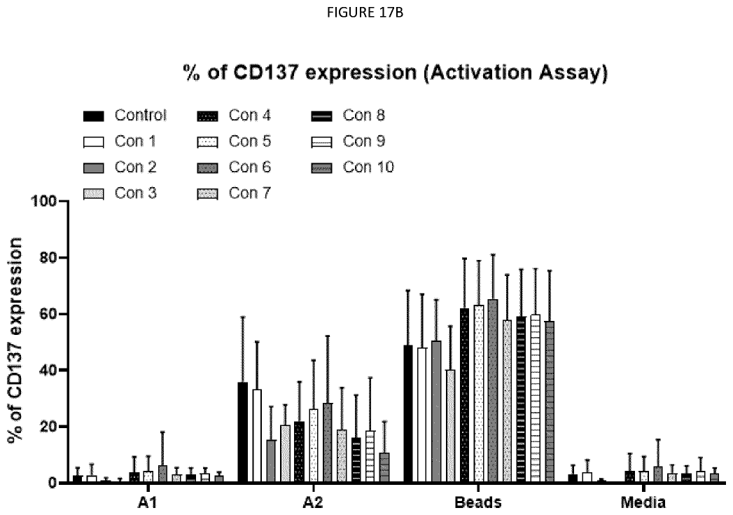

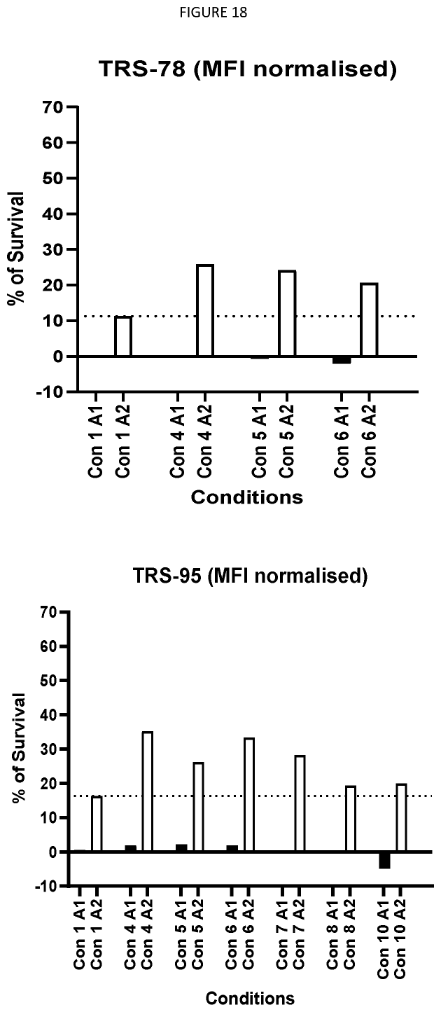

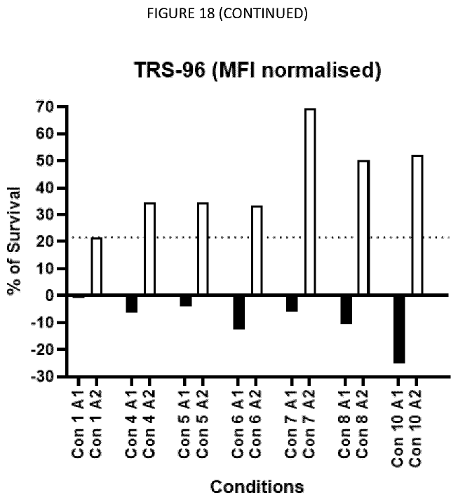

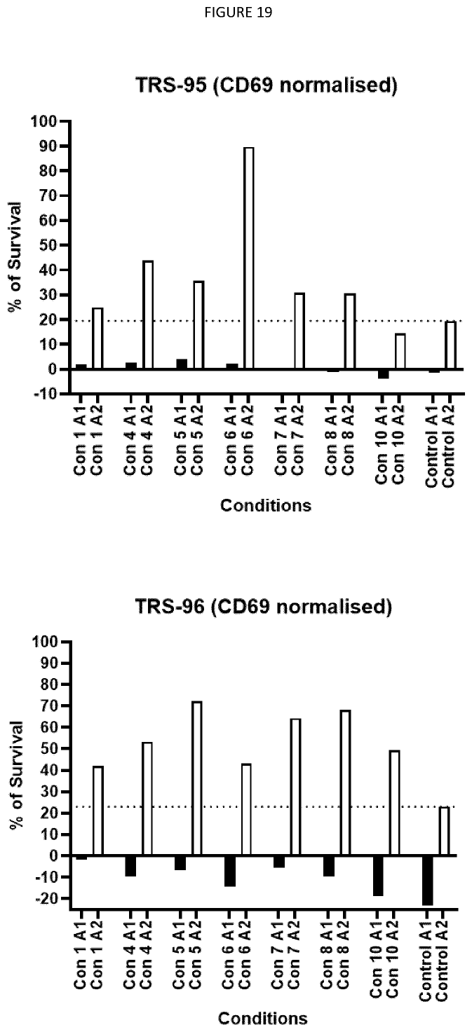

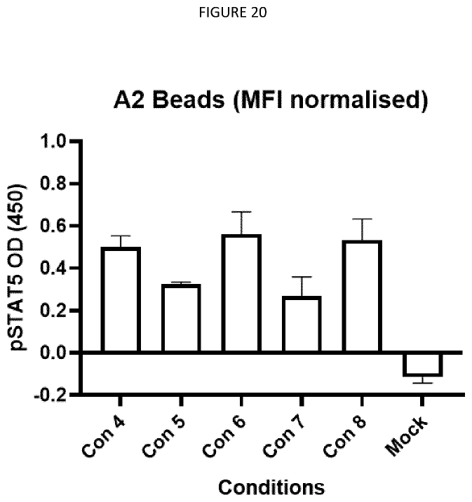

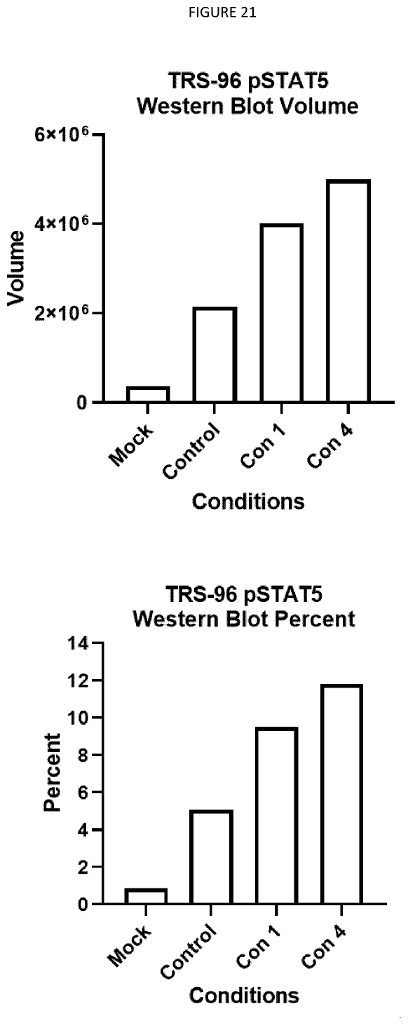

CD4+CD25hiCD127low cells were isolated and activated with anti-CD3/CD28 beads. Three days after activation Tregs were transduced with lentivirus containing the HLA.A2-CAR (shown in FIG. 2) and GFP reporter gene. Cellular expansion of total Tregs after polyclonal activation showed no significant differences between untransduced or transduced Treg (FIG. 3).

### Example 2—Quantification of Transduction Efficacy of Anti-HLA.A2 IL2R Constructs Over Time

GFP expression was analysed on Tregs untransduced and transduced with CAR constructs at different time points after cell activation.

Frequency of GFP+ cells was analysed to evaluate the transduction efficacy and the expression persistence of the different constructs over the Treg expansion period. Tregs containing dCAR, CD28z, Construct 1, 2 and 3 showed similar expression frequencies after transduction. The percentages of GFP+ cells among whole Tregs were maintained during polyclonal cellular expansion (FIG. 4).

### Example 3—Quantification of Cell Surface Expression of Anti-HLA.A2 IL2R CAR Constructs on Transduced Tregs

Membrane expression of CAR construct on untransduced and transduced Tregs was analysed by PE-conjugated HLA-A*0201/CINGVCWTV dextramers (Immudex, Copenhagen, Denmark). The frequency of Tregs expressing the CAR protein in the cell surface (HLA-A2 dextramer+) was similar between all the constructs (FIG. 5).

### Example 4—Phenotypic characterization of CAR Tregs after polyclonal cell expansion

Tregs were cultured and expanded for 15 days in the presence of anti-CD3/CD28 activation beads and IL-2. Treg related markers FOXP3, HELIOS, CTLA4 and TIGIT were analysed by FACS on untransduced and transduced Tregs to assess phenotypic lineage stability on day 15 of culture.

Untransduced and CAR-transduced showed similar expression levels of proteins associated with Treg lineage and function after polyclonal expansion (FIG. 6).

### Example 5—Evaluation of the Antigen-Specificity of Anti-HLA.A2 IL2R CAR Tregs

Untransduced and transduced Tregs were cultured for 18 hours in the presence of different stimulus. CD69 and CD137 activation markers were analysed to assess specific and unspecific cell activation.

Transduced Tregs with the CD28z, Construct 1, 2 and 3 CARs showed similar specificity for HLA-A2 molecules based on the expression of T cell activation markers. The expression of CD69 and CD137 was not increase on inactivated cells or after the culture with HLA-A1 expressing cells. The dCAR construct showed no activation due to the lack of signaling endodomains (FIG. 7).

### Example 6—STAT5 Phosphorylation Analysis as an Indicator of IL2R CAR Signaling

Transduced CAR Tregs were rested overnight in culture media without IL2. STAT5 phosphorylation of Tregs was assessed by FACS analysis 10 and 120 minutes after culture with media alone, 1000 IU/ml IL-2 or in the presence of HLA.A2-Ig based artificial APCs (produced following the protocol described at DOI: 10.3791/2801).

The integration of the IL2R endodomains into the CAR construct showed efficient phosphorylation of STAT5 after the CAR activation by HLA-A2 molecules. No significant increase of pSTAT5 was detected on CAR-Tregs without the IL2R endodomains after culture with HLA-A2 beads (FIG. 8).

### Example 7—Evaluation of Treg Survival after Unspecific and HLA.A2 Specific Activation in the Absence of IL-2

CAR transduced Tregs with different constructs were cultured with anti-CD3/28 activation beads and K562.A2 expression cells without the presence of IL-2. Cell survival was assessed 7 days after activation by FACS analysis.

Tregs expressing CAR constructs containing the IL2R endodomain showed increased cell viability compared to the reference CD28z after the cell culture with HLA-A2 expression cells. These differences were not observed after polyclonal activation of the Tregs demonstrating that the effect is dependent on CAR signalling (FIG. 9).

### Example 8—Treg Suppression Potency Test: Evaluate the Immunoregulatory Function of Tregs by Analysing the Modulation of Co-Stimulatory Molecules on B Cells

B cell expression of CD80 and CD86 after co-culture with Tregs was analysed to evaluate the capacity of Tregs to reduce the expression of co-stimulatory molecules on antigen presenting cells.

Tregs expressing the CD28z, Construct 1 and Construct 2 CARs showed increased suppressive function compared to untransduce or dCAR expressing Tregs. CD80 and CD86 expression on B cells is only downregulated after culture with Tregs that signal through the CAR molecule (FIG. 10).

### Example 9—Tregs Transduced with a Construct Encoding FOXP3 and an HLA-A2 Specific CAR Express Both Genes and Express FOXP3 at Noticeably Higher Levels than Tregs with Endogenous FOXP3 Only

Tregs were isolated from PBMCs by CD4+ and CD25+ enrichment. The enriched cells were stained for CD4, CD25, CD127 and CD45RA and sorted by FACS.

Enriched Tregs were transduced with one of four constructs. FIG. 11A shows a schematic diagram of the constructs used: Construct F-C: illustrates a construct encoding 5′-FOXP3-P2A-A2 CAR-3′; Construct R-C: illustrates a construct encoding 5′-R-P2A-A2 CAR-3′, where R is another gene; Construct C: illustrates a construct encoding the A2 CAR only; Construct C-R: illustrates a construct encoding 5′-A2 CAR-P2A-R-3′, where R is another gene.

FIG. 11B shows a schematic of the transduction method.

The enriched and transduced cells were expanded using the following protocol:
Day 0, 0.25×10⁶ per ml in TexMACS™ with Gibco™ Dynabeads™ Human T-Activator CD3/CD28
Day 2, 0.1×10⁶ per ml in TexMACS™ on retronectin coated plates with IL-2 and virus
Day 4-9, resuspension to 0.25×10⁶ per ml in TexMACS™ with IL-2 in T25 flasks/6 well plates
Day 15, resuspension to 0.25×10⁶ per ml in TexMACS™ with IL-2 in T25 flasks

FIG. 12 shows the HLA-A2-specific CAR (A2 CAR) expression levels, FOXP3 expression levels and the expression levels of another gene, R, in Tregs transduced with Construct F-C, Construct R-C, Construct C, and Construct C-R, compared to mock control Tregs, as determined by flow cytometry.

FIG. 12A shows that Tregs transduced with each construct expressed the HLA-A2-specific CAR. The HLA-A2-specific CAR was expressed from both constructs in which the CAR was downstream (Construct R-C) and when the CAR was upstream (Construct C-R).

FIG. 12B shows that FOXP3 was expressed in all Tregs, but was noticeably higher in Construct F-C, particularly when compared to expression of the HLA-A2-specific CAR alone (Construct C).

### Example 9B—Tregs Transduced with a Construct Encoding FOXP3 and an HLA-A2 Specific CAR Retain FOXP3 Expression

FIG. 13 shows the HLA-A2-specific CAR (A2 CAR) expression levels, FOXP3 expression levels and the expression levels of another gene, R, in extended expanded Tregs transduced with Construct F-C, Construct R-C, Construct C, and Construct C-R, compared to mock control Tregs, as determined by flow cytometry.

FIG. 13A shows that Tregs transduced with each construct still expressed the HLA-A2-specific CAR after extended expansion.

FIG. 13B shows that FOXP3 expression decreased in Tregs transduced with Construct R-C, Construct C, and Construct C-R after extended expansion. In contrast, FOXP3 expression did not decrease in Tregs transduced Construct F-C after extended expansion. Consequently, FOXP3 expression was substantially higher in Construct F-C after extended expansion, particularly when compared to expression of the HLA-A2-specific CAR alone (Construct C).

### Example 10

Constructs as set out in FIG. 15 were manufactured and Treg cells were isolated, transduced and activated as described below. The control construct and constructs CI to CX have the sequences set out in SEQ ID NOS: 182 to 192 respectively. It will be noted that constructs CI-CX are also referred to interchangeably herein as constructs 1 to 10, or Con1 to Con10.

Constructs CI to CX and the control construct all comprise the same extracellular domain, including an extracellular hinge domain, and the same transmembrane domain. The extracellular domain comprises the hCD8 leader sequence of SEQ ID NO: 41, the anti HLA.A2 scFv sequence of SEQ ID NO: 196, and the CD8 hinge sequence of SEQ ID NO: 194. The transmembrane domain is the CD8 transmembrane domain of SEQ ID NO: 195. The constructs also all comprise a CD28 co-stimulatory domain (comprising a single amino acid addition to SEQ ID NO: 28 (penultimate A, which was introduced to facilitate cloning)) and a CD3 zeta signaling domain (as set out in SEQ ID NO: 27). The constructs differ with respect to the presence of a IL2Rβ truncated domain (IL2RBY510; SEQ ID NO: 23) and/or IL2Ry truncated domain (ILR2GT52; SEQ ID NO: 25) and the linker sequence between the ILR2B and ILR2G domains.

The sequence of construct CII is presented below (SEQ ID NO: 184):

MALPVTALLLPLALLLHAARPQVQLVQSGGGVVQPGGSLRVSCAASGV
TLSDYGMHWVRQAPGKGLEWVAFIRNDGSDKYYADSVKGRFTISRDNS
EKTVSLQMSSLRAEDTAVYYCAKNGESGPLDYWYLDLWGRGT [symbol]
DVVMTQSPSSLSASVGDRVTITCQSS
LDISHYLNWYQQKPGKAPKLLIYDASNLETGVPSRFSGSGSGTHFTFT
ISSLQPEDFATYYCQQYDNLPLTFGGGTKLEIKRTRKFVPVFLPAKPT
TTPAPRPPTPAPTIASQPLSLRPEACRPAAGGAVHTRGLDFACDIYIW
APLAGTCGVLLLSLVITLYCNHRPPAWVRSKRSRLLHSDYMNMTPRRP
GPTRKHYQPYAPPRDFAAYRaSERTMPRIPTLKNLEDLVTEYHGNFSA
WSGVSKGLAESLQPDYSERLCLVSEIleNCRNTGPWLKKVLKCNTPDP
SKFFSQLSSEHGGDVQKWLSSPFPSSSFSPGGLAPEISPLEVLERDKV
TQLLPLNTDAYLSLQELQGQDPTHLVctRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR

Starting from the N terminal the constituent parts of this construct may be identified as follows: the leader sequence in small letters; the scFv in plain letters with the scFv linker sequence between VH and VL in paler type face; intervening sequence underlined; the CD8 hinge domain in bold; the CD8 TM domain in italics; CD8 and CD28 intracellular domain parts in small letters; the CD28 co-stimulatory domain in italics (incorporating additional amino acid A shown in lower case at the penultimate position); ILR2GT52 underlined; added amino acids LE shown in lower case; ILRBY510 in bold; added amino acids CT shown in lower case; CD3 zeta in plain letters. Amino acids shown in lower case been added to allow for cloning.

Construct CII is a comparative construct comprising both truncated IL2Ry (IL2RGT52) and truncated IL2Rβ (IL2RBY510) domains, wherein the domains are joined directly to one another without a linker sequence in between.

Construct CI and CIII are also comparative constructs. CI differs from CII in the absence of ILR2GT52 and additional amino acids LE. Construct CIII differs from CII in the absence of ILR2RBY510 and additional amino acids CT.

Constructs IV to CX represent constructs of the invention and differ from CII in the presence of a linker sequence between added amino acids “le” at the end of IL2RGT52 and ILR2RBY510, corresponding respectively to the linker sequences of SEQ ID NOS: 155, 158, 159, 160, 166, 167 and 168 respectively, as depicted in FIG. 15.

The control construct differs from CII in the in the absence of ILR2GT52 and additional amino acids LE and in the absence of ILR2RBY510 and additional amino acids CT.

Although presented as examples herein, the design of the intracellular endodomain of these constructs is of more general applicability and may be used more broadly in the design and preparation of other chimeric receptors according to the invention. Thus, the endodomain of any one of constructs CIV to CX, or a variant thereof as described above, may be used in any chimeric receptor of the invention, including for example with any other extracellular domain or antigen-binding domain, and/or any other TM domain. Further, the design of the endodomain may be modified, for example to add one or more other costimulatory domains, or other intracellular features. Further, the endodomain sequence may be modified to remove all or part of the CD8 and CD28 intracellular domain parts (NHRPPAWV, SEQ ID NO: 205).

The endodomain of each of constructs CIV to CX is represented by SEQ ID NOS: 198 to 204 respectively.

Transduction and Expansion of cells

CD4⁺ CD25⁺ CD127⁻ human Tregs were isolated from healthy donor blood. Tregs were transduced using a lentiviral vector encoding constructs as shown in FIG. 15. Tregs were expanded using Dynabeads™ Human T-Activator CD3/CD28 for T Cell Expansion and Activation (ThermoFisher Scientific, Massachusetts, USA), and interleukin-2 (IL-2; Proleukin®, Clinigen, Burton upon Trent, UK). IL-2 was supplemented at 300 international units (IU) per 1 ml of culture volume.

### Resting of Tregs

Treg cultures were depleted of the Dynabeads™ Human T-Activator CD3/CD28 for T Cell Expansion and Activation (ThermoFisher Scientific) via magnetic technology. Cells were washed and resuspended in X-VIVO™ 15 media (Lonza); no IL-2 was supplemented.

### HLA-A2-Ig Artificial Presenting Cells (APCs)

1 mL (equating to approximately 400 million beads) of Dynabeads™ M-450 Epoxy beads were washed with sterile borate buffer (ThermoFisher Scientific). 40 μg of HLA-A2-Ig dimer (BD, New Jersey, USA) and 40 μg of anti-human CD28 mAB (BioXCell, New Hampshire, USA) were added to the Dynabeads™ M-450 Epoxy beads in borate buffer and incubated for 24 hours. After 24 hours, the borate buffer was removed and bead wash buffer and 0.1 g/L sodium azide (both ThermoFisher Scientific)) was added.

### Cell Expression Assays

2×10⁵ cells were washed in FACS before staining with APC-conjugated HLA-A*0201/CINGVCWTV dextramers (Immudex, Copenhagen, Denmark). Cells were washed in PBS and LIVE/DEAD™ Fixable Near-IR (ThermoFisher Scientific) was used to stain the dead cells. Cells were washed in FACS buffer before Fc blocking and staining with cell surface antibodies: Brilliant Violet™ anti-human CD4 (Biolegend, California, USA), PE/Cyanine7 anti-human CD25 (Biolegend) and CD34 monoclonal antibody (QBEND/10)-PE (ThermoFisher Scientific). Washed cells were then stained with Alexa Fluor® 488 anti-human FoxP3 antibodies. Cells were acquired on the Attune N×T Cytometer (ThermoFisher Scientific).

### Activation Assays

HLA-A1 and HLA-A2 K562 cells were irradiated before use. Activation assay co-cultures were set up as follows: Tregs:HLA-A1 K562s; Tregs:HLA-A2 K562s; Tregs:Dynabeads™ Human T-Activator CD3/CD28 for T Cell Expansion and Activation (ThermoFisher Scientific) or Tregs with media alone (unstimulated). Co-cultures were set up using X-VIVO™15 media (Lonza) supplemented with 5% heat-inactivated human serum (Merck Life Science UK Limited). Co-cultures were incubated at 37° C. and 5% CO₂ for 18 hours prior to staining. After 18 hours, cells were washed in PBS and LIVE/DEAD™ Fixable Near-IR (ThermoFisher Scientific) was used to stain the dead cells. Cells were washed in FACS buffer before Fc blocking and staining with cell surface antibodies: Brilliant Violet 510™ anti-human CD4 (Biolegend), CD34 monoclonal antibody (QBEND/10)-PE (ThermoFisher Scientific), PE-Cyanine7 anti-human CD69 (Biolegend) and APC anti-human CD137 (4-1BB; Biolegend). Washed cells were acquired on the Attune N×T Cytometer (ThermoFisher Scientific).

### Survival Assays

HLA-A1 and HLA-A2 K562 cells were irradiated before use. Survival assay co-cultures were set up as follows: Tregs:HLA-A1 K562s; Tregs:HLA-A2 K562s; Tregs:Dynabeads™ Human T-Activator CD3/CD28 for T Cell Expansion and Activation (ThermoFisher Scientific) or Tregs with media alone (unstimulated). Co-cultures were set up using 10 μg/mL Ultra-LEAF purified anti-human IL-2 and 5 μg/mL of purified anti-human CD122 (both Biolegend) in X-VIVO™ 15 media (Lonza) supplemented with 5% heat-inactivated human serum (Merck Life Science UK Limited). Co-cultures were incubated at 37° C. and 5% CO₂ for a total of 6 days. On day 6, co-cultures were washed in FACS buffer before Fc blocking and staining with cell surface antibodies: Brilliant Violet 510™ anti-human CD4 (Biolegend), PE/Cyanine7 anti-human CD25 (Biolegend) and CD34 monoclonal antibody (QBEND/10)-PE (ThermoFisher Scientific). Cells were then washed in FACS buffer and resuspended with Annexin V binding buffer with FITC Annexin V and 7AAD solutions using the FITC Annexin V Apoptosis Detection Kit with 7AAD (Biolegend). After the incubation, Annexin V binding buffer was added to the preparations. Cells were acquired on the Attune N×T Cytometer (ThermoFisher Scientific).

pSTAT5 ELISA and Western Blot (WB) Assays

pSTAT5 ELISA and WB assay co-cultures were set up as follows: Tregs:HLA-A2 K562s; Tregs: HLA-A2-Ig coated beads (A2 beads); Tregs with 100 IU/mL of IL-2 or Tregs with media alone (unstimulated). All co-cultures except for the addition of 100 IU/mL of IL-2 were set up using 10 μg/mL Ultra-LEAF purified anti-human IL-2 and 5 μg/mL of purified anti-human CD122 (both Biolegend). Co-cultures were set up in X-VIVO™15 media (Lonza) supplemented with 5% heat-inactivated human serum (Merck Life Science UK Limited) and incubated. Subsequently, the cells were washed twice with PBS and then cell pellets were lysed with radioimmunoprecipitation assay (RIPA) buffer (ThermoFisher Scientific). Samples were incubated on ice, prior to centrifugation at 14,000g. Cell lysate samples were stored at −80° C. until use.

STAT5 A/B (pY694/699) SimpleStep ELISA® Kit Assays

Cell lysate samples were defrosted and Pierce™ BCA Protein Assay Kit (ThermoFisher Scientific) was used to determine the protein concentration for optimal input amounts in the STAT5 A/B (pY694/699) SimpleStep ELISA® Kit assays (Abcam, Cambridge, UK). Samples were processed according to the manufacturer's guidelines.

pSTAT5 Western Blot Assays

Cell lysate samples were defrosted and Pierce™ BCA Protein Assay Kit (ThermoFisher Scientific) was used to determine the protein concentration for optimal input amounts in the pSTAT5 Western Blot assays. Equal amount of protein was loaded per well. Samples were treated at 70° C. for 10 mins before incubating on ice for 2 mins. Samples were then loaded into the NuPAGE™ 4-12% Bis-Tris Protein Gels which were placed in a XCell SureLock Mini-Cell gel running tank with 1× NuPAGE MES SDS Running Buffer (all ThermoFisher Scientific). Dry blotting using the iBlotTM 2 Dry Blotting System was performed according to the manufacturer's guidelines. Subsequently, the membrane was blocked and stained using Phospho-STAT5 (Tyr694) (Cell Signaling Technology, Massachusetts, USA).

### Results

Expression analysis in FIG. 16 shows that all constructs tested (see FIG. 15) were expressed on the cell surface of the Tregs (N from 2 to 16 individual donors) (FIG. 16A). Although the MFI of the constructs varied, all were capable of resulting in a high level of transduction across donors. Further, high and similar levels of transduction were seen in Treg samples for RQR8 (SEQ ID NO: 193) (safety switch co-expressed with CAR from nucleic acid construct). All cells regardless of CAR construct had similar levels of FOXP3.

All of the CAR constructs used were capable of being stimulated by antigen (HLA-A2 expressed on K562 A2 cells) as can be seen by FIGS. 17A and B which shows levels of two Treg activation markers (CD69 and CD137). The beads were also capable of stimulating the transduced Tregs non-specifically through their endogenous TCRs.

FIG. 18 shows % survival of the Tregs from three different donors when transduced with different CAR constructs. Constructs comprising a linker between the JAK3 binding motif and the STAT5 association motif/JAK1 binding motif generally resulted in an increased level of survival for cells transduced with those constructs. Particularly, constructs 4 (SEQ ID NO: 186) and 5 (SEQ ID NO: 187) (see FIG. 15) showed a higher percentage survival than construct 1 (SEQ ID NO: 183) indicating a potential advantage conferred by the presence of the JAK3 binding motif together with those linkers.

The data shown in FIG. 18 was re-normalised using CD69 rather than MFI, due to the non-linear relationship between MFI and CAR expression, see the results shown in FIG. 19. FIG. 19 further shows the survival increase seen when using constructs comprising a JAK3 binding domain together with a linker in addition to a STAT5 association motif/JAK1 binding motif, as compared to constructs with only a STAT5 association motif/JAK1 binding motif, or control constructs without either a JAK3 or a STAT5 association motif/JAK1 binding motif.

FIG. 20 further shows pSTAT5 activity associated with constructs comprising a JAK3 binding motif and a linker, according to ELISA and FIG. 21 shows pSTAT5 activity at increased levels when comparing construct 4 (SEQ ID NO: 186) (having a JAK3 binding motif and a linker) with construct 1 (SEQ ID NO: 183) which only has a STAT5 association motif/JAK1 binding motif pSTAT5 activity is indicative of the presence of persistence signal that the cells are receiving through the transduced constructs.

Thus, the data shown in Example 10 shows the survival advantage and increased pSTAT5 activity trend that can be seen across Treg donors when transduced with constructs additionally comprising a JAK3 binding motif and a linker.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A nucleic acid molecule comprising a polynucleotide sequence encoding a chimeric receptor, wherein said chimeric receptor comprises an endodomain comprising (i) a STAT5 association motif, and a JAK1 and/or JAK2 binding motif and (ii) a JAK3 binding motif, wherein (i) and (ii) are connected by a linker or hinge.

2. The nucleic acid of claim 1 wherein the chimeric receptor is a chimeric antigen receptor (CAR).

3. The nucleic acid of claim 1 or 2 wherein (i) the endodomain comprises two or more STAT5 association motifs, and/or the one or more STAT5 association motifs is from an interleukin receptor (IL) receptor endodomain.

4. The nucleic acid of any one of claims 1 to 3 wherein the one or more STAT5 association motifs is from IL2Rβ, IL7Rα, (CSF2Rβ), IL-9R, IL-17Rβ, erythropoietin receptor, thrombopoietin receptor, growth hormone receptor and prolactin receptor.

5. The nucleic acid molecule of any one of claims 1 to 4 wherein the STAT5 association motif comprises the amino acid motif YXXF/L (SEQ ID NO: 8); wherein X is any amino acid.

6. The nucleic acid molecule of any one of claims 1 to 5 wherein the STAT5 association motif comprises one or more of the amino acid motifs YCTF (SEQ ID NO: 9), YFFF (SEQ ID NO: 10), YLSL (SEQ ID NO: 11), and/or YLSLQ (SEQ ID NO: 12).

7. The nucleic acid molecule of claim 6 wherein the STAT5 association motif comprises the amino acid motif YLSLQ (SEQ ID NO: 12).

8. The nucleic acid molecule of claim 7 wherein the endodomain comprises a first STAT5 association motif comprising the amino acid motif YLSLQ (SEQ ID NO: 12) and a second STAT5 association motif comprising the amino acid motif YCTF (SEQ ID NO: 9) or YFFF (SEQ ID NO: 10).

9. The nucleic acid molecule of claim 8 wherein the endodomain comprises the following STAT5 association motifs: YLSLQ (SEQ ID NO: 12), YCTF (SEQ ID NO: 9) and YFFF (SEQ ID NO: 10).

10. The nucleic acid molecule of any one of claims 1 to 9 wherein the JAK binding motif is a JAK1 binding motif.

11. The nucleic acid molecule of claim 10 wherein the JAK1 binding motif is from an interleukin receptor (IL) receptor endodomain.

12. The nucleic acid molecule of any one of claims 1 to 11 wherein the JAK1 binding motif comprises an amino acid motif shown as any one of SEQ ID NO: 13-19 or a variant which has at least 80% identity to SEQ ID NO: 13-19.

13. The nucleic acid molecule of claim 12 wherein the JAK1 binding motif is the amino acid motif shown as SEQ ID NO: 13; or a variant which has at least 80% identity to SEQ ID NO: 13.

14. The nucleic acid molecule of any one of claims 1 to 13 wherein the chimeric receptor endodomain comprises an IL2Rβ endodomain shown as SEQ ID NO: 1; or a variant which has at least 80% sequence identity to SEQ ID NO: 1.

15. The nucleic acid molecule of any one of claims 1 to 14 wherein the chimeric receptor endodomain comprises a truncated IL2Rβ endodomain shown as any one of SEQ ID NO: 23 or 24; or a variant of SEQ ID NO: 23 or 24 which has at least 80% sequence identity thereto.

16. The nucleic acid molecule of any one of claims 1 to 15 wherein the JAK3 binding motif comprises SEQ ID NO: 25 or 26 or a variant which has at least 80% sequence identity to SEQ ID NO: 25 or 26.

17. A nucleic acid molecule of any one of claims 1 to 16 wherein the chimeric receptor endodomain comprises SEQ ID NO: 45, 53 or 154; or a variant which has at least 80% sequence identity to SEQ ID NO: 45, 53 or 154.

18. A nucleic acid molecule of any one of claims 1 to 17 wherein the JAK3 binding motif is positioned N terminal to the STAT5 association motif and JAK1 and/or JAK2 binding motifs in the chimeric receptor endodomain.

19. A nucleic acid molecule of any one of claims 1 to 18, wherein the linker or hinge comprises a sequence of any one of SEQ ID NOs 155-160.

20. A nucleic acid molecule of any one of claims 1 to 19, wherein the linker or hinge is a flexible linker or hinge.

21. A nucleic acid molecule of any one of claims 1 to 20, wherein the endodomain comprises any one of:
(i) SEQ ID NO: 25, SEQ ID NO: 155 and SEQ ID NO: 23,
(ii) SEQ ID NO: 25, SEQ ID NO: 158 and SEQ ID NO: 23, or
(iii) SEQ ID NO: 25, SEQ ID NO: 159 and SEQ ID NO: 23,
or a variant thereof having at least 80% sequence identity to (i), (ii) or (iii).

22. A nucleic acid molecule of any one of claims 1 to 21, where said encoded chimeric receptor does not comprise a first and a second heterodimerisation domain.

23. A vector comprising the nucleic acid molecule of any one of claims 1 to 22.

24. A chimeric receptor, particularly a CAR, comprising an endodomain comprising (i) a STAT5 association motif, and a JAK1 and/or JAK2 binding motif and (ii) a JAK3 binding motif, wherein (i) and (ii) are connected by a linker or hinge.

25. A chimeric receptor of claim 24 wherein said chimeric receptor does not comprise a first and a second heterodimerisation domain.

26. A chimeric receptor encoded by a nucleic acid of any one of claims 1 to 22.

27. An engineered cell, particularly a T cell, or NK cell comprising the nucleic acid molecule of any one of claims 1 to 22, the vector of claim 23 or the chimeric receptor of any one of claims 24 to 26.

28. The engineered cell of claim 27 wherein said cell is a Treg.

29. A pharmaceutical composition comprising the engineered cell (particularly T cell or NK cell) of claim 27 or 28.

30. An engineered cell of claim 27 or 28 or a pharmaceutical composition of claim 29 for use in therapy.

31. An engineered Treg cell of claim 28 for use in induction of tolerance to a transplant;
treating and/or preventing graft-versus-host disease (GvHD), an autoimmune or allergic disease; to promote tissue repair and/or tissue regeneration; or to ameliorate inflammation (particularly chronic inflammation secondary to metabolic disorders).

32. A pharmaceutical composition of claim 29 for use in induction of tolerance to a transplant; treating and/or preventing graft-versus-host disease (GvHD), an autoimmune or allergic disease; to promote tissue repair and/or tissue regeneration; or to ameliorate inflammation (particularly chronic inflammation secondary to metabolic disorders).

33. A method of inducing tolerance to a transplant; treating and/or preventing graft-versus-host disease (GvHD), an autoimmune or allergic disease; or to promote tissue repair and/or tissue regeneration; or to ameliorate inflammation (particularly chronic inflammation secondary to metabolic disorders) which comprises the step of administering an engineered Treg cell as defined in claim 28 or a pharmaceutical composition comprising an engineered Treg as defined in claim 29 to a subject.

34. A method according to claim 33 which comprises the following steps:
(i) isolation or provision of a Treg-enriched cell sample from a subject;
(ii) transduction or transfection of the Treg cells with: a nucleic acid molecule of any one of claims 1 to 22; or a vector of claim 23; and
(iii) administering the Treg cells from (ii) to the subject.

35. Use of an engineered Treg as defined in claim 28 in the manufacture of a medicament for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation (particularly chronic inflammation secondary to metabolic disorders).

36. An engineered Treg or pharmaceutical composition for use according to any of claims 30 to 32; a method according to claim 33 or 34; or the use according to claim 35 wherein the subject is a transplant recipient undergoing immunosuppression therapy.

37. An engineered Treg or pharmaceutical composition for use; a method according to; or
the use according to claim 36 wherein the transplant is selected from a liver, kidney, heart, lung, pancreas, intestine, stomach, bone marrow, vascularized composite tissue graft, and skin transplant.

38. An engineered Treg or pharmaceutical composition for use; a method; or the use according to claim 37 wherein the transplant is a liver transplant.

39. An engineered Treg or pharmaceutical composition for use; a method or the use according to claim 38 wherein the CAR comprises an antigen binding domain which is capable of specifically binding to an antigen selected from: a HLA antigen present in the transplanted liver but not in the recipient, a liver-specific antigen such as NTCP, or an antigen whose expression is up-regulated during rejection or tissue inflammation such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

40. An engineered Treg or pharmaceutical composition for use; a method or the use according to claim 39 wherein the CAR comprises an antigen binding domain which is capable of specifically binding to a HLA antigen that is present in the graft donor but not in the graft recipient.

41. An engineered Treg or pharmaceutical composition for use; a method or the use according to claim 40 wherein the antigen is HLA-A2.

42. An engineered Treg or pharmaceutical composition for use; a method or the use according to claim 41 wherein the CAR comprises an antigen binding domain comprises SEQ ID NO: 34 or a variant of SEQ ID NO: 34 with at least 80% identity thereto.

43. An engineered Treg or pharmaceutical composition for use; a method or the use according to and of claims 30 to 42 wherein the autoimmune or allergic disease is selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); CIPD, multiple sclerosis, neurodegenerative diseases (e.g. ALS) and juvenile onset diabetes.

44. A method of producing an engineered Treg according to claim 28, comprising the following steps:
(i) isolation of a cell-containing sample from a subject or provision of a cell-containing sample; and
(ii) transduction or transfection of the cell-containing sample with one or more nucleic acid molecules of any one of claims 1 to 22 or with one or more vectors of claim 23, to provide a population of engineered cells;
wherein the cell-containing sample comprises Tregs and/or Tregs are enriched and/or generated from the cell-containing sample prior to or after step (ii).


SEQUENCE LISTING (205 sequences)

SEQ ID NO: 1 - protein, 286 aa, Artificial Sequence
  amino acids 266 to 551 of interleukin-2 receptor beta  (IL2RB), NP_000869.1 (IL2Rbeta endodomain)
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 195 aa, Artificial Sequence
  amino acids 265 to 459 of interleukin-7 receptor-alpha (IL7R-alpha), NP_002176.2
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 3 - protein, 230 aa, Artificial Sequence
  amino acids 292 to 521 of interleukin-9 receptor (IL9R), NP_002177.2
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 4 - protein, 569 aa, Artificial Sequence
  amino acids 257 to 825 of interleukin-4 receptor-alpha (IL4R-alpha), NPJD00409.1
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 5 - protein, 437 aa, Artificial Sequence
  amino acids 461 to 897 of IL3RB, NP_000386.1
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 6 - protein, 189 aa, Artificial Sequence
  amino acids 314 to 502 of IL17RB, NP_061195.2
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 7 - protein, 91 aa, Artificial Sequence
  IL7R-alpha 2Y truncated
KKRIKPIVWPSLPDHKKTLEHLCKKPRKNLNVSFNPESFLDCQIHRVDDIQARDEVEGFLQDTFPQQPILTSLGSNQEEAYVTMSSFYQNQ

SEQ ID NO: 8 - protein, 4 aa, Artificial Sequence
  STAT5 association motif
  misc_feature (2)..(3): Xaa can be any naturally occurring amino acid
  MISC_FEATURE (4)..(4): Xaa may be Phe or Leu
YXXX

SEQ ID NO: 9 - protein, 4 aa, Artificial Sequence
  STAT5 association motif
YCTF

SEQ ID NO: 10 - protein, 4 aa, Artificial Sequence
  STAT5 association motif
YFFF

SEQ ID NO: 11 - protein, 4 aa, Artificial Sequence
  STAT5 association motif
YLSL

SEQ ID NO: 12 - protein, 5 aa, Artificial Sequence
  STAT5 association motif
YLSLQ

SEQ ID NO: 13 - protein, 57 aa, Artificial Sequence
  JAK1-binding motif
KVLKCNTPDPSKFFSQLSSEHGGDVQKWLSSPFPSSSFSPGGLAPEISPLEVLERDK

SEQ ID NO: 14 - protein, 45 aa, Artificial Sequence
  JAK1-binding motif
NPWFQRAKMPRALDFSGHTHPVATFQPSRPESVNDLFLCPQKELT

SEQ ID NO: 15 - protein, 42 aa, Artificial Sequence
  JAK1-binding motif
GYICLRNSLPKVLNFHNFLAWPFPNLPPLEAMDMVEVIYINR

SEQ ID NO: 16 - protein, 42 aa, Artificial Sequence
  JAK1-binding motif
PLKEKSIILPKSLISVVRSATLETKPESKYVSLITSYQPFSL

SEQ ID NO: 17 - protein, 43 aa, Artificial Sequence
  JAK1-binding motif
RRRKKLPSVLLFKKPSPFIFISQRPSPETQDTIHPLDEEAFLK

SEQ ID NO: 18 - protein, 45 aa, Artificial Sequence
  JAK1-binding motif
YIHVGKEKHPANLILIYGNEFDKRFFVPAEKIVINFITLNISDDS

SEQ ID NO: 19 - protein, 41 aa, Artificial Sequence
  JAK1-binding motif
RYVTKPPAPPNSLNVQRVLTFQPLRFIQEHVLIPVFDLSGP

SEQ ID NO: 20 - protein, 43 aa, Artificial Sequence
  JAK2-binding motif
NYVFFPSLKPSSSIDEYFSEQPLKNLLLSTSEEQIEKCFIIEN

SEQ ID NO: 21 - protein, 49 aa, Artificial Sequence
  JAK2-binding motif
YWFHTPPSIPLQIEEYLKDPTQPILEALDKDSSPKDDVWDSVSIISFPE

SEQ ID NO: 22 - protein, 51 aa, Artificial Sequence
  JAK2-binding motif
YAFSPRNSLPQHLKEFLGHPHHNTLLFFSFPLSDENDVFDKLSVIAEDSES

SEQ ID NO: 23 - protein, 94 aa, Artificial Sequence
  truncated IL2Rbeta endodomain - Y510
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 24 - protein, 208 aa, Artificial Sequence
  truncated IL2Rbeta endodomain - Y510 & Y392
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 25 - protein, 52 aa, Artificial Sequence
  JAK3-binding motif
ERTMPRIPTLKNLEDLVTEYHGNFSAWSGVSKGLAESLQPDYSERLCLVSEI

SEQ ID NO: 26 - protein, 86 aa, Artificial Sequence
  JAK3-binding motif
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 27 - protein, 113 aa, Artificial Sequence
  intracellular signaling domain of human CD3 zeta chain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 41 aa, Artificial Sequence
  CD28 signaling domain
RSKRSRLLHSDYMNMTPRRPGPTRKHYQPYAPPRDFAAYRS

SEQ ID NO: 29 - protein, 48 aa, Artificial Sequence
  CD27 signaling domain
QRRKYRSNKGESPVEPAEPCHYSCPREEEGSTIPIQEDYRKPEPACSP

SEQ ID NO: 30 - protein, 42 aa, Artificial Sequence
  OX40 signalling domain
ALYLLRRDQRLPPDAHKPPGGGSFRTPIQEEQADAHSTLAKI

SEQ ID NO: 31 - protein, 42 aa, Artificial Sequence
  41BB signalling domain
KRGRKKLLYIFKQPFMRPVQTTQEEDGCSCRFPEEEEGGCEL

SEQ ID NO: 32 - protein, 38 aa, Artificial Sequence
  ICOS signalling domain
CWLTKKKYSSSVHDPNGEYMFMRAVNTAKKSRLTDVTL

SEQ ID NO: 33 - protein, 197 aa, Artificial Sequence
  TNFRSF25 signalling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 34 - protein, 246 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLG

SEQ ID NO: 35 - protein, 27 aa, Artificial Sequence
  transmembrane domain, CD28 transmembrane
FWVLVVVGGVLACYSLLVTVAFIIFWV

SEQ ID NO: 36 - protein, 66 aa, Artificial Sequence
  transmembrane domain, CD28 hinge and transmembrane sequence
IEVMYPPPYLDNEKSNGTIIHVKGKHLCPSPLFPGPSKPFWVLVVVGGVLACYSLLVTVAFIIFWV

SEQ ID NO: 37 - protein, 68 aa, Artificial Sequence
  transmembrane and intracellular portion of human CD28
FWVLVVVGGVLACYSLLVTVAFIIFWVRSKRSRLLHSDYMNMTPRRPGPTRKHYQPYAPPRDFAAYRS

SEQ ID NO: 38 - protein, 27 aa, Artificial Sequence
  transmembrane domain, CD28
FWVLVVVGGVLACYSLLVTVAFIIFWV

SEQ ID NO: 39 - protein, 10 aa, Artificial Sequence
  c-Myc tag
EQKLISEEDL

SEQ ID NO: 40 - protein, 70 aa, Artificial Sequence
  CD28 hinge/transmembrane domain with integrated c-Myc tag
IEVEQKLISEEDLLDNEKSNGTIIHVKGKHLCPSPLFPGPSKPFWVLVVVGGVLACYSLLVTVAFIIFWV

SEQ ID NO: 41 - protein, 21 aa, Artificial Sequence
  leader sequence
MALPVTALLLPLALLLHAARP

SEQ ID NO: 42 - protein, 834 aa, Artificial Sequence
  chimeric antigen receptor (CAR) containing HLA-A2 scFV, c-Myc tag, CD28, IL2RB-Y510, CD3 zeta endodomain
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRETRGGGATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 43 - protein, 639 aa, Artificial Sequence
  CAR containing HLA-A2 scFV, c-Myc tag, CD28, IL2RG-T52, IL2RB-Y510, CD3 zeta endodomain
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERTMPRIPTLKNLEDLVTEYHGNFSAWSGVSKGLAESLQPDYSERLCLVSEINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 657 aa, Artificial Sequence
  CAR containing HLA-A2 scFV, c-Myc tag, CD28, IL2RG-T52, IL7RA-2Y, CD3 zeta endodomain
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERTMPRIPTLKNLEDLVTEYHGNFSAWSGVSKGLAESLQPDYSERLCLVSEIKKRIKPIVWPSLPDHKKTLEHLCKKPRKNLNVSFNPESFLDCQIHRVDDIQARDEVEGFLQDTFPQQPILTSLGSNQEEAYVTMSSFYQNQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 45 - protein, 299 aa, Artificial Sequence
  illustrative endodomain sequence comprising CD28, IL2RG-T52, IL2RB-Y510, CD3 zeta signalling domains
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 22 aa, Artificial Sequence
  P2A peptide - cleavage domain
GSGATNFSLLKQAGDVEENPGP

SEQ ID NO: 47 - protein, 21 aa, Artificial Sequence
  T2A peptide - cleavage domain
GSGEGRGSLLTCGDVEENPGP

SEQ ID NO: 48 - protein, 23 aa, Artificial Sequence
  E2A peptide - cleavage domain
GSGQCTNYALLKLAGDVESNPGP

SEQ ID NO: 49 - protein, 25 aa, Artificial Sequence
  F2A peptide - cleavage domain
GSGVKQTLNFDLLKLAGDVESNPGP

SEQ ID NO: 50 - protein, 4 aa, Artificial Sequence
  Furin site - cleavage domain
  misc_feature (2)..(3): Xaa can be any naturally occurring amino acid
RXXR

SEQ ID NO: 51 - protein, 4 aa, Artificial Sequence
  Furin site - cleavage domain
RRKR

SEQ ID NO: 52 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 53 - protein, 317 aa, Artificial Sequence
  illustrative endodomain sequence comprising CD28, IL2RG-T52, IL7RA-2Y, CD3 zeta signalling domains
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEIKKRIKPIVWPSLPDHKKTLEHLCKKPRKNLNVSFNPESFLDCQIHRVDDIQARDEVEGFLQDTFPQQPILTSLGSNQEEAYVTMSSFYQNQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 54 - protein, 120 aa, Artificial Sequence
  variable heavy domain
VQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTV

SEQ ID NO: 55 - protein, 117 aa, Artificial Sequence
  variable heavy domain
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGT

SEQ ID NO: 56 - protein, 117 aa, Artificial Sequence
  variable heavy domain
QVQLVQSGGGVVQPGGSMRVSCAASGVTLSDYGMHWVRQAPGKGLEWVAFIRNDGSDKYYADSVRGRFTISRDNSKKTVFLQMNSLRAEDTAVYYCAKNGESGPLDYWYFDLWGRGT

SEQ ID NO: 57 - protein, 117 aa, Artificial Sequence
  variable heavy domain
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGT

SEQ ID NO: 58 - protein, 108 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLG

SEQ ID NO: 59 - protein, 107 aa, Artificial Sequence
  variable light domain
DVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 60 - protein, 107 aa, Artificial Sequence
  variable light domain
DIVLMQSPSFLSASVGDRVTITCRASHGINNYLAWYQQKPGKAPKLLIYAASTLQSGVPSRFSGSGSGTEFTLTISSLQPEDFATYYCQQYDSYPPTFGRTKVEIKR

SEQ ID NO: 61 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIK

SEQ ID NO: 62 - protein, 107 aa, Artificial Sequence
  variable light domain
DVVMTQSPSSLSASVGDRVTITCQASQDISNYLNWYQQEPGKAPKLLIYDETHLDSGVPSRFTGSRSGTDFTLTISSLQPEDFATYYCQQYDSLPPTFGGGTKVDIK

SEQ ID NO: 63 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPITFGGGTKVDIK

SEQ ID NO: 64 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPSTFGGGTKVDIK

SEQ ID NO: 65 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDFGTYYCQQYNTYPLTFGGGTKVDIK

SEQ ID NO: 66 - protein, 107 aa, Artificial Sequence
  variable light domain
DVVMTQSPSSLTASVGDRVTITCQASQDISNYLNWYQQKPGKAPKLLIYDASNLETGVPSRFSGSGSGTDFTLSIDSLQPEDFATYYCQQYHTYPLTFGGGTKVDIK

SEQ ID NO: 67 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVDIK

SEQ ID NO: 68 - protein, 107 aa, Artificial Sequence
  variable light domain
DVVMTQSPSSLSASVGDRVTITCRTSQGISSALAWYQQKPGKAPKLLIYDASSLESGVPSRFSGSGSGTDFTLTISSLQPEDFATYYCQQFNNYPLTFGGGTKVDIK

SEQ ID NO: 69 - protein, 107 aa, Artificial Sequence
  variable light domain
DVVMTQSPSSLSASVGDRVTITCQASQDISNYLAWYQQKPGRAPTLLIFAASNLQSGVPSRFSGSGSGTEFTLTISGLQPEDFATYYCLQDSSYPPTFGGGTKVDIK

SEQ ID NO: 70 - protein, 107 aa, Artificial Sequence
  variable light domain
DVVMTQSPSTLSASVGDRVTITCRASQSISSWLAWYQQKPGRAPTLLIYKASNLQSGVPSRFSGSGSGTEFTLTISSLQPDDFASYYCQQYSNYPLTFGGGTKVDIK

SEQ ID NO: 71 - protein, 107 aa, Artificial Sequence
  variable light domain
DVVMTQSPSFLSASVGDRVTITCRASHGISNYFAWYQQKPGKAPKLLIYATSTLQSGVPSRFSGSGSGTEFTLTISGLQPEDFATYYCQQYSSYPLTFGGGTKVDIK

SEQ ID NO: 72 - protein, 106 aa, Artificial Sequence
  variable light domain
DVVMTQSPSTLSAYVGDRITITCRASRGSNYLAWYQQKPGKAPKLLIYATSTLQSGVPLRFSGSGSGTEFTLTISGLQPEDFATYYCQQYDSYPPTFGGGTKVDIK

SEQ ID NO: 73 - protein, 238 aa, Artificial Sequence
  antigen binding domain sequence which is capable of binding to HLA-A2
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGSSGGGGSGGGGSGGGGSDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 74 - protein, 241 aa, Artificial Sequence
  antigen binding domain sequence which is capable of binding to HLA-A2
QVQLVQSGGGVVQPGGSMRVSCAASGVTLSDYGMHWVRQAPGKGLEWVAFIRNDGSDKYYADSVRGRFTISRDNSKKTVFLQMNSLRAEDTAVYYCAKNGESGPLDYWYFDLWGRGTSSGGGGSGGGGSGGGGSDIVLMQSPSFLSASVGDRVTITCRASHGINNYLAWYQQKPGKAPKLLIYAASTLQSGVPSRFSGSGSGTEFTLTISSLQPEDFATYYCQQYDSYPPTFGRTKVEIKR

SEQ ID NO: 75 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIK

SEQ ID NO: 76 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQEPGKAPKLLIYDETHLDSGVPSRFTGSRSGTDFTLTISSLQPEDFATYYCQQYDSLPPTFGGGTKVDIK

SEQ ID NO: 77 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPITFGGGTKVDIK

SEQ ID NO: 78 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPSTFGGGTKVDIK

SEQ ID NO: 79 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFGTYYCQQYNTYPLTFGGGTKVDIK

SEQ ID NO: 80 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLTASVGDRVTITCQASQDISNYLNWYQQKPGKAPKLLIYDASNLETGVPSRFSGSGSGTDFTLSIDSLQPEDFATYYCQQYHTYPLTFGGGTKVDIK

SEQ ID NO: 81 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVDIK

SEQ ID NO: 82 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCRTSQGISSALAWYQQKPGKAPKLLIYDASSLESGVPSRFSGSGSGTDFTLTISSLQPEDFATYYCQQFNNYPLTFGGGTKVDIK

SEQ ID NO: 83 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLAWYQQKPGRAPTLLIFAASNLQSGVPSRFSGSGSGTEFTLTISGLQPEDFATYYCLQDSSYPPTFGGGTKVDIK

SEQ ID NO: 84 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSTLSASVGDRVTITCRASQSISSWLAWYQQKPGRAPTLLIYKASNLQSGVPSRFSGSGSGTEFTLTISSLQPDDFASYYCQQYSNYPLTFGGGTKVDIK

SEQ ID NO: 85 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSFLSASVGDRVTITCRASHGISNYFAWYQQKPGKAPKLLIYATSTLQSGVPSRFSGSGSGTEFTLTISGLQPEDFATYYCQQYSSYPLTFGGGTKVDIK

SEQ ID NO: 86 - protein, 240 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSTLSAYVGDRITITCRASRGSNYLAWYQQKPGKAPKLLIYATSTLQSGVPLRFSGSGSGTEFTLTISGLQPEDFATYYCQQYDSYPPTFGGGTKVDIK

SEQ ID NO: 87 - protein, 21 aa, Artificial Sequence
  transmembrane domain, CD8alpha transmembrane
IYIWAPLAGTCGVLLLSLVIT

SEQ ID NO: 88 - protein, 72 aa, Artificial Sequence
  CD8alpha hinge domain and CD28 transmembrane domain
TTTPAPRPPTPAPTIASQPLSLRPEACRPAAGGAVHTRGLDFACDFWVLVVVGGVLACYSLLVTVAFIIFWV

SEQ ID NO: 89 - protein, 60 aa, Artificial Sequence
  CD28 hinge domain and CD8alpha transmembrane domain
IEVMYPPPYLDNEKSNGTIIHVKGKHLCPSPLFPGPSKPIYIWAPLAGTCGVLLLSLVIT

SEQ ID NO: 90 - protein, 5 aa, Artificial Sequence
  GL heavy chain variable region (VH) complementarity determining region (CDR), GL VH CDR1
DYGMH

SEQ ID NO: 91 - protein, 17 aa, Artificial Sequence
  GL VH CDR2
FIRNDGSDKYYADSVKG

SEQ ID NO: 92 - protein, 14 aa, Artificial Sequence
  GL VH CDR3
NGESGPLDYWYFDL

SEQ ID NO: 93 - protein, 5 aa, Artificial Sequence
  3PB2 VH CDR1
DYGMH

SEQ ID NO: 94 - protein, 17 aa, Artificial Sequence
  3PB2 VH CDR2
FIRNDGSDKYYADSVKG

SEQ ID NO: 95 - protein, 14 aa, Artificial Sequence
  3PB2 VH CDR3
NGESGPLDYWYLDL

SEQ ID NO: 96 - protein, 5 aa, Artificial Sequence
  3PC4 VH CDR1
DYGMH

SEQ ID NO: 97 - protein, 17 aa, Artificial Sequence
  3PC4 VH CDR2
FIRNDGSDKYYADSVRG

SEQ ID NO: 98 - protein, 14 aa, Artificial Sequence
  3PC4 VH CDR3
NGESGPLDYWYFDL

SEQ ID NO: 99 - protein, 5 aa, Artificial Sequence
  3PF12 VH CDR1
DYGMH

SEQ ID NO: 100 - protein, 17 aa, Artificial Sequence
  3PF12 VH CDR2
FIRNDGSDKYYADSVKG

SEQ ID NO: 101 - protein, 14 aa, Artificial Sequence
  3PF12 VH CDR3
NGESGPLDYWYFDL

SEQ ID NO: 102 - protein, 11 aa, Artificial Sequence
  GL light chain variable region (VL) CDR1
QASQDISNYLN

SEQ ID NO: 103 - protein, 7 aa, Artificial Sequence
  GL VL CDR2
DASNLET

SEQ ID NO: 104 - protein, 9 aa, Artificial Sequence
  GL VL CDR3
QQYDNLPPT

SEQ ID NO: 105 - protein, 11 aa, Artificial Sequence
  3PB2 VL CDR1
QSSLDISHYLN

SEQ ID NO: 106 - protein, 7 aa, Artificial Sequence
  3PB2 VL CDR2
DASNLET

SEQ ID NO: 107 - protein, 9 aa, Artificial Sequence
  3PB2 VL CDR3
QQYDNLPLT

SEQ ID NO: 108 - protein, 11 aa, Artificial Sequence
  3PC4 VL CDR1
RASHGINNYLA

SEQ ID NO: 109 - protein, 7 aa, Artificial Sequence
  3PC4 VL CDR2
AASTLQS

SEQ ID NO: 110 - protein, 9 aa, Artificial Sequence
  3PC4 VL CDR3
QQYDSYPPT

SEQ ID NO: 111 - protein, 11 aa, Artificial Sequence
  3PF12 VL CDR1
QASQDISNYLN

SEQ ID NO: 112 - protein, 7 aa, Artificial Sequence
  3PF12 VL CDR2
DASNLET

SEQ ID NO: 113 - protein, 9 aa, Artificial Sequence
  3PF12 VL CDR3
QQYSSFPLT

SEQ ID NO: 114 - protein, 11 aa, Artificial Sequence
  C12 VL CDR1
QASQDISNYLN

SEQ ID NO: 115 - protein, 7 aa, Artificial Sequence
  C12 VL CDR2
DETHLDS

SEQ ID NO: 116 - protein, 9 aa, Artificial Sequence
  C12 VL CDR3
QQYDSLPPT

SEQ ID NO: 117 - protein, 11 aa, Artificial Sequence
  E7 VL CDR1
QASQDISNYLN

SEQ ID NO: 118 - protein, 7 aa, Artificial Sequence
  E7 VL CDR2
DASNLET

SEQ ID NO: 119 - protein, 9 aa, Artificial Sequence
  E7 VL CDR3
QQYDNLPIT

SEQ ID NO: 120 - protein, 11 aa, Artificial Sequence
  H10 VL CDR1
QASQDISNYLN

SEQ ID NO: 121 - protein, 7 aa, Artificial Sequence
  H10 VL CDR2
DASNLET

SEQ ID NO: 122 - protein, 9 aa, Artificial Sequence
  H10 VL CDR3
QQYDNLPST

SEQ ID NO: 123 - protein, 11 aa, Artificial Sequence
  B8 VL CDR1
QASQDISNYLN

SEQ ID NO: 124 - protein, 7 aa, Artificial Sequence
  B8 VL CDR2
DASNLET

SEQ ID NO: 125 - protein, 9 aa, Artificial Sequence
  B8 VL CDR3
QQYNTYPLT

SEQ ID NO: 126 - protein, 11 aa, Artificial Sequence
  D2 VL CDR1
QASQDISNYLN

SEQ ID NO: 127 - protein, 7 aa, Artificial Sequence
  D2 VL CDR2
DASNLET

SEQ ID NO: 128 - protein, 9 aa, Artificial Sequence
  D2 VL CDR3
QQYHTYPLT

SEQ ID NO: 129 - protein, 11 aa, Artificial Sequence
  B10 VL CDR1
QASQDISNYLN

SEQ ID NO: 130 - protein, 7 aa, Artificial Sequence
  B10 VL CDR2
DASNLET

SEQ ID NO: 131 - protein, 9 aa, Artificial Sequence
  B10 VL CDR3
QQYDNLPLT

SEQ ID NO: 132 - protein, 11 aa, Artificial Sequence
  2A9 VL CDR1
RTSQGISSALA

SEQ ID NO: 133 - protein, 7 aa, Artificial Sequence
  2A9 VL CDR2
DASSLES

SEQ ID NO: 134 - protein, 9 aa, Artificial Sequence
  2A9 VL CDR3
QQFNNYPLT

SEQ ID NO: 135 - protein, 11 aa, Artificial Sequence
  3B12 VL CDR1
QASQDISNYLA

SEQ ID NO: 136 - protein, 7 aa, Artificial Sequence
  3B12 VL CDR2
AASNLQS

SEQ ID NO: 137 - protein, 9 aa, Artificial Sequence
  3B12 VL CDR3
LQDSSYPPT

SEQ ID NO: 138 - protein, 11 aa, Artificial Sequence
  2D4 VL CDR1
RASQSISSWLA

SEQ ID NO: 139 - protein, 7 aa, Artificial Sequence
  2D4 VL CDR2
KASNLQS

SEQ ID NO: 140 - protein, 9 aa, Artificial Sequence
  2D4 VL CDR3
QQYSNYPLT

SEQ ID NO: 141 - protein, 11 aa, Artificial Sequence
  3D4 VL CDR1
RASHGISNYFA

SEQ ID NO: 142 - protein, 7 aa, Artificial Sequence
  3D4 VL CDR2
ATSTLQS

SEQ ID NO: 143 - protein, 9 aa, Artificial Sequence
  3D4 VL CDR3
QQYSSYPLT

SEQ ID NO: 144 - protein, 10 aa, Artificial Sequence
  B3 VL CDR1
RASRGSNYLA

SEQ ID NO: 145 - protein, 7 aa, Artificial Sequence
  B3 VL CDR2
ATSTLQS

SEQ ID NO: 146 - protein, 9 aa, Artificial Sequence
  B3 VL CDR3
QQYDSYPPT

SEQ ID NO: 147 - protein, 431 aa, Artificial Sequence
  Mutant of FOXP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPSRCSNPTPGP

SEQ ID NO: 148 - protein, 431 aa, Artificial Sequence
  Mutant of FOXP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPARCSNPTPGP

SEQ ID NO: 149 - protein, 431 aa, Artificial Sequence
  Mutant of FOXP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPARCSNPTPGP

SEQ ID NO: 150 - protein, 362 aa, Artificial Sequence
  N and C terminal FOXP3 truncation
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 151 - protein, 441 aa, Artificial Sequence
  Variant of FOXP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 152 - DNA, 1296 bp, Homo sapiens
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 153 - DNA, 1352 bp, Artificial Sequence
  Polynucleotide sequence encoding FOXP3 variant
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 154 - protein, 314 aa, Artificial Sequence
  CAR endodomain sequence
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEIGGGGSGGGGSGGGGSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 15 aa, Artificial Sequence
  Linker sequence
GGGGSGGGGSGGGGS

SEQ ID NO: 156 - protein, 5 aa, Artificial Sequence
  Linker sequence
GGGGS

SEQ ID NO: 157 - protein, 10 aa, Artificial Sequence
  Linker sequence
GGGGSGGGGS

SEQ ID NO: 158 - protein, 20 aa, Artificial Sequence
  Linker sequence
GGGGSGGGGSGGGGSGGGGS

SEQ ID NO: 159 - protein, 6 aa, Artificial Sequence
  Linker sequence
GGGGGG

SEQ ID NO: 160 - protein, 8 aa, Artificial Sequence
  Linker sequence
GGGGGGGG

SEQ ID NO: 161 - protein, 10 aa, Artificial Sequence
  Linker sequence
ETSGGGGSRL

SEQ ID NO: 162 - protein, 16 aa, Artificial Sequence
  Linker sequence
SGGGGSGGGGSGGGGS

SEQ ID NO: 163 - protein, 4 aa, Artificial Sequence
  Linker sequence
GGGS

SEQ ID NO: 164 - protein, 6 aa, Artificial Sequence
  Linker Sequence
GGGGGS

SEQ ID NO: 165 - protein, 7 aa, Artificial Sequence
  Linker sequence
GGGGGGS

SEQ ID NO: 166 - protein, 18 aa, Artificial Sequence
  Linker sequence
KESGSVSSEQLAQFRSLD

SEQ ID NO: 167 - protein, 14 aa, Artificial Sequence
  Linker sequence
EGKSSGSGSESKST

SEQ ID NO: 168 - protein, 12 aa, Artificial Sequence
  Linker sequence
GSAGSAAGSGEF

SEQ ID NO: 169 - protein, 16 aa, Artificial Sequence
  Linker sequence
SGGGGSAGSAAGSGEF

SEQ ID NO: 170 - protein, 16 aa, Artificial Sequence
  Linker sequence
SGGGLLLLLLLLGGGS

SEQ ID NO: 171 - protein, 16 aa, Artificial Sequence
  Linker sequence
SGGGAAAAAAAAGGGS

SEQ ID NO: 172 - protein, 24 aa, Artificial Sequence
  Linker sequence
SGGGAAAAAAAAAAAAAAAAGGGS

SEQ ID NO: 173 - protein, 24 aa, Artificial Sequence
  Linker sequence
SGALGGLALAGLLLAGLGLGAAGS

SEQ ID NO: 174 - protein, 14 aa, Artificial Sequence
  Linker sequence
SLSLSPGGGGGPAR

SEQ ID NO: 175 - protein, 25 aa, Artificial Sequence
  Linker sequence
SLSLSPGGGGGPARSLSLSPGGGGG

SEQ ID NO: 176 - protein, 6 aa, Artificial Sequence
  Linker sequence
GSSGSS

SEQ ID NO: 177 - protein, 7 aa, Artificial Sequence
  Linker sequence
GSSSSSS

SEQ ID NO: 178 - protein, 6 aa, Artificial Sequence
  Linker sequence
GGSSSS

SEQ ID NO: 179 - protein, 6 aa, Artificial Sequence
  Linker sequence
GSSSSS

SEQ ID NO: 180 - protein, 6 aa, Artificial Sequence
  Linker sequence
SGGGGS

SEQ ID NO: 181 - protein, 45 aa, Artificial Sequence
  CD8a hinge
TTTPAPRPPTPAPTIASQPLSLRPEACRPAAGGAVHTRGLDFACD

SEQ ID NO: 182 - protein, 512 aa, Artificial Sequence
  Control Construct
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 608 aa, Artificial Sequence
  QTX01-CI (Construct 1)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 662 aa, Artificial Sequence
  QTX01-CII (Construct 2)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILENCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 566 aa, Artificial Sequence
  QTX01-CIII (Construct 3)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILERVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 677 aa, Artificial Sequence
  QTX01-CIV (Construct 4)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGSGGGGSGGGGSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 682 aa, Artificial Sequence
  QTX01-CV (Construct 5)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGSGGGGSGGGGSGGGGSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 668 aa, Artificial Sequence
  QTX01-CVI (Construct 6)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGGGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 670 aa, Artificial Sequence
  QTX01-CVII (Construct 7)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGGGGGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 680 aa, Artificial Sequence
  QTX01-CVIII (Construct 8)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEKESGSVSSEQLAQFRSLDNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 676 aa, Artificial Sequence
  QTX01-CIX (Construct 9)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEEGKSSGSGSESKSTNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 192 - protein, 674 aa, Artificial Sequence
  QTX01-CX (Construct 10)
MALPVTALLLPLALLLHAARPQVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIKRTRKFVPVFLPAKPTTTPAPRPPTPAPTIASQPLSLRPEACRPAAGGAVHTRGLDFACDIYIWAPLAGTCGVLLLSLVITLYCNHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGSAGSAAGSGEFNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - protein, 157 aa, Artificial Sequence
  RQR8
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 194 - protein, 55 aa, Artificial Sequence
  CD8 hinge domain in Figure 15 constructs
FVPVFLPAKPTTTPAPRPPTPAPTIASQPLSLRPEACRPAAGGAVHTRGLDFACD

SEQ ID NO: 195 - protein, 24 aa, Artificial Sequence
  CD8 TM domain in Figure 15 constructs
IYIWAPLAGTCGVLLLSLVITLYC

SEQ ID NO: 196 - protein, 246 aa, Artificial Sequence
  ScFc used in Constructs 4-10 in Example 10
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 197 - protein, 5 aa, Artificial Sequence
  Furin cleavage site
IDGSG

SEQ ID NO: 198 - protein, 327 aa, Artificial Sequence
  CIV (Construct 4) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGSGGGGSGGGGSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 332 aa, Artificial Sequence
  CV (Construct 5) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGSGGGGSGGGGSGGGGSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 200 - protein, 318 aa, Artificial Sequence
  CVI (Construct 6) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGGGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 320 aa, Artificial Sequence
  CVII (Construct 7) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGGGGGGGGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 330 aa, Artificial Sequence
  CVIII (Construct 8) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEKESGSVSSEQLAQFRSLDNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 203 - protein, 323 aa, Artificial Sequence
  CIX (Construct 9) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEEGKSSGSGSESKSTNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQAL

SEQ ID NO: 204 - protein, 324 aa, Artificial Sequence
  CX (Construct 10) endodomain
NHRPPAWVRSKRSRLLHSDYMNMTPRRPGPTRKHYQPYAPPRDFAAYRASERTMPRIPTLKNLEDLVTEYHGNFSAWSGVSKGLAESLQPDYSERLCLVSEILEGSAGSAAGSGEFNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 205 - protein, 8 aa, Artificial Sequence
  CD8 and CD28 intracellular domain parts in Figure 15 constructs
NHRPPAWV